SEQUENCE LISTING (58 sequences)

SEQ ID NO: 1 - DNA, 71 bp, synthetic construct
  source 1..71: mol_type = other DNA
  misc_difference 17..50: note = Any nucleotide
cttcatgctggtctcannnnnnnnnnnnnnnnnnnnnnnnnnnnnnnnnncagtgctccttggctgatccg

SEQ ID NO: 2 - DNA, 87 bp, synthetic construct
  source 1..87: mol_type = other DNA
  modified_base 1: mod_base = OTHER; note = Phosphorylated nucleotide
  misc_difference 26..60: note = Any nucleotide
attccggatcagtcaaggagcactgnnnnnnnnnnnnnnnnnnnnnnnnnnnnnnnnnnnttccacttcctaatctgtcatcttctg

SEQ ID NO: 3 - skipped

SEQ ID NO: 4 - DNA, 300 bp, synthetic construct
  source 1..300: mol_type = other DNA
tggtctctgaatgccgccgttatccaggttgaatgaagtatcctggactgcagatgaataaactatgccaggctttgtggtggtgcacacgtccttgcgattaagtggacaccaggctgcagtgccaaaacgtaacctggttcgcgagtatatgtttacattccaggaggataagattcagcggcatccaggacctcactgttgcgcagtgaagaggtcctggatacaagaaacgaggttgtgccgattatacgtagtcgtatcctggtacgtcctggcgtctggaaggcttagagacca

SEQ ID NO: 5 - DNA, 36 bp, synthetic construct
  source 1..36: mol_type = other DNA
  misc_difference 1..15: note = Any nucleotide
nnnnnnnnnnnnnnncagtgctccttggctgatccg

SEQ ID NO: 6 - DNA, 40 bp, synthetic construct
  source 1..40: mol_type = other DNA
  modified_base 1: mod_base = OTHER; note = Phosphorylated nucleotide
  misc_difference 26..40: note = Any nucleotide
attccggatcagccaaggagcactgnnnnnnnnnnnnnnn

SEQ ID NO: 7 - DNA, 26 bp, synthetic construct
  source 1..26: mol_type = other DNA
  modified_base 1: mod_base = OTHER; note = Phosphorylated nucleotide
gcttgcactgagatttttctcagtgc

SEQ ID NO: 8 - DNA, 57 bp, synthetic construct
  source 1..57: mol_type = other DNA
gtgtcttttggtctttctggtgctcttcgaatcagaagatgacagattaggaagtgg

SEQ ID NO: 9 - DNA, 42 bp, synthetic construct
  source 1..42: mol_type = other DNA
  modified_base 1: mod_base = OTHER; note = Phosphorylated nucleotide
attcgaagagcaccagaaagaccaaaagacacagtcacagat

SEQ ID NO: 10 - RNA, 106 nt, synthetic construct
  source 1..106: mol_type = other RNA
tcggcgatctacgcagcgacatatatggcgacatttgtaattcctggaccgatacttccgtcaggacagaggttgccataacaacactctcctcatctgtctctcc

SEQ ID NO: 11 - DNA, 72 bp, synthetic construct
  source 1..72: mol_type = other DNA
cttcatgctggtctcagcttgatctcgaagagctacagcgccgtacagactcagtgctccttggctgatccg

SEQ ID NO: 12 - DNA, 40 bp, synthetic construct
  source 1..40: mol_type = other DNA
  modified_base 1: mod_base = OTHER; note = Phosphorylated nucleotide
attccggatcagccaaggagcactgagtctgtacggcgct

SEQ ID NO: 13 - DNA, 36 bp, synthetic construct
  source 1..36: mol_type = other DNA
gtctgtgaatggatgcagtgctccttggctgatccg

SEQ ID NO: 14 - DNA, 87 bp, synthetic construct
  source 1..87: mol_type = other DNA
attccggatcagtcaaggagcactgcatccattcacagacgtcgctgcgtagatcgccgattccacttcctaatctgtcatcttctg

SEQ ID NO: 15 - DNA, 58 bp, synthetic construct
  source 1..58: mol_type = other DNA
  modified_base 58: mod_base = OTHER; note = Phosphorylated nucleotide
tcggcgatctacgcagcgacgactgtacatgtcctcgcactggctcgctggatgtgtc

SEQ ID NO: 16 - DNA, 38 bp, synthetic construct
  source 1..38: mol_type = other DNA
gacacatccagcgagccagtgcgaggacatgtacagtc

SEQ ID NO: 17 - DNA, 53 bp, synthetic construct
  source 1..53: mol_type = other DNA
gccatctgtggacccatccaggcttgtgagactgtagctcttcgagatcaagc

SEQ ID NO: 18 - DNA, 33 bp, synthetic construct
  source 1..33: mol_type = other DNA
agtctcacaagcctggatgggtccacagatggc

SEQ ID NO: 19 - DNA, 55 bp, synthetic construct
  source 1..55: mol_type = other DNA
  modified_base 38: mod_base = OTHER; note = 5-Methylcytosine
tcggcgatctacgcagcgacgactgtacatgtcctcgcactggctcgcggtctct

SEQ ID NO: 20 - DNA, 39 bp, synthetic construct
  source 1..39: mol_type = other DNA
  modified_base 1: mod_base = OTHER; note = Phosphorylated nucleotide
tatcagagaccgcgagccagtgcgaggacatgtacagtc

SEQ ID NO: 21 - DNA, 56 bp, synthetic construct
  source 1..56: mol_type = other DNA
  modified_base 1: mod_base = OTHER; note = Phosphorylated nucleotide
gatatcatctgtggacccatccaggcttgtgagactgtagctcttcgagatcaagc

SEQ ID NO: 22 - DNA, 37 bp, synthetic construct
  source 1..37: mol_type = other DNA
agtctcacaagcctggatgggtccacagatgatatca

SEQ ID NO: 23 - DNA, 53 bp, synthetic construct; identical to SEQ ID NO: 17
  source 1..53: mol_type = other DNA

SEQ ID NO: 24 - DNA, 53 bp, synthetic construct; identical to SEQ ID NO: 17
  source 1..53: mol_type = other DNA

SEQ ID NO: 25 - DNA, 749 bp, synthetic construct
  source 1..749: mol_type = other DNA
cttcatgctggtctcagcttgatctcgaagagctacagcgccgtacagactcagtgctccttggctgatccggaatgccgccgttatccaggttgaatgaagtatcctggactgtgcgggacccagaacaaccctggtggctattctaatacagcctggaggcgtaacacctggtgcatggacttcaggctgtttcagttccttaagcacgacagcctcgtagtcagtagcctggtacgatcatactaccctgggctgaagttctttcgtagtttccactgccgtattgcagcatgataccttgtcagaagccttacgtagtcgtatcctggtacgtcctggcgtctggaaggcttgcttgcactgagatttttctcagtgcaagccttccagacgccaggacgtaccaggatacgactacgtaaggcttctgacaaggtatcatgctgcaatacggcagtggaaactacgaaagaacttcagcccagggtagtatgatcgtaccaggctactgactacgaggctgtcgtgcttaaggaactgaaacagcctgaagtccatgcaccaggtgttacgcctccaggctgtattagaatagccaccagggttgttctgggtcccgcacagtccaggatacttcattcaacctggataacggcggcattccggatcagtcaaggagcactgcatccattcacagacgtcgctgcgtagatcgccgattccacttcctaatctgtcatcttctg

SEQ ID NO: 26 - DNA, 749 bp, synthetic construct
  source 1..749: mol_type = other DNA
cttcatgctggtctcagcttgatctcgaagagctacagcgccgtacagactcagtgctccttggctgatccggaatgccgccgttatccaggttgaatgaagtatcctggactgtgctgtcgggttaagccacgtattaaccaggttaacatagtgccattatctctttatcagggcacgctctaaactccagggcttagggacgcaaagccaaggaccggcttggccaggtatcggtaaatttgcctcagatgcgatttgcaaattgaccaggttacgccggcataggctaggttgagatttggccaggctcgtacgtagtcgtatcctggtacgtcctggcgtctggaaggcttgcttgcactgagatttttctcagtgcaagccttccagacgccaggacgtaccaggatacgactacgtacgagcctggccaaatctcaacctagcctatgccggcgtaacctggtcaatttgcaaatcgcatctgaggcaaatttaccgatacctggccaagccggtccttggctttgcgtccctaagccctggagtttagagcgtgccctgataaagagataatggcactatgttaacctggttaatacgtggcttaacccgacagcacagtccaggatacttcattcaacctggataacggcggcattccggatcagtcaaggagcactgcatccattcacagacgtcgctgcgtagatcgccgattccacttcctaatctgtcatcttctg

SEQ ID NO: 27 - DNA, 749 bp, synthetic construct
  source 1..749: mol_type = other DNA
cttcatgctggtctcagcttgatctcgaagagctacagcgccgtacagactcagtgctccttggctgatccggaatgccgccgttatccaggttgaatgaagtatcctggactgaggacagcccaggcagtcgcaatacagaaattagactaactccgtgggccaccgacgttcatctggccaagtctccgccaggacttgtgttgatgtcaaggttgcacgtgaaatccatccaggccaaccagcgggcctgtggcatattcacggtggattcccaggtaacaccgaatatcgcctgggtgccctttgctacttacgtagtcgtatcctggtacgtcctggcgtctggaaggcttgcttgcactgagatttttctcagtgcaagccttccagacgccaggacgtaccaggatacgactacgtaagtagcaaagggcacccaggcgatattcggtgttacctgggaatccaccgtgaatatgccacaggcccgctggttggcctggatggatttcacgtgcaaccttgacatcaacacaagtcctggcggagacttggccagatgaacgtcggtggcccacggagttagtctaatttctgtattgcgactgcctgggctgtcctcagtccaggatacttcattcaacctggataacggcggcattccggatcagtcaaggagcactgcatccattcacagacgtcgctgcgtagatcgccgattccacttcctaatctgtcatcttctg

SEQ ID NO: 28 - DNA, 749 bp, synthetic construct; identical to SEQ ID NO: 27
  source 1..749: mol_type = other DNA

SEQ ID NO: 29 - DNA, 749 bp, synthetic construct
  source 1..749: mol_type = other DNA
cttcatgctggtctcagcttgatctcgaagagctacagcgccgtacagactcagtgctccttggctgatccggaatgccgccgttatccaggttgaatgaagtatcctggactgcctgggcctatgtaacagggtgagcgcattacggcccggtatgcccagggccactacatgagcacgatgctaacagtgacacctggtagcttagcacatccagagtgtgtagcgaccgccagggagtccgttggataggtgttgattgattagatcctggccgaatatggaaagtcggtaggtatgattgttctcgcccgtacgtagtcgtatcctggtacgtcctggcgtctggaaggcttgcttgcactgagatttttctcagtgcaagccttccagacgccaggacgtaccaggatacgactacgtacgggcgagaacaatcatacctaccgactttccatattcggccaggatctaatcaatcaacacctatccaacggactccctggcggtcgctacacactctggatgtgctaagctaccaggtgtcactgttagcatcgtgctcatgtagtggccctgggcataccgggccgtaatgcgctcaccctgttacataggcccaggcagtccaggatacttcattcaacctggataacggcggcattccggatcagtcaaggagcactgcatccattcacagacgtcgctgcgtagatcgccgattccacttcctaatctgtcatcttctg

SEQ ID NO: 30 - DNA, 746 bp, synthetic construct
  source 1..746: mol_type = other DNA
cttcatgctggtctcagcttgatctcgaagagctacagcgccgtacagactcagtgctccttggctgatccggaatgccgccgttatccaggttgaatgaagtatcctggactgatgattccatctccgttatccaggcgactttgtgcgttcatatatgatgagtaatccacgagtatattaatagatcctggcagatcctggtccagggaccctgaccgcgcgggccaggaacaggcaagctggtcaagcctggtgccgaacattgttacctagttaaaggccagtagtccctgcccaataaccttccaccatacgtagtcgtatcctggtacgtcctggcgtctggaaggcgcttgcactgagatttttctcagtgcgccttccagacgccaggacgtaccaggatacgactacgtatggtggaaggttattgggcagggactactggcctttaactaggtaacaatgttcggcaccaggcttgaccagcttgcctgttcctggcccgcgcggtcagggtccctggaccaggatctgccaggatctattaatatactcgtggattactcatcatatatgaacgcacaaagtcgcctggataacggagatggaatcatcagtccaggatacttcattcaacctggataacggcggcattcccggatcagtcaaggagcactgcatccattcacagacgtcgctgcgtagatcgccgattccacttcctaatctgtcatcttctg

SEQ ID NO: 31 - DNA, 749 bp, synthetic construct
  source 1..749: mol_type = other DNA
cttcatgctggtctcagcttgatctcgaagagctacagcgccgtacagactcagtgctccttggctgatccggaatgccgccgttatccaggttgaatgaagtatcctggactgcaagttctcagtccctggcgtactgttcagacctccgcagccgttgccaggtgggagtgcaaacaccacatacaacacgtttcggccttattccagcctggtcaacgaccagcgcctggttgtcagcgcaattccttatacccgccacctggcggctagtagacaggatattattagggccacgggccgtaagtccgtcgtacgtagtcgtatcctggtacgtcctggcgtctggaaggcttgcttgcactgagatttttctcagtgcaagccttccagacgccaggacgtaccaggatacgactacgtacgacggacttacggcccgtggccctaataatatcctgtctactagccgccaggtggcgggtataaggaattgcgctgacaaccaggcgctggtcgttgaccaggctggaataaggccgaaacgtgttgtatgtggtgtttgcactcccacctggcaacggctgcggaggtctgaacagtacgccagggactgagaacttgcagtccaggatacttcattcaacctggataacggcggcattccggatcagtcaaggagcactgcatccattcacagacgtcgctgcgtagatcgccgattccacttcctaatctgtcatcttctg

SEQ ID NO: 32 - DNA, 749 bp, synthetic construct
  source 1..749: mol_type = other DNA
cttcatgctggtctcagcttgatctcgaagagctacagcgccgtacagactcagtgctccttggctgatccggaatgccgccgttatccaggttgaatgaagtatcctggactgatcgcgaaagatttcaggaacacctccctggtggaacttagtctatgccatagttaagctaaattgccagggcagtcgtgactgcgccgtaggaacaagtccctccaagtgtgggacgcacctggtcgcaaagcacgcctggaggctactatggtttagcccagggcgttacacactaatccgcctttgttgtgtagttatacgtagtcgtatcctggtacgtcctggcgtctggaaggcttgcttgcactgagatttttctcagtgcaagccttccagacgccaggacgtaccaggatacgactacgtataactacacaacaaaggcggattagtgtgtaacgccctgggctaaaccatagtagcctccaggcgtgctttgcgaccaggtgcgtcccacacttggagggacttgttcctacggcgcagtcacgactgccctggcaatttagcttaactatggcatagactaagttccaccagggaggtgttcctgaaatctttcgcgatcagtccaggatacttcattcaacctggataacggcggcattccggatcagtcaaggagcactgcatccattcacagacgtcgctgcgtagatcgccgattccacttcctaatctgtcatcttctg

SEQ ID NO: 33 - DNA, 666 bp, synthetic construct
  source 1..666: mol_type = other DNA
gtctgtgaatggatgcagtgctccttggctgatccggaatgccgccgttatccaggttgaatgaagtatcctggactgatcgcgaaagatttcaggaacacctccctggtggaacttagtctatgccatagttaagctaaattgccagggcagtcgtgactgcgccgtaggaacaagtccctccaagtgtgggacgcacctggtcgcaaagcacgcctggaggctactatggtttagcccagggcgttacacactaatccgcctttgttgtgtagttatacgtagtcgtatcctggtacgtcctggcgtctggaaggcttgcttgcactgagaaaaaactcagtgcaagccttccagacgccaggacgtaccaggatacgactacgtataactacacaacaaaggcggattagtgtgtaacgccctgggctaaaccatagtagcctccaggcgtgctttgcgaccaggtgcgtcccacacttggagggacttgttcctacggcgcagtcacgactgccctggcaatttagcttaactatggcatagactaagttccaccagggaggtgttcctgaaatctttcgcgatcagtccaggatacttcattcaacctggataacggcggcattccggatcagccaaggagcactgagtctgtacggcgct

SEQ ID NO: 34 - DNA, 53 bp, synthetic construct
  source 1..53: mol_type = other DNA
tcggcgatctacgcagcgacgccatctgtggacccatccaggcttgtgagact

SEQ ID NO: 35 - DNA, 314 bp, synthetic construct
  source 1..314: mol_type = other DNA
ggtctctgaatctgagttgatatgtccaggtatttgtcctattatccaggttctacatggttctgtgaacaaggttgcacaaggttacgcatctcaaggttactcaaggtcaatgattgacaagcatggttctacatggtgtgtacatgcaaggtagtacctatgactacatggtgcaacatggttcttgcttgtctatcaaggtcacatatgtcaaggttacattgtctgttcacatggtcactcatatcaaggtactatagacgagttgataaccaggtgtatccaggtttcaagcattgagcttagagacc

SEQ ID NO: 36 - DNA, 132 bp, synthetic construct
  source 1..132: mol_type = other DNA
cttcatgcgctctgagtctctgcgagcgcacgtggcgacggcacgacgccttttggcgtcgtgccgtcgccacgtgcgctcgcacgtcacagctgaagcgccgtacagactcagtgctccttggctgatccg

SEQ ID NO: 37 - DNA, 144 bp, synthetic construct
  source 1..144: mol_type = other DNA
  modified_base 1: mod_base = OTHER; note = Phosphorylated nucleotide
attccggatcagccaaggagcactgcatccattcacagacgtcgctgactgttgcgagcgcacgtggcgacggcacgacgccttttggcgtcgtgccgtcgccacgtgcgctcgcacagactcgctcgatctgtcatcttctgt

SEQ ID NO: 38 - DNA, 694 bp, synthetic construct
  source 1..694: mol_type = other DNA
gtctgtgaatggatgcagtgctccttggctgatccggaatctgagttgatatgtccaggtatttgtcctattatccaggttctacatggatctgtgaacaaggttgcacaaggttacgcatctcaaggatactcaaggtcaatgattgacaagcatggttctacatggagtgtacatgcaaggtagtacctatgactacatggagcaacatggttcttgcttgtctatcaaggactcatatgtcaaggttacattgtctgttcacatggactctcatatcaaggtactatagacgagttgataaccaggtgtatccaggattcaagcattgagcttgcactgagaaaaatctcagtgcaagctcaatgcttgaatcctggatacacctggttatcaactcgtctatagtaccttgatatgagagtccatgtgaacagacaatgtaaccttgacatatgagtccttgatagacaagcaagaaccatgttgctccatgtagtcataggtactaccttgcatgtacactccatgtagaaccatgcttgtcaatcattgaccttgagtatccttgagatgcgtaaccttgtgcaaccttgttcacagatccatgtagaacctggataataggacaaatacctggacatatcaactcagattccggatcagccaaggagcactgagtctgtacggcgct

SEQ ID NO: 39 - DNA, 902 bp, synthetic construct
  source 1..902: mol_type = other DNA
cttcatgcgctctgagtctctgcgagcgcacgtggcgacggcacgacgccttttggcgtcgtgccgtcgccacgtgcgctcgcacgtcacagctgaagcgccgtacagactcagtgctccttggctgatccggaatctgagttgatatgtccaggtatttgtcctattatccaggttctacatggatctgtgaacaaggttgcacaaggttacgcatctcaaggatactcaaggtcaatgattgacaagcatggttctacatggagtgtacatgcaaggtagtacctatgactacatggagcaacatggttcttgcttgtctatcaaggactcatatgtcaaggttacattgtctgttcacatggactctcatatcaaggtactatagacgagttgataaccaggtgtatccaggattcaagcattgagcttgcttgcactgagatttttctcagtgcaagcaagctcaatgcttgaatcctggatacacctggttatcaactcgtctatagtaccttgatatgagagtccatgtgaacagacaatgtaaccttgacatatgagtccttgatagacaagcaagaaccatgttgctccatgtagtcataggtactaccttgcatgtacactccatgtagaaccatgcttgtcaatcattgaccttgagtatccttgagatgcgtaaccttgtgcaaccttgttcacagatccatgtagaacctggataataggacaaatacctggacatatcaactcagattccggatcagccaaggagcactgcatccattcacagacgtcgctgactgttgcgagcgcacgtggcgacggcacgacgccttttggcgtcgtgccgtcgccacgtgcgctcgcacagactcgctcgatctgtcatcttctgt

SEQ ID NO: 40 - DNA, 894 bp, synthetic construct
  source 1..894: mol_type = other DNA
cttcatgcgctctgagtctctgcgagcgcacgtggcgacggcacgacgccttttggcgtcgtgccgtcgccacgtgcgctcgcacgtcacagctgaagcgccgtacagactcagtgctccttggctgatccggaatctgagttgatatgtccaggtatttgtcctattatccaggttctacctggatctgtgaaccaggttgcaccaggttacgcatctcaaggatactcaaggtcaatgattgacaagcatggttctacatggagtgtacatgcaaggtagtacctatgactacatggagcaacatggttcttgcttgtctatcaaggactcatatgtcaaggttacattgtctgttcacatggactctcatatcaaggtactatagacgagttgataaccaggtgtatccaggattcaagcattgagcttgcactgagatttttctcagtgcaagctcaatgcttgaatcctggatacacctggttatcaactcgtctatagtaccttgatatgagagtccatgtgaacagacaatgtaaccttgacatatgagtccttgatagacaagcaagaaccatgttgctccatgtagtcataggtactaccttgcatgtacactccatgtagaaccatgcttgtcaatcattgaccttgagtatccttgagatgcgtaacctggtgcaacctggttcacagatccaggtagaacctggataataggacaaatacctggacatatcaactcagattccggatcagccaaggagcactgcatccattcacagacgtcgctgactgttgcgagcgcacgtggcgacggcacgacgccttttggcgtcgtgccgtcgccacgtgcgctcgcacagactcgctcgatctgtcatcttctgt

SEQ ID NO: 41 - DNA, 894 bp, synthetic construct
  source 1..894: mol_type = other DNA
cttcatgcgctctgagtctctgcgagcgcacgtggcgacggcacgacgccttttggcgtcgtgccgtcgccacgtgcgctcgcacgtcacagctgaagcgccgtacagactcagtgctccttggctgatccggaatctgagttgatatgtccaggtatttgtcctattatccaggttctacctggatctgtgaacaaggttgcacaaggttacgcatctccaggatactcaaggtcaatgattgacaagcatggttctacctggagtgtacatgcaaggtagtacctatgactacatggagcaacctggttcttgcttgtctatcaaggactcatatgtcaaggttacattgtctgttcacatggactctcatatcaaggtactatagacgagttgataaccaggtgtatccaggattcaagcattgagcttgcactgagatttttctcagtgcaagctcaatgcttgaatcctggatacacctggttatcaactcgtctatagtaccttgatatgagagtccatgtgaacagacaatgtaaccttgacatatgagtccttgatagacaagcaagaaccaggttgctccatgtagtcataggtactaccttgcatgtacactccaggtagaaccatgcttgtcaatcattgaccttgagtatcctggagatgcgtaaccttgtgcaaccttgttcacagatccaggtagaacctggataataggacaaatacctggacatatcaactcagattccggatcagccaaggagcactgcatccattcacagacgtcgctgactgttgcgagcgcacgtggcgacggcacgacgccttttggcgtcgtgccgtcgccacgtgcgctcgcacagactcgctcgatctgtcatcttctgt

SEQ ID NO: 42 - DNA, 894 bp, synthetic construct
  source 1..894: mol_type = other DNA
cttcatgcgctctgagtctctgcgagcgcacgtggcgacggcacgacgccttttggcgtcgtgccgtcgccacgtgcgctcgcacgtcacagctgaagcgccgtacagactcagtgctccttggctgatccggaatctgagttgatatgtccaggtatttgtcctattatccaggttctacctggatctgtgaaccaggttgcacaaggttacgcatctccaggatactccaggtcaatgattgacaagcctggttctacatggagtgtacatgcaaggtagtacctatgactacatggagcaacatggttcttgcttgtctatcaaggactcatatgtcaaggttacattgtctgttcacatggactctcatatcaaggtactatagacgagttgataaccaggtgtatccaggattcaagcattgagcttgcactgagatttttctcagtgcaagctcaatgcttgaatcctggatacacctggttatcaactcgtctatagtaccttgatatgagagtccatgtgaacagacaatgtaaccttgacatatgagtccttgatagacaagcaagaaccatgttgctccatgtagtcataggtactaccttgcatgtacactccatgtagaaccaggcttgtcaatcattgacctggagtatcctggagatgcgtaaccttgtgcaacctggttcacagatccaggtagaacctggataataggacaaatacctggacatatcaactcagattccggatcagccaaggagcactgcatccattcacagacgtcgctgactgttgcgagcgcacgtggcgacggcacgacgccttttggcgtcgtgccgtcgccacgtgcgctcgcacagactcgctcgatctgtcatcttctgt

SEQ ID NO: 43 - DNA, 894 bp, synthetic construct
  source 1..894: mol_type = other DNA
cttcatgcgctctgagtctctgcgagcgcacgtggcgacggcacgacgccttttggcgtcgtgccgtcgccacgtgcgctcgcacgtcacagctgaagcgccgtacagactcagtgctccttggctgatccggaatctgagttgatatgtccaggtatttgtcctattatccaggttctacctggatctgtgaaccaggttgcacaaggttacgcatctccaggatactcaaggtcaatgattgacaagcatggttctacctggagtgtacatgcaaggtagtacctatgactacatggagcaacatggttcttgcttgtctatccaggactcatatgtcaaggttacattgtctgttcacctggactctcatatcaaggtactatagacgagttgataaccaggtgtatccaggattcaagcattgagcttgcactgagatttttctcagtgcaagctcaatgcttgaatcctggatacacctggttatcaactcgtctatagtaccttgatatgagagtccaggtgaacagacaatgtaaccttgacatatgagtcctggatagacaagcaagaaccatgttgctccatgtagtcataggtactaccttgcatgtacactccaggtagaaccatgcttgtcaatcattgaccttgagtatcctggagatgcgtaaccttgtgcaacctggttcacagatccaggtagaacctggataataggacaaatacctggacatatcaactcagattccggatcagccaaggagcactgcatccattcacagacgtcgctgactgttgcgagcgcacgtggcgacggcacgacgccttttggcgtcgtgccgtcgccacgtgcgctcgcacagactcgctcgatctgtcatcttctgt

SEQ ID NO: 44 - DNA, 894 bp, synthetic construct
  source 1..894: mol_type = other DNA
cttcatgcgctctgagtctctgcgagcgcacgtggcgacggcacgacgccttttggcgtcgtgccgtcgccacgtgcgctcgcacgtcacagctgaagcgccgtacagactcagtgctccttggctgatccggaatctgagttgatatgtccaggtatttgtcctattatccaggttctacatggatctgtgaacaaggttgcacaaggttacgcatctcaaggatactcaaggtcaatgattgacaagcatggttctacctggagtgtacatgccaggtagtacctatgactacctggagcaacatggttcttgcttgtctatcaaggactcatatgtcaaggttacattgtctgttcacatggactctcatatcaaggtactatagacgagttgataaccaggtgtatccaggattcaagcattgagcttgcactgagatttttctcagtgcaagctcaatgcttgaatcctggatacacctggttatcaactcgtctatagtaccttgatatgagagtccatgtgaacagacaatgtaaccttgacatatgagtccttgatagacaagcaagaaccatgttgctccaggtagtcataggtactacctggcatgtacactccaggtagaaccatgcttgtcaatcattgaccttgagtatccttgagatgcgtaaccttgtgcaaccttgttcacagatccatgtagaacctggataataggacaaatacctggacatatcaactcagattccggatcagccaaggagcactgcatccattcacagacgtcgctgactgttgcgagcgcacgtggcgacggcacgacgccttttggcgtcgtgccgtcgccacgtgcgctcgcacagactcgctcgatctgtcatcttctgt

SEQ ID NO: 45 - DNA, 53 bp, synthetic construct; identical to SEQ ID NO: 34
  source 1..53: mol_type = other DNA

SEQ ID NO: 46 - DNA, 53 bp, synthetic construct
  source 1..53: mol_type = other DNA
gccatctgtggacccatccaggcttgtgagactgtcacacgatatctcagcta

SEQ ID NO: 47 - DNA, 72 bp, synthetic construct
  source 1..72: mol_type = other DNA
cttcatgctggtctcatagctgagatatcgtgtgacagcgccgtacagactcagtgctccttggctgatccg

SEQ ID NO: 48 - DNA, 745 bp, synthetic construct
  source 1..745: mol_type = other DNA
cttcatgctggtctcagcttgatctcgaagagctacagcgccgtacagactcagtgctccttggctgatccggaatgccgccgttatccaggttgaatgaagtatcctggactgatcgcgaaagatttcaggaacacctccctggtggaacttagtctatgccatagttaagctaaattgcaagggcagtcgtgactgcgccgtaggaacaagtccctccaagtgtgggacgcacatggtcgcaaagcacgcatggaggctactatggtttagcccagggcgttacacactaatccgcctttgttgtgtagttatacgtagtcgtatcctggtacgtcctggcgtctggaaggcttgcactgagatttttctcagtgcaagccttccagacgccaggacgtaccaggatacgactacgtataactacacaacaaaggcggattagtgtgtaacgccctgggctaaaccatagtagcctccatgcgtgctttgcgaccatgtgcgtcccacacttggagggacttgttcctacggcgcagtcacgactgcccttgcaatttagcttaactatggcatagactaagttccaccagggaggtgttcctgaaatctttcgcgatcagtccaggatacttcattcaacctggataacggcggcattccggatcagccaaggagcactgcatccattcacagacgtcgctgcgtagatcgccgattccacttcctaatctgtcatcttctg

SEQ ID NO: 49 - DNA, 745 bp, synthetic construct
  source 1..745: mol_type = other DNA
cttcatgctggtctcatagctgagatatcgtgtgacagcgccgtacagactcagtgctccttggctgatccggaatgccgccgttatccaggttgaatgaagtatcctggactgatcgcgaaagatttcaggaacacctccatggtggaacttagtctatgccatagttaagctaaattgcaagggcagtcgtgactgcgccgtaggaacaagtccctccaagtgtgggacgcacctggtcgcaaagcacgcctggaggctactatggtttagcccagggcgttacacactaatccgcctttgttgtgtagttatacgtagtcgtatcctggtacgtcctggcgtctggaaggcttgcactgagatttttctcagtgcaagccttccagacgccaggacgtaccaggatacgactacgtataactacacaacaaaggcggattagtgtgtaacgccctgggctaaaccatagtagcctccaggcgtgctttgcgaccaggtgcgtcccacacttggagggacttgttcctacggcgcagtcacgactgcccttgcaatttagcttaactatggcatagactaagttccaccatggaggtgttcctgaaatctttcgcgatcagtccaggatacttcattcaacctggataacggcggcattccggatcagccaaggagcactgcatccattcacagacgtcgctgcgtagatcgccgattccacttcctaatctgtcatcttctg

SEQ ID NO: 50 - DNA, 88 bp, synthetic construct
  source 1..88: mol_type = other DNA
  misc_difference 1..12: note = Any nucleotide
  misc_difference 77..88: note = Any nucleotide
nnnnnnnnnnnntgcgagcgcacgtggcgacggcacgacgccttttggcgtcgtgccgtcgccacgtgcgctcgcannnnnnnnnnnn

SEQ ID NO: 51 - DNA, 106 bp, synthetic construct
  source 1..106: mol_type = other DNA
  misc_difference 19..30: note = Any nucleotide
  misc_difference 95..106: note = Any nucleotide
gtgactgagtcagagtttnnnnnnnnnnnntgcgagcgcacgtggcgacggcacgacgccaaaaggcgtcgtgccgtcgccacgtgcgctcgcannnnnnnnnnnn

SEQ ID NO: 52 - DNA, 106 bp, synthetic construct
  source 1..106: mol_type = other DNA
  misc_difference 1..12: note = Any nucleotide
  misc_difference 77..88: note = Any nucleotide
nnnnnnnnnnnntgcgagcgcacgtggcgacggcacgacgccaaaaggcgtcgtgccgtcgccacgtgcgctcgcannnnnnnnnnnntttgtgactgagtcagag

SEQ ID NO: 53 - DNA, 314 bp, synthetic construct
  source 1..314: mol_type = other DNA
ggtctctgaatctgagttgatatgtccaggtatttgtcctattatccaggttctacatggttctgtgaacaaggttgcacacggttacgcatctcaaggttactcaaggtcaatgattgacaagcatggttctacatggtgtgtacatgcaaggtagtacctatgactacatggtgcaacatggttcttgcttgtctatcaaggtcacatatgtcaaggttacattgtctgttcacatggtcactcatatcaaggtactatagacgagttgataaccaggtgtatccaggtttcaagcattgagcttagagacc

SEQ ID NO: 54 - skipped

SEQ ID NO: 55 - DNA, 46 bp, synthetic construct
  source 1..46: mol_type = other DNA
attccggatcagccaaggagcactgcagtgctccttggctgatccg

SEQ ID NO: 56 - DNA, 66 bp, synthetic construct
  source 1..66: mol_type = other DNA
cttcatgctggtctcagcttgatctcgaagagctacagcgccgtacagactagtctgtacggcgct

SEQ ID NO: 57 - DNA, 68 bp, synthetic construct
  source 1..68: mol_type = other DNA
gtctgtgaatggatgcatccattcacagacattgcttggtaggggcgtgctttaatgcccccattcaa

SEQ ID NO: 58 - DNA, 46 bp, synthetic construct; identical to SEQ ID NO: 55
  source 1..46: mol_type = other DNA

(19) United States
(12) Patent Application Publication
(10) Pub. No.: US 2026/0258402 A1
(43) Pub. Date: Sep. 3, 2026

(54) DEVICE FOR DETERMINING MOLECULAR INTERACTIONS

(71) Applicant: Depixus SAS, Paris (FR)

(72) Inventor: Jimmy OUELLET, Lardy (FR)

(21) Appl. No.: 19/545,282

(22) Filed: Feb. 20, 2026

Related U.S. Application Data
(63) Continuation of application No. PCT/IB2024/058149, filed on Aug. 21, 2024.
(60) Provisional application No. 63/520,896, filed on Aug. 21, 2023.

(51) Int. Cl. C12N 15/10

(52) U.S. Cl. CPC C12N 15/1065

(57) ABSTRACT

Disclosed herein are methods of determining binding interactions between two or more candidate molecules. Also, disclosed herein are methods of determining binding kinetics for two or more candidate molecules. Also, disclosed herein are methods for screening two candidate molecules having binding interactions with each other.

This application is a continuation of International Application No. PCT/IB2024/058149, filed Aug. 21, 2024, which claims the benefit of priority to U.S. Provisional Application No. 63/520,896, filed on Aug. 21, 2023 the entire contents of each of which are incorporated herein by reference.

### INCORPORATION BY REFERENCE OF SEQUENCE LISTING

The instant application contains a Sequence Listing, which has been submitted via Patent Center. The Sequence Listing titled 201326-702601_PCT_SL.xml, which was created on Aug. 19, 2024, and is 76,037 bytes in size, is hereby incorporated by reference in its entirety.

### BACKGROUND

Candidates that can bind to target molecule can be identified by high throughput screening. High throughput screening allows to test thousands to millions of molecules using automated equipment. To identify candidates, one strategy consists of identifying low quality hits first based on fragment libraries consisting of simple scaffold chemical structures, and then affine the selection for example, by modifying high quality hits to design candidates. In parallel, it is also possible to test various doses of selected candidates to reach a quantitative analysis of molecules' behavior and potentially predict their activity.

Attempts have been made to explore these molecular interactions, which usually occur at low forces, by either using fluorescence and some complicated system composed of a dsDNA leach but these systems are difficult to put in place and cannot be scaled to multiple interactions. Moreover, modifying proteins or molecules with fluorophores in general introduces a risk that they become inactive or the interaction with other molecules can be disturbed. As it requires a lot of manipulation to attach the binding molecules to the system, this is not possible to make the system universal and to high throughput to test different interactions in parallel.

To improve the selection of these potential candidates, and/or to get more meaningful results, several methods have been developed such as the very sensitive optical method “Surface Plasmon Resonance (SPR)”. SPR occurs when polarized light strikes an electrically conducting surface at the interface between two media. This generates electron charge density waves called plasmons, reducing the intensity of reflected light at a specific angle known as the resonance angle, in proportion to the mass on a sensor surface. This technique allows therefore to detect molecular interactions in real time. However, this still relies on the binding between molecules only. Attempts have been made to identify methods to improve the accuracy for selecting candidates, but these methods remain challenging. Therefore, there is still a need for devices and methods for a faster, more selective and accurate detection of a new candidate acting on a nucleic acid or a protein.

### SUMMARY

Provided herein are nucleic acid scaffolds for determining a binding interaction between a first candidate molecule and a second candidate molecule. In some embodiments, the nucleic acid scaffold comprises: (a) a contiguous polynucleotide sequence comprising: (i) a first end that is attached to a bead, wherein the first end comprises a first molecule binding sequence, (ii) a second end that is attached to a bottom surface of a device, wherein the second end comprises a second molecule binding sequence, and (iii) an intermediate portion between the first molecule binding sequence of the first end and the second molecule binding sequence of the second end, wherein the intermediate portion comprises: (I) a first pin forming sequence comprising a barcode, (II) a second pin forming sequence that is complementary to the first pin forming sequence, wherein the first pin forming sequence is hybridized to the second pin forming sequence, and (III) a loop linking the first pin forming sequence and the second pin forming sequence; (b) a first spacer polynucleotide that has a polynucleotide sequence that is complementary to the first pin forming sequence; and (c) a second spacer polynucleotide that has a polynucleotide sequence that is complementary to the second pin forming sequence, wherein the first spacer polynucleotide and the second spacer polynucleotide are hybridized to the intermediate portion, and wherein the nucleic acid scaffold requires a force of applied to the bead along an axis perpendicular to the bottom surface of the device to unfold the contiguous polynucleotide sequence along the axis to form the nucleic acid scaffold. In some embodiments, the force can be of 0.1 pN to 10 pN. In some embodiments, the barcode sequence comprises one or more modified nucleotides. In some embodiments, the intermediate portion further comprises a first junction sequence and a second junction sequence, wherein the first junction sequence is located between the first molecule binding sequence and the first pin forming sequence, and wherein the second junction sequence is located between the second molecule binding sequence and the second pin forming sequence, and wherein the first junction sequence and the second junction sequence are not complementary to each other. In some embodiments, the first spacer polynucleotide and the second spacer polynucleotide are linked to each other by a spacer loop sequence, wherein the spacer loop sequence is hybridized to the loop. In some embodiments, each, the first molecule binding sequence and the second molecule binding sequence, independently comprises a small hairpin nucleic acid having a size in a range of from 5 bases to 100 bases.

Also provided herein are screening nucleic acid scaffolds. In some embodiments, the screening nucleic acid scaffolds comprise: (a) any one of the nucleic acid scaffolds described herein; (b) a first candidate molecule linked to a first glue sequence, wherein the first glue sequence is hybridized to the first molecule binding sequence; and (c) a second candidate molecule linked to a second glue sequence, wherein the second glue sequence is hybridized to the second molecule binding sequence.

Also provided herein are devices comprising: (a) a chamber disposed within the device, wherein the chamber comprises a bottom surface capable of immobilizing a nucleic acid scaffold; (b) a force application mechanism for stretching the contiguous polynucleotide sequence along the axis to form the nucleic acid scaffold; and (c) any one of the nucleic acid scaffolds described herein or the screening nucleic acid scaffold described herein.

Also provided herein are methods of determining a binding interaction between a first candidate molecule and a second candidate molecule. In some embodiments, the methods comprise: (a) providing a nucleic acid scaffold described herein, wherein the nucleic acid scaffold is positioned along an axis perpendicular to a bottom surface of the chamber of the device; (b) providing a device that comprises: (i) a chamber disposed within the device, wherein the chamber comprises a bottom surface capable of immobilizing a nucleic acid scaffold, and (ii) a force application mechanism for stretching the contiguous polynucleotide sequence along the axis to form the nucleic acid scaffold; (c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence; (d) determining a reference elongation length of the nucleic acid scaffold in response to a force in the absence of the first candidate molecule and the second candidate molecule in real time by: (i) applying a force of 0.1 to 50 pN to the bead attached to the nucleic acid scaffold via the force application mechanism along the axis perpendicular to the bottom surface of the device, wherein the nucleic acid scaffold is configured to unfold in response to the force applied, thereby resulting in a change in position of the bead attached to the nucleic acid scaffold along the axis, and (ii) measuring the change in position of the bead along the axis via a sensor, thereby determining the reference elongation length of the nucleic acid scaffold in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity with each other; (f) determining an elongation length of the screening nucleic acid scaffold in response to the same force used in the absence of the first candidate molecule and the second candidate molecule by repeating (d); and (g) calculating a differential value, wherein the differential value is a difference between the elongation length of the screening nucleic acid scaffold and the reference elongation length at the force applied, whereby a non-zero differential value indicates presence of the binding interaction between the first candidate molecule and the second molecule at the force applied, and whereby differential value of zero indicates absence of the binding interaction between the first candidate molecule and the second candidate molecule at the force applied. In some embodiments, the methods further comprise determining binding energy (enthalpy, entropy and AG) and thereby, optionally, determining binding kinetics of the binding interaction between the first candidate molecule and the second candidate molecule by: (h) removing the force applied by the force application mechanism to the bead after (g), thereby resulting in relaxation of the screening nucleic acid scaffold; and (i) repeating (f)-(h) and calculating a difference between the elongation length of the screening nucleic acid scaffold and the reference elongation length as a function of time. In some embodiments, the methods further comprise: (i) determining a Kon for the binding of the second molecule to the first candidate molecule, wherein the Kon is calculated based on the number of cycles of repeating (f)-(h) that result in the differential between the elongation length of the screening nucleic acid scaffold and the reference elongation length. In some embodiments, the methods further comprise: (h) determining a Koff for the binding of the second molecule to the first candidate molecule, wherein the Koff is calculated based on the length of time that the differential between the elongation length of the screening nucleic acid scaffold and the reference elongation length is present during each cycle of repeating (f)-(h).

Also provided herein are methods of determining a binding interaction between a first candidate molecule, a second candidate molecule and a third candidate molecule. In some embodiments, the methods comprise: (a) providing a nucleic acid scaffold described herein, wherein the nucleic acid scaffold is positioned along an axis perpendicular to a bottom surface of the chamber of the device; (b) providing a device that comprises: (i) a chamber disposed within the device, wherein the chamber comprises a bottom surface, and (ii) a force application mechanism for stretching the contiguous polynucleotide sequence along the axis to form the nucleic acid scaffold; (c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence; (d) determining a reference elongation length of the nucleic acid scaffold in the absence of the first candidate molecule and the second candidate molecule by: (i) applying a force of 0.1 pN to 50 pN to the bead attached to the nucleic acid scaffold via the force application mechanism along the axis perpendicular to the bottom surface of the chamber of the device, wherein the nucleic acid scaffold is configured to unfold in response to the force applied, thereby resulting in a change in position of the bead attached to the nucleic acid scaffold along the axis, and (ii) measuring the change in position of the bead along the axis via a sensor, thereby determining the reference elongation length of the nucleic acid scaffold in the absence of the first candidate molecule and the second candidate molecule; (e) contacting the nucleic acid scaffold with the first candidate molecule and the second candidate molecule, and, thereby, forming a screening nucleic acid scaffold, wherein the first candidate molecule and the second candidate molecule are positioned along the screening nucleic acid scaffold such that the first candidate molecule and second candidate molecule are in close proximity with each other; determining a force required to achieve the same elongation length for the screening nucleic acid scaffold as the reference elongation length by repeating (d), wherein the force required is in a range of from 0.1 pN to 50 pN; (e) contacting the third candidate molecule to the screening nucleic acid scaffold; and (f) determining an elongation length of the screening nucleic acid scaffold in the presence of the third candidate molecule and in response to the same force applied in (f) by repeating (d); and (g) calculating a differential value, wherein the differential value is a difference between the elongation length of the screening nucleic acid scaffold in the presence and absence of the third candidate molecule, whereby a non-zero differential value indicates presence of a binding interaction between the third candidate molecule, and the first candidate molecule and the second candidate molecule at the force applied, and whereby a differential value of zero indicates absence of the binding interaction between: (A) the third candidate molecule and the first candidate molecule, (B) the third candidate molecule and the second candidate molecule, or (C) the third candidate molecule, and the first candidate molecule and the second candidate molecule, at the force applied. In some embodiments, the methods further comprise determining binding energy (enthalpy, entropy and ΔG) and thereby, optionally, determining binding kinetics, of the binding interaction between the first candidate molecule, the second candidate molecule and the third candidate molecule by: (j) removing the force applied by the force application mechanism to the bead after (i), thereby resulting in relaxation of the test screening nucleic acid scaffold; and (k) repeating (e)-(j) and calculating a difference between the elongation length of the screening nucleic acid scaffold in the presence and absence of the third candidate molecule as a function of time. In some embodiments, the methods further comprise: (j) determining a Kon for the binding of the third candidate molecule to the first and second molecules, wherein the Kon is calculated based on the number of cycles of repeating (h)-(j) that result in the differential between the elongation length of the screening nucleic acid scaffold in the presence and absence of the third candidate molecule. In some embodiments, the methods further comprise: (j) determining a Koff for the binding of the third candidate molecule to the first and second candidate molecules, wherein the Koff is calculated based on the length of time that the differential between the elongation length of the screening nucleic acid scaffold in the presence and absence of the third candidate molecule is present during each cycle of repeating (h)-(j).

Also provided herein are methods of screening binding interactions between a first candidate molecule, a second candidate molecule and a plurality of third candidate molecules, the method comprising: (a) providing a device that comprises: (i) a chamber disposed within the device, wherein the chamber comprises a bottom surface, (ii) a force application mechanism for stretching the contiguous polynucleotide sequence along the axis to form the nucleic acid scaffold, and (iii) a nucleic acid scaffold positioned along an axis perpendicular to the bottom surface of the chamber of the device, wherein the nucleic acid scaffold comprises the nucleic acid scaffold described herein, wherein the nucleic acid scaffold is linked to a bead at one end and a feature of the bottom surface of the chamber of the device at the other end; (b) determining a reference elongation length of the nucleic acid scaffold in the absence of the first candidate molecule and the second candidate molecules by: (iv) applying a force of 0.1 pN to 50 pN to the bead attached to the nucleic acid scaffold via the force application mechanism along the axis perpendicular to the bottom surface of the chamber of the device, wherein the nucleic acid scaffold is configured to unfold in response to the force applied, thereby resulting in a change in position of the bead attached to the nucleic acid scaffold along the axis, and (v) measuring the change in position of the bead along the axis via a sensor, thereby determining the reference elongation length for the nucleic acid scaffold in the absence of the first candidate molecule and the plurality of second candidate molecules; (c) contacting the nucleic acid scaffold with the first candidate molecule linked to a first glue sequence, thereby anchoring the first candidate molecule to the nucleic acid scaffold; (d) contacting the nucleic acid scaffold with the second candidate molecule linked to a second glue sequence, thereby anchoring the second candidate molecule to the nucleic acid scaffold, thereby forming a screening nucleic acid scaffold, wherein the first candidate molecule and the second candidate molecule of the screening nucleic acid scaffold are positioned such that the first candidate molecule and the second candidate molecule are in close proximity with each other; (e) determining a force required to achieve the same elongation length for the screening nucleic acid scaffold as the reference elongation length by repeating (d), wherein the force is in a range of from 0.1 pN to 50 pN; (f) contacting the plurality of third candidate molecules to the screening nucleic acid scaffold; (g) determining elongation lengths for the screening nucleic acid scaffold in the presence of at least one of the plurality of third candidate molecules in response to the same force applied in (e) by repeating (b); and (h) calculating a difference between the reference elongation length and the elongation length of screening nucleic acid scaffold, whereby a non-zero differential value indicates presence of a binding interaction between the third candidate molecule, and the first candidate molecule and the second candidate molecule at the force applied, and whereby a differential value of zero indicates absence of the binding interaction between: (A) the third candidate molecule and the first candidate molecule, (B) the third candidate molecule and the second candidate molecule, or (C) the third candidate molecule, and the first candidate molecule and the second candidate molecule, at the force applied.

Also provided herein are methods of screening binding interactions between a first candidate molecule and a plurality of second candidate molecules. In some embodiments, the methods comprise: (a) providing a device that comprises: (i) a chamber disposed within the device, wherein the chamber comprises a bottom surface, (ii) a force application mechanism for stretching the contiguous polynucleotide sequence along the axis to form the nucleic acid scaffold, and (iii) a plurality of nucleic acid scaffolds positioned along an axis perpendicular to the bottom surface of the chamber of the device, wherein each nucleic acid scaffold among the plurality of nucleic acid scaffolds comprises any one of the nucleic acid scaffolds described herein, wherein each of the plurality of nucleic acid scaffolds are linked to a bead at one end and a feature of the bottom surface of the chamber of the device at the other end; (b) determining a reference elongation length of each of the nucleic acid scaffold among the plurality of nucleic acid scaffolds in the absence of the first candidate molecule and the plurality of second candidate molecules by: (i) applying a force of 0.1 pN to 50 pN to the bead attached to each of the plurality of nucleic acid scaffolds via the force application mechanism along the axis perpendicular to the bottom surface of the chamber of the device, wherein each of the plurality of nucleic acid scaffolds is configured to unfold in response to the force applied, thereby resulting in a change in position of the bead attached to each of the plurality of nucleic acid scaffolds along the axis, and (ii) measuring the change in position of the bead along the axis via a sensor, thereby determining reference elongation lengths for each of the plurality of nucleic acid scaffolds in the absence of the first candidate molecule and the plurality of second candidate molecules; (c) contacting the plurality of nucleic acid scaffolds with the first candidate molecule linked to a first glue sequence, thereby anchoring the first candidate molecule to each of the plurality of nucleic acid scaffolds, wherein the first glue sequence is complementary to the first molecule binding sequence; (d) contacting the plurality of nucleic acid scaffolds with the plurality of second candidate molecules each linked to a second glue sequence, thereby anchoring one of the second candidate molecule among the plurality of candidate molecules to each of the plurality of nucleic acid scaffolds, wherein the second glue sequence is complementary to the second molecule binding sequence, thereby forming a plurality of screening nucleic acid scaffolds, wherein the first candidate molecule and the second candidate molecule of each of the plurality of screening nucleic acid scaffolds are positioned such that the first candidate molecule and the second candidate molecule are in close proximity with each other; (e) determining elongation lengths for each of the plurality of screening nucleic acid scaffolds in response to the force of 0.1 pN to 50 pN by repeating (d); and (f) calculating a difference between the reference elongation length and the elongation length of each screening nucleic acid scaffold among the plurality of screening nucleic acid scaffolds, whereby a differential between the elongation length of a screening nucleic acid scaffold and the reference elongation length indicates that the first candidate molecule and the second candidate molecule anchored to the screening nucleic acid have a binding interaction to each other at the force applied, and whereby the absence of the differential indicates absence of binding interaction between the first candidate molecule and the second candidate molecule at the force applied.

Provided herein are methods of screening binding interactions between a first candidate molecule, a second candidate molecule and a plurality of third candidate molecules. In some embodiments, the methods comprise: (a) providing a device that comprises: (i) a chamber disposed within the device, wherein the chamber comprises a bottom surface, (ii) a force application mechanism for stretching the contiguous polynucleotide sequence along the axis to form the nucleic acid scaffold, and (iii) a plurality of nucleic acid scaffolds positioned along an axis perpendicular to the bottom surface of the chamber of the device, wherein each nucleic acid scaffold among the plurality of nucleic acid scaffolds comprises any one of the nucleic acid scaffolds described herein, wherein each of the plurality of nucleic acid scaffolds are linked to a bead at one end and a feature of the bottom surface of the chamber of the device at the other end; (b) determining a reference elongation length of each of the nucleic acid scaffold among the plurality of nucleic acid scaffolds in the absence of the first candidate molecule and the second candidate molecules by: (i) applying a force of 0.1 pN to 50 pN to the bead attached to each of the plurality of nucleic acid scaffolds via the force application mechanism along the axis perpendicular to the bottom surface of the chamber of the device, wherein each of the plurality of nucleic acid scaffolds is configured to unfold in response to the force applied, thereby resulting in a change in position of the bead attached to each of the plurality of nucleic acid scaffolds along the axis, and (ii) measuring the change in position of the bead along the axis via a sensor, thereby determining reference elongation lengths for each of the plurality of nucleic acid scaffolds in the absence of the first candidate molecule and the plurality of second candidate molecules; (c) contacting the plurality of nucleic acid scaffolds with the first candidate molecule linked to a first glue sequence, thereby anchoring the first candidate molecule to each of the plurality of nucleic acid scaffolds; (d) contacting the plurality of nucleic acid scaffolds with the plurality of second candidate molecules each linked to a second glue sequence, thereby anchoring the second candidate molecule to each of the plurality of nucleic acid scaffolds, thereby forming a plurality of screening nucleic acid scaffolds, wherein the first candidate molecule and the second candidate molecule of each of the plurality of screening nucleic acid scaffolds are positioned such that the first candidate molecule and the second candidate molecule are in close proximity with each other; (e) determining a force required to achieve the same elongation length for the screening nucleic acid scaffold as the reference elongation length by repeating (d), wherein the force is in a range of from 0.1 pN to 50 pN; (f) contacting the plurality of third candidate molecules to the plurality of screening nucleic acid scaffolds; (g) determining elongation lengths for each of the plurality of screening nucleic acid scaffolds in the presence of at least one of the plurality of third candidate molecules in response to the same force applied in (e) by repeating (b); and (h) calculating a difference between the reference elongation length and the elongation length of each screening nucleic acid scaffold among the plurality of screening nucleic acid scaffolds, whereby a non-zero differential value indicates presence of a binding interaction between the third candidate molecule, and the first candidate molecule and the second candidate molecule at the force applied, and whereby a differential value of zero indicates absence of the binding interaction between: (A) the third candidate molecule and the first candidate molecule, (B) the third candidate molecule and the second candidate molecule, or (C) the third candidate molecule, and the first candidate molecule and the second candidate molecule, at the force applied. In some embodiments, at least two of the nucleic acid scaffolds comprise barcode sequences that are located at non-identical positions relative to each other. In some embodiments, at least two of the nucleic acid scaffolds comprise a barcode sequence that are non-identical relative to each other. In some embodiments, the methods further comprise determining identity of the contiguous polynucleotide sequence (e.g., hairpin nucleic acid) based on the barcode prior to (b). In some embodiments, the identity of the contiguous polynucleotide sequence (e.g., hairpin nucleic acid) is determined by detecting the position of the one or more modified nucleotides in the barcode.

Also provided herein are methods of determining a binding interaction between a first candidate molecule and a second candidate molecule, the methods comprise: (a) providing a nucleic acid scaffold described herein, wherein the nucleic acid scaffold is positioned along an axis perpendicular to a bottom surface of the chamber of the device; (b) providing a device that comprises: (i) a chamber disposed within the device, wherein the chamber comprises a bottom surface, and (ii) a force application mechanism for stretching the contiguous polynucleotide sequence along the axis to form the nucleic acid scaffold; (c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence; (d) determining an amplitude of Brownian noise of the nucleic acid scaffold in response to a force of less than 0.01 pN in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact with each other; (f) determining an amplitude of Brownian noise of the screening nucleic acid scaffold in response to the same force used in the absence of the first candidate molecule and the second candidate molecule by repeating (d); and (g) identify events of interaction between the two molecules, wherein the amplitude of Brownian noise is reduced compared to the reference amplitude at the same force in the absence of the molecules.

Also provided herein are methods of determining a binding interaction between between a first candidate molecule, a second candidate molecule and a third candidate molecule, the methods comprise: (a) providing a nucleic acid scaffold described herein, wherein the nucleic acid scaffold is positioned along an axis perpendicular to a bottom surface of the chamber of the device; (b) providing a device that comprises: (i) a chamber disposed within the device, wherein the chamber comprises a bottom surface, and (ii) a force application mechanism for stretching the contiguous polynucleotide sequence along the axis to form the nucleic acid scaffold; (c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence; (d) determining a reference amplitude of Brownian noise of the nucleic acid scaffold in response to a force of less than 0.01 pN in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact with each other; (f) contacting a third candidate molecule in solution to the screening nucleic acid scaffold; (g) determining an amplitude of Brownian noise of the screening nucleic acid scaffold in the presence of the third candidate molecule in response to the same force applied in (d); and (h) identify events of interaction between the first molecule, the second molecule and the third molecule, wherein the amplitude of Brownian noise is reduced compared to the reference amplitude at the same force in the absence of the first molecule, the second molecule and the third molecule.

Also provided herein are kits for determining a binding interaction between a first candidate molecule and a second candidate molecule. In some embodiments, the kits comprise: (a) a nucleic acid that comprises: (i) a contiguous polynucleotide sequence comprising: (I) a first end that comprises: (A) a first adapter for attaching to a bead, and (B) a first molecule binding sequence; (II) a second end that comprises: (A) a second adapter for attaching the nucleic acid to a bottom surface of a device, and (B) a second molecule binding sequence; (III) an intermediate portion between the first molecule binding sequence of the first end and the second molecule binding sequence of the second end, wherein the intermediate portion comprises: (A) a first pin forming sequence comprising a barcode, (B) a second pin forming sequence that is complementary to the first pin forming sequence, wherein the first pin forming sequence is hybridized to the second pin forming sequence, and (C) a loop linking the first pin forming sequence and the second pin forming sequence; (ii) a first spacer polynucleotide that has a polynucleotide sequence that is complementary to the first pin forming sequence, and (iii) a second spacer polynucleotide that has a polynucleotide sequence that is complementary to the second pin forming sequence, wherein the nucleic acid when attached as a nucleic acid scaffold to the bottom surface of the device requires a force of 10 pN to 30 pN to unfold the contiguous polynucleotide sequence (e.g., hairpin nucleic acid) along an axis perpendicular to the bottom surface of the device; and (b) a bead comprising an anchoring molecule configured to bind to the first adapter of the first end of the contiguous polynucleotide sequence of the nucleic acid. In some embodiments, each, the first molecule binding sequence and the second molecule binding sequence, independently comprises a small hairpin nucleic acid having a size in a range of from 5 to 100 bases. In some embodiments, the kits further comprise: (a) a first candidate molecule linked to a first glue sequence, wherein the first glue sequence is complementary to the first molecule binding sequence; and (b) a second candidate molecule linked to a second glue sequence, wherein the second glue sequence is complementary to the second molecule binding sequence. In some embodiments, the kits further comprise: (a) a first glue sequence comprising a first active group configured to be linked to a first candidate molecule, wherein the first glue sequence is complementary to the first molecule binding sequence; and (b) a second glue sequence comprising a second active group configured to be linked to a second candidate molecule, wherein the second glue sequence is complementary to the second molecule binding sequence. In some embodiments, the kits further comprise at least one of the first candidate molecule and the second candidate molecule. In some embodiments, the kits further comprise a third candidate molecule. In some embodiments, the first spacer polynucleotide and the second spacer polynucleotide are linked to each other by a spacer loop sequence, wherein the spacer loop sequence is hybridized to the loop.

### BRIEF DESCRIPTION OF THE DRAWINGS

Novel features of exemplary embodiments are set forth with particularity in the appended claims. A better understanding of the features and advantages will be obtained by reference to the following detailed description that sets forth illustrative embodiments, in which the principles of the disclosed systems and methods are utilized, and the accompanying drawings of which:

FIG. 1 depicts components of an exemplary hairpin nucleic acid according to an embodiment of the disclosure.

FIG. 2 provides non-limiting exemplary structures of two polynucleotide sequences that can be used for making a hairpin nucleic acid, the two polynucleotide sequence include: (a) a first polynucleotide sequence (SEQ ID NO: 1) encoding a first adapter 106, a first molecule binding sequence 104, a first junction sequence 112 and a first pin forming sequence 108 (from 5′ to 3′); and (b) a second polynucleotide sequence (SEQ ID NO: 2) encoding a second pin forming sequence 109, a second junction sequence 113, a second molecule binding sequence 105 and a second adapter 107 (from 5′ to 3′). The first pin forming sequence 108 and the second pin forming sequence 109 are complementary to each other, and hence form a double stranded region upon hybridization with each other. The first pin forming sequence 108 and the second pin forming sequence 109 can be linked to each other by a loop 110 to form the hairpin nucleic acid 100. The first junction sequence 112 and the second junction sequence 113 anchor a Y-shaped spacer polynucleotide to form a 4-way junction (e.g., Holiday junction). The two molecule binding sequences are neither identical nor complementary to each other. 5′-end of the first polynucleotide sequence is conjugated to a biotin, which can be used to anchor the hairpin nucleic acid to a bead. Similarly, the second adapter sequence can be used to immobilize the hairpin nucleic acid to a feature of a bottom surface of a device, wherein the feature is an oligonucleotide that is complementary to the nucleotide sequence of the second adapter 107. The letters K, X, Y and J represent any nucleotide (ACGT) natural or modified.

FIG. 3 shows an exemplary nucleotide sequence that is engineered to comprise a barcode. Briefly, as shown in FIG. 3, the nucleotide sequence was engineered to comprise multiple CCwGG (SEQ ID NO: 3) sequence motifs. When a plasmid containing the nucleotide sequence is propagated in E. coli, the DCM methylase present in E. coli methylates the second cytosine within the CCwGG (SEQ ID NO: 3) sequence, generating a specific epigenetic barcode that can be decoded (identified) on the device described here with an anti 5mC antibody. The nucleotide sequence also contained on either end a non-palindromic restriction site BsaI to ligate the junction sequences as well as the loop.

FIG. 4 shows exemplary 20 different barcodes that can be generated in silico by placing the constant sites at different positions within a fragment of 200 bases. An exemplary constant site comprised a nucleotide sequence of CCwGG (SEQ ID NO: 3). As shown, two constant sites spaced by 10 and 18 nucleotides respectively, were grafted to all the barcodes. The nucleotide sequence is methylated by the DCM methylase presents within E. coli and probed using an antibody against 5-mC. For aligning different nucleotides to the predicted sequences, the two constant sites present in all the nucleotides are used. The number of CCwGG (SEQ ID NO: 3) sequences within each nucleotide can be varied as well as the distance between two sites, allowing to generate a library a nucleotide fragments for multiplexing.

FIG. 5 depicts components of an exemplary spacer polynucleotide according to an embodiment of the disclosure.

FIG. 6 depicts non-limiting exemplary structures of two spacer polynucleotide strands that can be used for making a Y-shaped spacer polynucleotide, wherein the two spacer polynucleotide strands include: (a) a first spacer polynucleotide strand (SEQ ID NO: 5) encoding a polynucleotide sequence that is complementary to a contiguous portion of a first junction sequence binding region (505) and a first pin forming sequence binding region (503) (from 5′ to 3′); and (b) a second spacer polynucleotide strand (SEQ ID NO: 6) encoding a polynucleotide sequence that is complementary to a contiguous portion of a second pin forming sequence binding region (504) and a second junction sequence binding region (506) (from 5′ to 3′). These two polynucleotides can be annealed to form a Y-shape that can be ligated to the same barcode sequence used to construct the hairpin sequence. The first junction sequence and the second junction sequence anchor the first spacer polynucleotide strand and the second spacer polynucleotide strand, respectively, to form a 4-way junction (e.g., Holiday junction). The letters X′ and Y′ represent any nucleotide (ACGT) natural or modified.

FIG. 7 shows a non-limiting exemplary system comprising a holiday junction that is formed between a hairpin nucleic acid and spacer polynucleotides. As shown, the hairpin nucleic acid is attached to a bead (SEQ ID NO: 11) and a feature of a surface of a device. Upon application of sufficient force to the bead, the holiday junction resolves and causes strand invasion. After invasion, a first pin forming sequence and a second pin forming sequence of the hairpin nucleic acid hybridize to a first spacer polynucleotide strand and a second spacer polynucleotide strand, respectively, of the spacer polynucleotide. The arrows indicate where each component of the exemplary system are oriented and the overhang required for the cloning of the barcode sequence (SEQ ID NO: 4 as an example digested with the restriction enzyme BsaI).

FIG. 8 shows a graphical representation of method for identifying a hairpin nucleic acid 100 using a system described herein. As shown in A, at low force (below 12 pN), the hairpin sequence is fully hybridized, and the antibody can't bind to the methylated cytosine. As shown in B, upon increasing the force (above 20 pN), the hairpin molecule unfolds. As a result, the methylated cytosines (5mC) are now exposed, and the anti-5mC antibody has access to these modified bases to bind to them. The force is then reduced to or below 12 pN, causing the reformation of the hairpin. Accordingly, as shown in C, the fork is blocked transiently where there is an antibody bound. The position of this blockage is recorded. As shown in D, the first antibody bound is then ejected, and the fork continues its way toward the next antibody bound and the position of subsequence blockages are recorded. As this process is not destructive, the cycle can be repeated many times until all the positions are extracted.

FIG. 9 shows the alignment of 100 cycles of opening and closing of the hairpin in the presence of the antibody. Each star and dotted lines represent a position where the methylated cytosine has been detected. These positions can then be aligned to the predicted sequences to identify the barcode sequence.

FIG. 10 shows results of identifying nucleotides by decoding barcodes. Seven different plasmids, each containing a different barcode sequence, were mixed together at equimolar and digested with BsaI to generate a mixture of seven different nucleotide fragments. Then, the Y-shape nucleic acid and the loop were ligated at either end of the fragments and the resulting hairpin nucleic acid was attached to paramagnetic beads. The paramagnetic beads were injected in a flow cell and allowed to bind to the surface. After removing the non-bound beads, the antibody against 5-mC was injected in the flow cell and force cycles were performed to detect positions of all the methylated cytosine on all the nucleotides. After more than a hundred cycles, the blockage positions were extracted and mapped to their expected sequence by mapping the experimental blockages to their expected positions. HP=hairpin

FIG. 11 shows a graphical representation of method for making a nucleic acid scaffold from a hairpin nucleic acid 100 positioned between a bead and a feature of a surface of a device by performing strand invasion.

FIG. 12 shows a result of a successful invasion. Force was set constant at 5 pN while hairpin nucleic acid and the corresponding invading spacer polynucleotides are present within a flow cell. Starting at the arrow, the signature of the signal changed, which corresponds to the invasion of the intermediate portion 103 of a hairpin nucleic acid 100 by the spacer polynucleotide, indicating a successful strand invasion.

FIG. 13 shows a graphical representation of method for determining binding interactions between two candidate molecules using a system described herein.

FIG. 14 shows experimental traces of the interaction between two proteins (KU70/80 and APFL) on the double stranded substrate present on the scaffold DNA. Four different nucleotide sequences (SEQ ID NOS: 15-18) were used as the double stranded substrate. The interaction between the two proteins causes shortening of the Z displacement of the bead, as shown within the box. Once the interaction is broken, the scaffold fully stretches and reaches length similar to the length observed in the control experiment (in the buffer only, in the absence of the proteins)

FIGS. 15A-15B show exemplary traces for determining binding kinetics between the two candidate molecules. The force is maintained at 0.1 pN, which allows the scaffold to be enough flexible for the two proteins to interact (if they can interact). At low force, the bead attached to the scaffold moves due to Brownian motion with an amplitude that roughly correspond to the length of the scaffold. Upon interaction, the distance between the bead and the surface is reduced, which limit Brownian motion. Accordingly, binding kinetics is determined which corelates to how long the interaction lasts by tracking the movement of the bead over time,

FIG. 16 shows exemplary changes in Brownian movement of a bead anchoring a nucleic acid scaffold in the presence and absence of a force relative to a surface of a device. The human receptor ACE2 and SARS-COV2 RBD domain were both tagged with oligonucleotides that allow to anchor them to molecule binding sequence 104 and 105 respectively. When the force was reduced to <0.1 pN, the proteins showed the ability to interact. The interaction is characterized by a reduction in the amplitude of the Brownian noise (indicated by the arrow in FIG. 16). Upon the increase in force (approximatively 2 pN), the interaction is broken. Upon reduction to low force (<0.1 pN), the interaction is observed again as this is a non-destructive process. (indicated by the arrows in FIG. 16).

FIG. 17 shows the universal barcode sequence that can serve as the basis for all the subsequent barcodes. This sequence can also be used for generating the universal strand invasion hairpin. Briefly, as shown in FIG. 17, the nucleotide sequence (SEQ ID NO: 35) was engineered to comprise at the 5′ and 3′ extremities two CCwGG (SEQ ID NO: 3) sequence motifs separated by 20 and 10 bases respectively that will always be included in all barcodes and therefore always be methylated in all barcode fragment generated. Then, fourteen CawGG (SEQ ID NO: 54) sites were inserted between these reference positions at different spacing between each site. The sequence has also been generated to be uniform in the GC content throughout the fragment (global GC content is 43%). When a new barcode needs to be generated, any of these positions (between 1 to 14 positions can be mutated and therefore give a theorical maximum number of barcode to 214 fragments) can be converted to CCwGG, which will be methylated by the E. coli DCM methylase when the plasmid containing this nucleotide sequence is propagated in bacteria (SEQ ID NO: 3). When this fragment will be isolated from the plasmid, it will generate a specific epigenetic barcode that can be decoded on the platform with an anti-5mC antibody. The nucleotide sequence also contained on either end a non-palindromic restriction site BsaI to generate the 4-bases overhang that are required for the ligation of the junction sequences as well as the loop.

FIG. 18 shows examples of five different patterns of methylation as well as the universal strand invasion fragment that were generated from the sequence presented in FIG. 3 by changing four or five CawGG sequence into CCwGG (SEQ ID NO: 39-44). As shown, two constant sites spaced by 10 and 20 nucleotides respectively, were grafted to all the barcodes as reference at the 3′ and 5′ end respectively. The nucleotide sequence is methylated by the DCM methylase presents within E. coli and probe using the antibody against 5-mC. For aligning different nucleotides to their respective predicted sequences, the four constant sites (two at both end) present in all the barcodes are used. The number of CCwGG (SEQ ID NO: 3) sequences within each fragment can be varied allowing to generate a library a nucleotide fragments for multiplexing with a maximal number of sequences being 214.

FIG. 19A provides a graphical representation of components of a nucleic acid scaffold from a hairpin nucleic acid 100 positioned between a bead and a feature of a surface of a device, wherein each, the first molecule binding sequence 104 and second molecule binding sequence 105, are small hairpin nucleic acids. FIG. 19B shows a graphical representation of small hairpin nucleic acid, which allows to ensure that a protein/molecule is anchored to the scaffold. As shown in FIG. 19B, the small hairpin nucleic acid 104 comprises a first molecule binding glue complementary strand 601 and a second molecule binding glue complementary strand 602 that are linked to each other by molecule binding complementary loop 605. The first molecule binding glue complementary strand 601 comprises a a first molecule binding pin forming sequence binding region 603. The second molecule binding glue complementary strand 602 comprises a second molecule binding pin forming sequence binding region 604. The GC content of the pin sequence 602 and its complementary sequence 603 is more than 90% to ensure that the opening of this sequence doesn't interfere with the opening of the barcode sequence. The structure of the sequence 105 is similar to the structure presented in FIG. 19B, except that the sequence 601 and 602 will be different from sequence 104 to allow anchoring specific target molecule. FIG. 19C shows an exemplary glue polynucleotide covalently linked to a candidate molecule. As shown in FIG. 19C, the glue polynucleotide 700 comprises a first glue spacer polynucleotide strand 701 and a second glue spacer polynucleotide strand 702 that are linked to each other by glue loop 707. The first glue spacer polynucleotide strand 701 comprises a first glue junction sequence binding region 705 and a first glue pin forming sequence binding region 703. The second glue spacer polynucleotide strand 702 comprises a second glue junction sequence binding region 706, a second glue pin forming sequence binding region 704 and a single stranded sequence 708 between the hairpin and the candidate molecule. The second glue junction sequence binding region 706 can be covalently attached to a candidate molecule.

FIG. 20A shows expected traces of binding of glue sequences that are linked to candidate molecules. FIG. 20B shows a prototype sequence of the sequence 104 and 105 presented in FIG. 1. These sequences are composed of two variable sequences of 12 bases (part 1 and 3) on either side of the small hairpin sequence (part 2) to allow specific protein to be anchored on specific scaffold structure. FIG. 20C shows a prototype sequence of the oligonuccleotide covalently attached to the target molecule. The sequence 3 and 5 correspond to the complementary sequence present on the scaffold structure (Sequence of part 1 and 3 in FIG. 20B). These sequences will allow the anchoring of specific target molecule on distinct structure. The sequence 1 (with the associated TTT of sequence 2) can be located on either the 5′ or 3′ end of the invading oligonucleotide. The sequence and its length can be between 10 to 50 nucleotides to allow flexibility of the system. FIG. 20D shows examples of how the anchoring of the target molecules can be monitored. Two small closing jumps of about 30 nm are observed (indicated by the arrows) as well as the closing jump due to the opening of the hairpin. After addition of the first invading oligonucleotide (for this proof-of-concept, there was no target molecule attached to the oligonucleotide), one of the two jumps disappeared and only one jump is observed. Upon addition of the second oligonucleotide, the second closing jump disappear as well due to the invasion of the second small hairpin. The letters X and Y represent any nucleotide (ACGT) natural or modified.

FIG. 21 shows results of identifying the different fragments by decoding barcodes. The six fragments described in FIG. 18 were ordered, cloned into plasmid and propagated into E. coli such that all CCwGG sites within these sequences are methylated. After digestion of the plasmids with BsaI in parallel, each containing a different barcode sequence, the Y-shape nucleic acid and the loop were ligated at both end of these fragments. The six resulting nucleic acid hairpins were attached to paramagnetic beads and mixed at an equimolar ratio. The paramagnetic beads were injected in a flow cell and allowed to bind to the surface. After removing the non-bound beads, the antibody against 5-mC was injected in the flow cell and force cycles were performed to detect positions of all the methylated cytosine on all the hairpins. After more than a hundred cycles, the blockage positions were extracted and mapped to their expected sequence by mapping the experimental blockages to their expected positions. BC=barcode

FIG. 22 provides a graphical representation of method for making a nucleic acid scaffold from a hairpin nucleic acid 100 positioned between a bead and a feature of a surface of a device by performing strand invasion. Briefly, the universal strand invasion hairpin (SEQ ID. NO: 35) is injected into the flow cell and through complementary between the first and second junction sequence, bind to the hairpin.

FIG. 23 shows an exemplary trace for the events described in FIGS. 22A-22C. Briefly, as shown in FIG. 23, upon increasing the force between 5 to 10 pN, the holiday junction formed is resolved and the invasion can be observed by an extension of the molecule of approximately 200 nm.

FIG. 24 shows efficiency of strand invasion in a hairpin nucleic acid comprising universal barcode sequences. Briefly, five hairpin nucleic acids (SEQ ID NOS: 40-44) were generated and analyzed for strand invasion efficiency.

FIG. 25A shows a graphical representation of how a molecular interaction is probed to determining binding interactions between two candidate molecules using a system described herein. Briefly, after the attachment of both target molecules on the scaffold structure, the force is reduced at 0.01 pN in order for the two molecules to interact. The force is then increased to a value between 0.5 to 20 pN, depending on the strength of the interaction. If the two molecules were interacting prior to this increase in force, the full extension of the dsDNA will be prevented, and an intermediate position will be observed. After the interaction is broken, the dsDNA scaffold will fully extend and cause a sudden jump in the position of the bead. The time and the force required to break this interaction can then be used to extract the energetic of the interaction (entropy, enthalpy and ΔG). FIG. 25B shows traces of the formation of the complex composed of KU70/80 (which binds at the double stranded breaks) and the protein APFL. Briefly, two adapters, each composed of two oligonucleotides forming a double stranded region as well as a single stranded glue sequence complementary to the first and second adapter sequence located on either side of the scaffold, were injected into the flow cell and the position of the beads were recorded over 100 force cycles (only 14 cycles are shown in this figure). In the absence of any proteins, the scaffold gets fully elongated when the force is increased to 2 pN (top panel). Upon injection of the Ku70/80 complex as well as the protein APFL, the complex of Ku70/80 form at the blunt end of both adapters and the protein APFL bridges these complexes. This can be observed by the transient shorter extension of the scaffold when the complex is formed (indicated by the box, bottom panel)

FIG. 26A shows a graphical representation of how binding kinetics are determined between the two candidate molecules at constant low force. The force is maintained at 0.01 pN, which allows the scaffold to be enough flexible for the two proteins to interact (if they can interact). At this low force, the bead attached to the scaffold moves due to Brownian motion with an amplitude that corresponds to the length of the scaffold (0.3 nm per base pair and the scaffold is composed of 900 bp or 250 nm displacement). Upon interaction, the distance between the bead and the surface is reduced, which limit Brownian motion (the scaffold is reduced to only 300 bpm which represent only 100 nm of displacement). The binding kinetics can be determined, which correspond to how many events are observed over the duration of the non-interacting state (Kon) and the Koff corresponds to the average of all the interactions detected. FIG. 26B shows a real trace of the interaction between the human receptor protein ACE2 and the wildtype SARS-COV2 viral receptor binding protein (RBD). The five interactions between these two proteins detected within this 5-minute recording are indicated by a red line. FIG. 26C shows two phases of each 5 minutes separated by a phase at 20 pN (to reset all the molecules to the non-interacting state). As the process is non-destructive, it is possible to observe interactions after this “reset” phase.

FIG. 27 shows an example of the formation of a tertiary complex between the E3 ligase CEREBLON (in complex with its binding partner DDB1), the neo-target GSPT1 and the molecular glue CC885. In the absence of the molecular glue, short interacting events can be observed (not visible on this figure) but the addition of the molecular glue greatly stabilizes the complex. In fact, the complex can only be broken when the force is increased to 20 pN and the complex is reformed with few second when the force is reduced to 0.01 pN.

FIG. 28 shows an example of formation of a tertiary complex between the E3 ligase CEREBLON (in complex with its binding partner DDB1), the neo-target GSPT1 and the molecular glue thalidomide. In the presence of the molecular glue, it is possible to observe formation of the tertiary complex. As opposed to the molecular glue CC885, the interaction is weaker and can be broken even at low force.

FIG. 29 shows formation of a tertiary complex between the E3 ligase CEREBLON (in complex with its binding partner DDB1), the neo-substrate IKAROS1 as well as the molecular glue pomalidomide. In the absence of the small molecules, there is either no interaction or the interactions are really short (indicated by the small triangles). Upon addition of the molecular glue pomalidomide, strong tertiary complex is formed (indicated by the arrow). This complex is so strong that it can sustain up to 2 pN of force (indicated by the star).

FIG. 30 demonstrates multiplexing capabilities of systems described herein. Two different barcodes were constructed, each with the same complementary sequence, to anchor the ACE2 protein and the other end with two different single stranded sequences: one to glue RBD wild type protein and the second sequence specific for the RBD delta mutant sequence. Once randomly attached to the flow cell, the barcodes were “read” to determine the coordinates of each barcode within the flow cell. Then, the universal strand invasion hairpin was injected into the flow cell and the force set at 5pN to allow the conversion of the hairpin into a dsDNA scaffold. The ACE2 and RBD wild type was first injected into the flow cell and then, constant force cycles were performed. Interactions were observed only on the molecules specific for the RBD wild type. No interaction was observed on the scaffolds that were specific for RBD delta mutant. The RBD delta mutant was then injected into the flow cell and the low force cycles were repeated. Interaction on both scaffold (for the wild type and mutant RBD) were observed simultaneously in the same flow cell, demonstrating that it is possible to measure binding kinetic of different interaction at the same time within the same flow cell.

### DETAILED DESCRIPTION

### Overview

This disclosure is directed to the analysis of molecular interactions between two or more molecules in a highly parallel way at the single molecular level using the magnetic tweezers. There is a high demand to understand the interaction between different proteins or protein and small molecule but they most of the time rely on indirect methods, like co-immunoprecipitation or co-localization.

Disclosed herein are methods of determining binding interactions between two or more candidate molecules in real-time and at the single molecule level. In some embodiments, the methods comprise anchoring a candidate molecule (a protein or a nucleic acid) to a bead. In some embodiments, the methods comprise generating a force sufficient to pull on the candidate molecule and stretch it. Depending on the structure of the candidate molecule, different signals are expected. For example, in the case of a hairpin, reaching >20 pN of force allows to mechanically denature the nucleic acid and obtain two distinct and complementary single stranded strands. Accordingly, in some embodiments, the methods are used for analyzing physical proprieties of the candidate molecule (e.g., DNA, RNA or a combination thereof). Alternatively, in some embodiments, the methods are used for determining location(s) of interaction of various enzymes (e.g., polymerases, helicases, topoisomerases) with the candidate molecule. Moreover, in some embodiments, the methods are used for analyzing binding kinetics of one candidate molecule (e.g., oligonucleotides or proteins (for example antibodies)) toward another candidate molecule. However, most molecular interactions between proteins or small molecules and proteins are relatively weak (≤5 pN). Such molecular interactions are difficult to analyze on the magnetic tweezer (the resolution is bad at low forces as the Brownian movement is high). Specifically, a weak force of interaction may result in too high noise to be able to extract the proper signal (signal to noise, S/N, is too low). Methods and systems disclosed herein are advantageously capable of: analyzing the molecular interactions at the single molecule level; measuring multiple interactions in parallel; determining locations of the interaction by directing positions of the candidate molecules (either protein or small molecules) in solution coupled to the system; and amplifying signal (interaction or not) well above the noise to be able to determine molecular interactions wherein the force of interaction is ≤0.1pN.

### Systems

Provided herein are systems comprising nucleic acid scaffolds. In some embodiments, nucleic acid scaffolds comprise hairpin nucleic acids. In some embodiments, the hairpin nucleic acids comprise two molecule binding sequences and two spacer binding sequences. In some embodiments, the two spacer binding sequences are linked by a loop and form a contiguous polynucleotide sequence. In some embodiments, each of the two spacer binding sequences comprises a pin forming sequence and, optionally, a junction sequence. In some embodiments, the nucleic acid scaffolds further comprise one or two spacer polynucleotides. In some embodiments, the nucleic acid scaffolds further comprise two candidate molecules each linked to a candidate molecule. In some embodiments, the nucleic acid scaffolds are anchored to beads. In some embodiments, a nucleic acid scaffold is anchored to a bead by a non-covalent interaction, wherein the first end comprises a first adapter, wherein the bead comprises a first handle sequence conjugated to the bead, wherein the first adapter and the first handle sequence are complementary to each other. In some embodiments, the system further comprises a device (e.g., chamber) comprising one or more features. In some embodiments, a feature of a device described herein is configured to immobilize a nucleic acid scaffold described herein. In some embodiments, a nucleic acid scaffold is immobilized to a feature of a device by a non-covalent interaction, wherein the second end comprises a second adapter, wherein the feature comprises a second handle sequence conjugated to the feature, wherein the second adapter and the second handle sequence are complementary to each other. In some embodiments, the device is capable of exerting a force to the bead, thereby causing the bead to move. In some embodiments, the feature is operatively linked to a sensor that is capable of measuring movement of the bead. In some embodiments, the movement of the bead is up and down.

### Hairpin Nucleic Acid

Disclosed herein is a hairpin nucleic acid that is capable of being positioned between a bead and a feature of a surface of a device. In some embodiments, the hairpin nucleic acid is a contiguous polynucleotide sequence. In some embodiments, the hairpin nucleic acid has a Y shape to allow anchoring on the bead on one side and to the surface at the other end. In some embodiments, the hairpin nucleic acid comprises two molecule binding sequences, two spacer binding sequences and two adapters. In some embodiments, the spacer binding sequence comprises two pin forming sequences, a loop and two junction sequences. In some embodiments, the pin forming sequence comprises one or more barcodes. In some embodiments, the hairpin nucleic acid comprises a polynucleotide having a size between 10 bases and 1500 bases. In some embodiments, the hairpin nucleic acid comprises a polynucleotide having a size between 15 bases and 2000 bases. In some embodiments, the hairpin nucleic acid comprises a polynucleotide having a size between 50 bases and 1000 bases.

In some embodiments, the hairpin nucleic acid molecule is chemically synthesized. In some embodiments, the hairpin nucleic acid molecule is produced via in vitro transcription. In some embodiments, the hairpin nucleic acid molecule is produced via a polymerase chain reaction. In some embodiments, the hairpin nucleic acid molecule(s) is (are) purified from a cell. In some embodiments, the hairpin nucleic acid molecule is chemically synthesized, produced via in vivo transcription, produced via a polymerase chain reaction, purified from a cell, or a combination thereof.

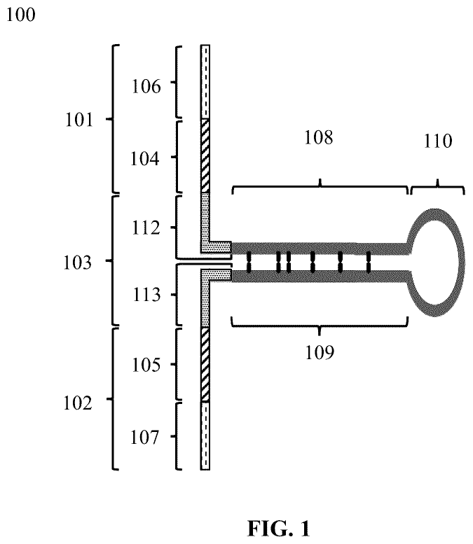

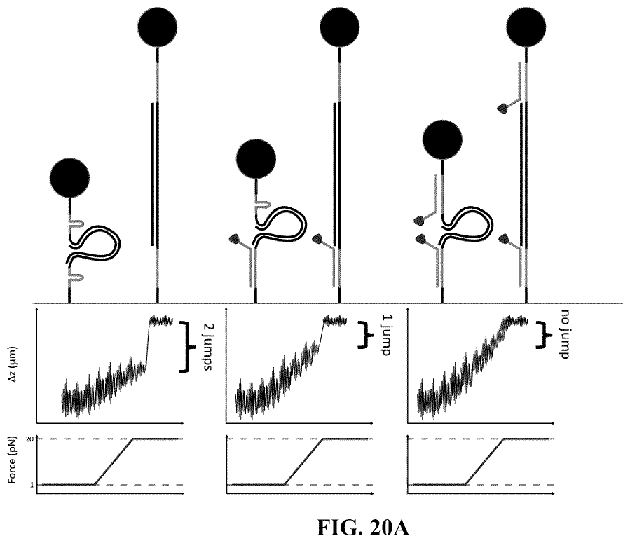

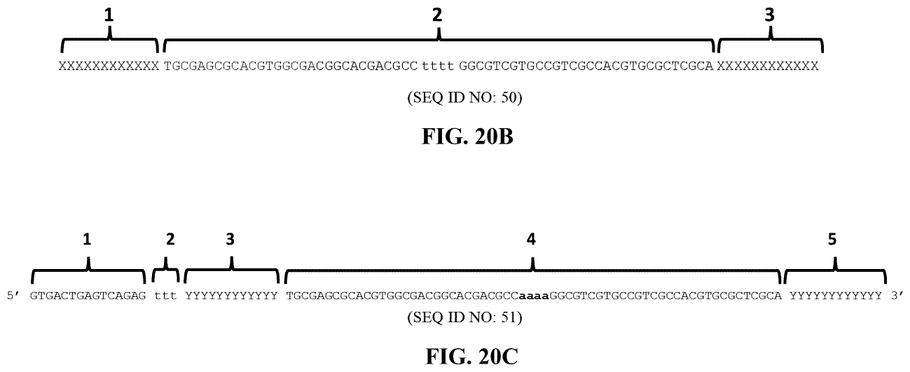

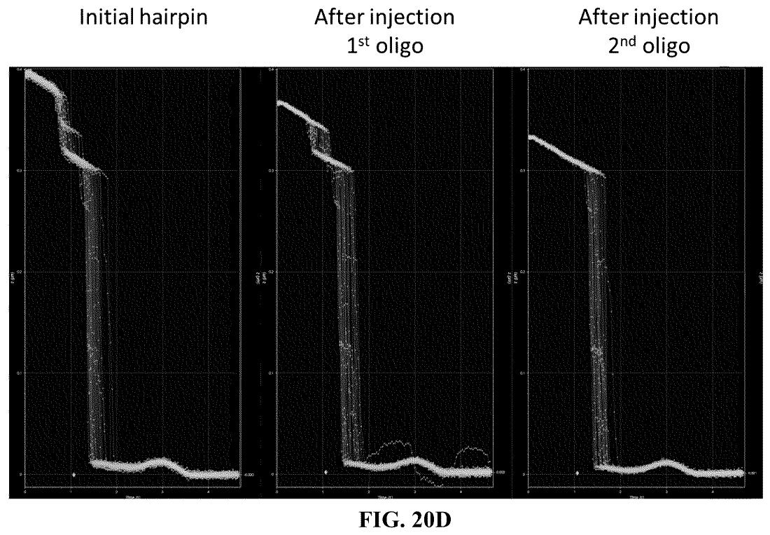

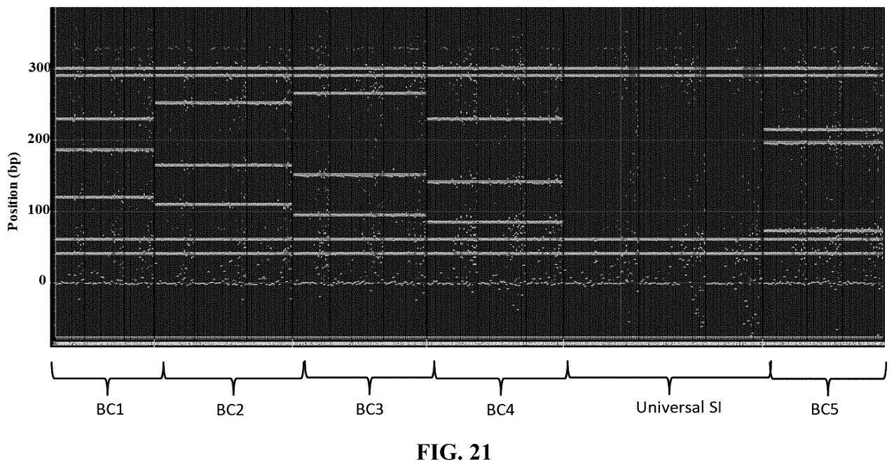

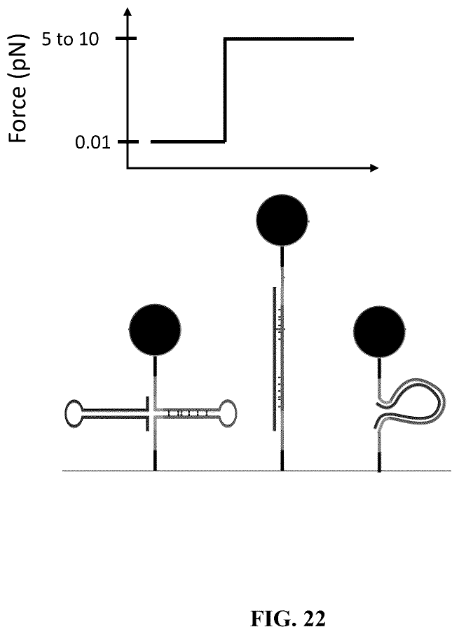

FIG. 1 shows an exemplary structure of a hairpin nucleic acid 100. As shown, the hairpin nucleic acid 100 comprises a contiguous polynucleotide sequence comprising a first end 101, a second end 102 and an intermediate portion 103 between the first end 101 and the second end 102. As shown, the first end 101 comprises a first molecule binding sequence 104 and the second end 102 comprises a second molecule binding sequence 105. In some embodiments, the first molecule binding sequence 104 hybridizes to a first glue sequence of the first candidate molecule. In some embodiments, the second molecule binding sequence 105 hybridizes to a second glue sequence of a second candidate molecule. In some embodiments, the first end 101 is anchored to a bead. In some embodiments, the first end 101 is anchored to the bead by a covalent interaction or a noncovalent interaction (e.g., streptavidin-biotin interaction). Alternatively, in some embodiments, the first end 101 comprises a first adapter 106 located at terminal end of the first end 101. In some embodiments, the first adapter 106 is capable of hybridizing to a first handle sequence conjugated to the bead, wherein the first adapter 106 and the first handle sequence are complementary to each other. In some embodiments, the second end 102 is immobilized to a feature of a surface of a device. In some embodiments, the second end 102 is immobilized to the feature by a covalent interaction or a noncovalent interaction (e.g., strep tag-biotin interaction). Alternatively, in some embodiments, the second end 102 comprises a second adapter 107 located at terminal end of the second end 102. In some embodiments, the second adapter 107 is capable of hybridizing to a second handle sequence conjugated to the feature, wherein the second adapter 107 and the second handle sequence are complementary to each other. As shown, the intermediate portion 103 is located between the first molecule binding sequence 104 and the second molecule binding sequence 105. In some embodiments, the intermediate portion 103 comprises a hairpin structure. In some embodiments, the intermediate portion 103 comprises a contiguous nucleotide sequence comprising a first spacer binding sequence (not shown) and a second spacer binding sequence (not shown), wherein the first spacer binding sequence is located between the first molecule binding sequence 104 and the second spacer binding sequence. Accordingly, in some embodiments, the second spacer binding sequence is located between the first spacer binding sequence and the second molecule binding sequence 105. In some embodiments, the first spacer binding sequence comprises a first pin forming sequence 108 and the second spacer binding sequence comprises a second pin forming sequence 109. Accordingly, in some embodiments, the intermediate portion comprises a contiguous nucleotide sequence comprising the first pin forming sequence 108 and the second pin forming sequence 109. In some embodiments, a nucleotide sequence of the first pin forming sequence 108 and a nucleotide sequence of the second pin forming sequence 109 are complementary to each other. In some embodiments, the first spacer binding sequence and the second spacer binding sequence are linked by a loop 110 to form a contiguous polynucleotide sequence. Accordingly, in some embodiments, the loop 110 is located between the first pin forming sequence 108 and the second pin forming sequence 109. In some embodiments, the first pin forming sequence 108, the loop 110 and the second pin forming sequence 109 forms a hairpin structure. As shown in FIG. 1, in some embodiments, the first spacer binding sequence further comprises a first junction sequence 112 that is located between the first molecule binding sequence 104 and the first pin forming sequence 108. Similarly, as shown in FIG. 1, in some embodiments, the second spacer binding sequence further comprises a second junction sequence 113 that is located between the second molecule binding sequence 105 and the second pin forming sequence 109. The first junction sequence 112 and the second junction sequence 113 may not be complementary to each other. FIG. 20B shows a prototype sequence of the sequence 104 and 105 presented in FIG. 1.

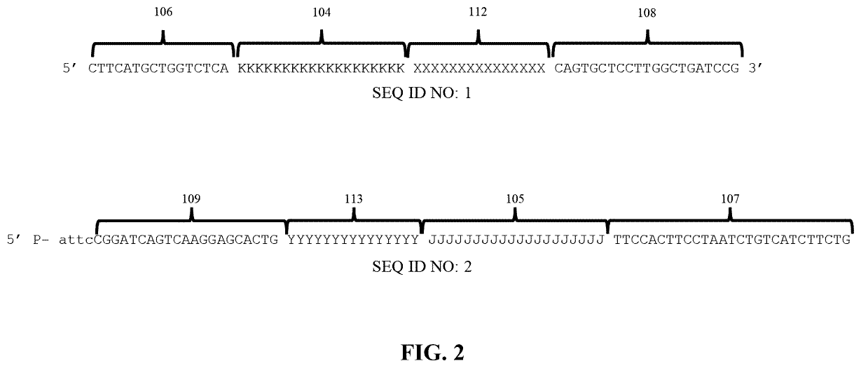

FIG. 2 provides non-limiting exemplary structures of two polynucleotide sequences that can be used for making a Y-shape nucleic acid that can further be used for making a hairpin nucleic acid, the two polynucleotide sequence include: (a) a first polynucleotide sequence (SEQ ID NO: 1) encoding a first adapter (106), a first molecule binding sequence (104), a first junction sequence (112) and a first pin forming sequence (108) (from 5′ to 3′); and (b) a second polynucleotide sequence (SEQ ID NO: 2) encoding a second pin forming sequence (109), a second junction sequence (113), a second molecule binding sequence (105) and a second adapter (107) (from 5′ to 3′). An exemplary nucleotide sequence encoding a second pin forming sequence (109), a second junction sequence (113), a second molecule binding sequence (105) and a second adapter (107) (from 5′ to 3′) is provided in SEQ ID NO: 33.

### Molecule Binding Sequence(s)

In some embodiments, a first end 101 and a second end 102 of a hairpin nucleic acid 100 comprise a first molecule binding sequence 104 and a second molecule binding sequence 105, respectively. In some embodiments, the first molecule binding sequence 104 and the second molecule binding sequence 105 are non-identical to each other. In some embodiments, the first molecule binding sequence 104 and the second molecule binding sequence 105 are non-complementary to each other. In some embodiments, the first molecule binding sequence 104 and the second molecule binding sequence 105 comprise an intermediate portion 103 of the hairpin nucleic acid between the first molecule binding sequence 104 and the second molecule binding sequence 105. In some embodiments, the first molecule binding sequence 104 and the second molecule binding sequence 105 independently have a size in a range of from 5 bases to 100 bases, from 5 bases to 80 bases, from 5 bases to 60 bases, from 5 bases to 40 bases, from 5 bases to 20 bases, from 10 bases to 100 bases, from 10 bases to 80 bases, from 10 bases to 60 bases, from 10 bases to 40 bases, from 10 bases to 20 bases, from 20 bases to 100 bases, from 20 bases to 80 bases, from 20 bases to 60 bases, from 20 bases to 40 bases, from 40 bases to 100 bases, from 40 bases to 80 bases, from 40 bases to 60 bases, from 60 bases to 100 bases, from 60 bases to 80 bases, or from 80 bases to 100 bases.

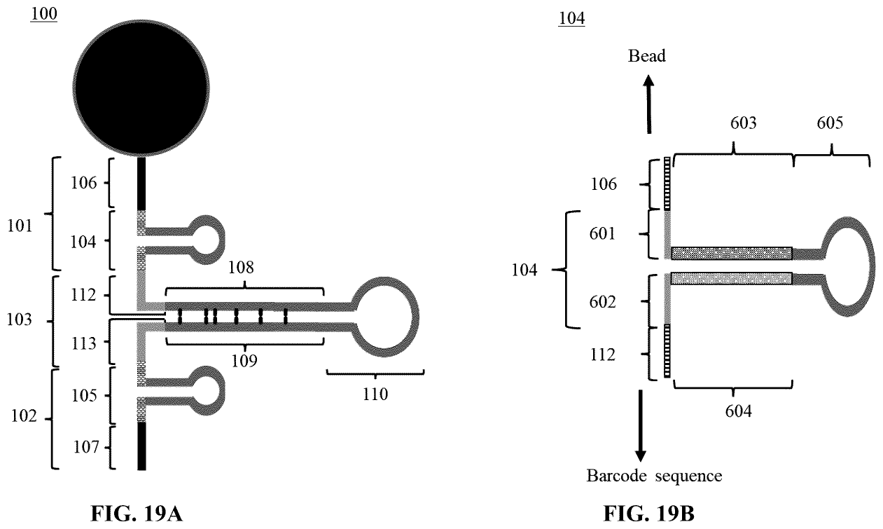

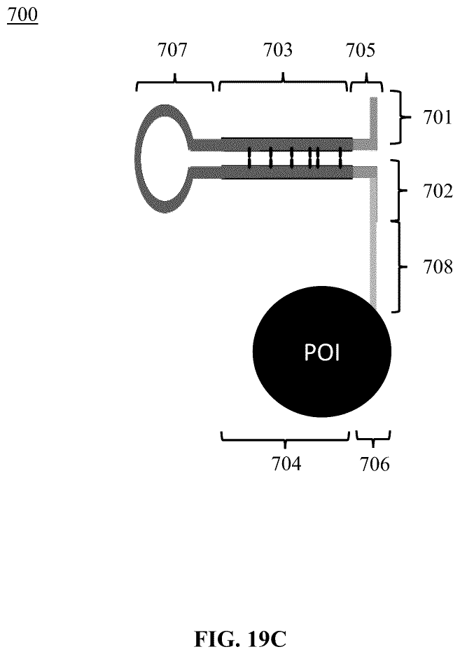

In some embodiments, a molecule binding sequence comprises a small hairpin nucleic acid. In some embodiments, a small hairpin nucleic acid has a size in a range of from 5 bases to 100 bases, from 5 bases to 80 bases, from 5 bases to 60 bases, from 5 bases to 40 bases, from 5 bases to 20 bases, from 10 bases to 100 bases, from 10 bases to 80 bases, from 10 bases to 60 bases, from 10 bases to 40 bases, from 10 bases to 20 bases, from 20 bases to 100 bases, from 20 bases to 80 bases, from 20 bases to 60 bases, from 20 bases to 40 bases, from 40 bases to 100 bases, from 40 bases to 80 bases, from 40 bases to 60 bases, from 60 bases to 100 bases, from 60 bases to 80 bases, or from 80 bases to 100 bases. An exemplary hairpin nucleic acid comprising small hairpin nucleic acid(s) is shown in FIG. 19A. FIG. 19B shows graphical representation of a molecule binding sequence that comprises a small hairpin nucleic acid. In some embodiments, a small hairpin nucleic acid allows to ensure that a protein/molecule is anchored to a nucleic acid scaffold described herein.

### Spacer Binding Sequences

In some embodiments, an intermediate portion 103 of a hairpin nucleic acid 100 comprises two spacer binding sequences. In some embodiments, the two spacer binding sequences form a contiguous polynucleotide sequence. In some embodiments, the two spacer binding sequences are located between two molecule binding sequences of the hairpin nucleic acid 100. In some embodiments, the two spacer binding sequences comprise a first spacer binding sequence and a second spacer binding sequence. In some embodiments, at least a portion of the first spacer binding sequence is complementary to at least a portion of the second spacer binding sequence. In some embodiments, the first spacer binding sequence and the second spacer binding sequence upon linked to each other form a contiguous polynucleotide sequence, wherein the first spacer binding sequence and the second spacer binding sequence upon hybridization with each other can form a hairpin structure. In other words, in some embodiments, the first spacer binding sequence and the second spacer binding sequence, upon hybridize to each other and their ligation to the pin sequence 108 and 109 as well as the loop sequence 110 allows the formation a contiguous polynucleotide sequence, wherein the first spacer binding sequence and the second spacer binding sequence form a hairpin structure. In some embodiments, each of the two spacer binding sequences comprises a pin forming sequence. Accordingly, in some embodiments, the two pin forming sequences upon linked to each other by a loop forms a hairpin structure. In some embodiments, the pin forming sequence comprises one or more barcodes.

### Pin Forming Sequence(s)

In some embodiments, a first spacer binding sequence and a second spacer binding sequence of a hairpin nucleic acid 100 comprise a first pin forming sequence 108 and a second pin forming sequence 109, respectively. Accordingly, in some embodiments, the first pin forming sequence 108 and the second pin forming sequence 109 are located between two molecule binding sequences of the hairpin nucleic acid 100. In some embodiments, the first pin forming sequence 108 and the second pin forming sequence 109 are complementary to each other. In some embodiments, the first pin forming sequence 108 and the second pin forming sequence 109 hybridize to each other. In some embodiments, a size of each of the two pin forming sequences is in a range of from 5 bases to 500 bases, from 5 bases to 400 bases, from 5 bases to 300 bases, from 5 bases to 200 bases, from 5 bases to 100 bases, from 5 bases to 80 bases, from 5 bases to 60 bases, from 5 bases to 40 bases, from 5 bases to 20 bases, from 10 bases to 500 bases, from 10 bases to 400 bases, from 10 bases to 300 bases, from 10 bases to 200 bases, from 10 bases to 100 bases, from 10 bases to 80 bases, from 10 bases to 60 bases, from 10 bases to 40 bases, from 10 bases to 20 bases, from 20 bases to 500 bases, from 20 bases to 400 bases, from 20 bases to 300 bases, from 20 bases to 200 bases, from 20 bases to 100 bases, from 20 bases to 80 bases, from 20 bases to 60 bases, from 20 bases to 40 bases, from 40 bases to 500 bases, from 40 bases to 400 bases, from 40 bases to 300 bases, from 40 bases to 200 bases, from 40 bases to 100 bases, from 40 bases to 80 bases, from 40 bases to 60 bases, from 60 bases to 500 bases, from 60 bases to 400 bases, from 60 bases to 300 bases, from 60 bases to 200 bases, from 60 bases to 100 bases, from 60 bases to 80 bases, from 80 bases to 500 bases, from 80 bases to 400 bases, from 80 bases to 300 bases, from 80 bases to 200 bases, or from 80 bases to 100 bases.

### Barcode

In some embodiments, pin forming sequences described herein comprise one or more barcodes. In some embodiments, decoding of the one or more barcodes reveals an underlying nucleotide sequence. For example, in some embodiments, the epigenetic modification pattern of the hairpin nucleic acid is used for decoding the barcode. In some embodiments, the epigenetic modification pattern is generated using a protein (e.g., monoclonal antibody) that binds to an epigenetically modified nucleotide. Non-limiting examples of the epigenetic modifications of DNA include 3-methylcytosine (3mC) modification, 4-methylcytosine (4mC) modification, 5-methylcytosine (5mC) modification, 5-hydroxymethylcytosine (5hmC) modification, 5-formylcytosine (5fC) modification, 5-carboxylcytosine (5caC) modification, 6-methyladenosine (m6A) modification or a combination thereof. Non-limiting examples of the epigenetic modifications of RNA include 5-hydroxymethyluracil (5hmU) modification, pseudo-uridine modification or a combination thereof. Non-limiting examples of the epigenetic modifications of DNA or RNA include 3-methyl cytosine (3mC) modification, N6-methyladenosine (m6A) modification or a combination thereof. In some embodiments, the epigenetic modification of DNA comprises 5-methylated cytosine modification(s).

Accordingly, in some embodiments, the epigenetic modified nucleotide comprises one or more methylated cytosines. In other embodiments, an enzyme introducing an epigenetic modification pattern is used. In some embodiments, the epigenetic modified nucleotide comprises one or more methylated adenosines.

In some embodiments, the barcode comprises one or more constant sites. In some embodiments, the barcode comprises at least one, at least two, at least three, at least four, at least five, at least six, at least seven, at least eight, at least nine, at least ten, at least eleven, at least twelve, at least thirteen, at least fourteen, or more constant sites. In some embodiments, the barcode comprises one, two, three, four, five, six, seven, eight, nine, ten, eleven, twelve, thirteen, fourteen, or more constant sites. In some embodiments, the barcode comprises one to fourteen, three to fourteen, seven to fourteen, ten to fourteen, two to twelve, five to twelve, seven to twelve, ten to twelve, one to ten, three to ten, seven to ten, three to eight, five to eight, or two to seven constant sites. In some embodiments, the barcode comprises two constant sites. In some embodiments, the one or more constant sites are independently present within first 8-20 nucleotides from 5′-end of the pin forming sequence, within first 8-20 nucleotides from 3′-end of the pin forming sequence or both. In some embodiments, at least two constant sites are identical among all the barcode fragments. In some embodiments, at least two constant sites are different relative to each other. In some embodiments, at least two constant sites are spaced 5 to 50, 10 to 50, 20 to 50, 30 to 50, 40 to 50, 5 to 40, 10 to 40, 20 to 40, 30 to 40, 5 to 30, 10 to 30, 20 to 30, 5 to 20, 10 to 20 or 5 to 10 bases apart. In some embodiments, the barcode comprises two constant sites. In some embodiments, one or more constant sites comprise a nucleotide sequence that undergoes epigenetic modification. In some embodiments, one or more constant sites comprise one or more cytosines, one or more adenines, or combinations thereof. In some embodiments, one or more cytosines are methylated by the DCM methylase of E. coli. In some embodiments, one or more adenosines are methylated by the DAM methylase of E. coli. In some embodiments, one or more nucleotides of SEQ ID NO: 3 in the barcode sequence are methylated by a methylase. In some embodiments, all second C of CCwGG (SEQ ID NO: 3) are methylated. In some embodiments, at least second C of CCwGG (SEQ ID NO: 3) from 5′-terminal is methylated. In some embodiments, second C from 5′-terminal of CCwGG (SEQ ID NO: 3) is methylated.

In some embodiments, a constant site comprises a nucleotide sequence of CCwGG (SEQ ID NO: 3). In some embodiments, when a plasmid encoding a constant site having a nucleotide sequence of CCwGG (SEQ ID NO: 3) is propagated in E. coli, the DCM methylase present in E. coli methylates the second cytosine within the CCwGG (SEQ ID NO: 3) sequence.

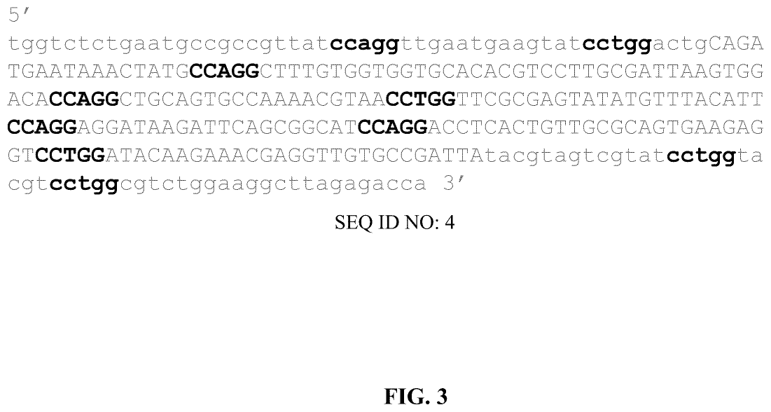

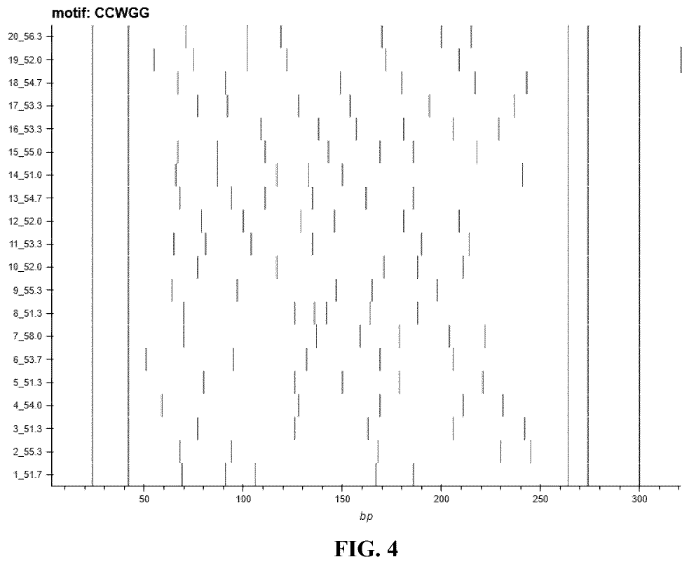

FIG. 3 shows an exemplary nucleotide sequence of the first hairpin nucleic acid precursor. FIG. 4 shows 20 exemplary barcodes that can be generated in silico by placing two constant site sequences within a nucleotide at constant positions at either end of the fragment plus the central region which contains various number of CCwGG sequences (SEQ ID NO: 3) spaced by different number of bases. Accordingly, in some embodiments, exemplary barcodes can be generated in silico by placing two constant site sequences within a nucleotide at different positions. In some embodiments, the constant site comprises a nucleotide sequence of CCwGG (SEQ ID NO: 3). In some embodiments, the constant site comprises a methylated nucleotide sequence of CCwGG (SEQ ID NO: 3).

In some embodiments, a barcode is generated from a universal barcode sequence, wherein the universal barcode sequence can be used for generating multiple barcodes. In some embodiments, the universal barcode sequence comprises two reference sites each located at both ends of the pin forming sequence. In some embodiments, a reference site is a nucleotide sequence of CCwGG (SEQ ID NO: 3). In some embodiments, the universal barcode sequence comprises one or more constant sites between the reference sites located at both ends of the pin forming sequence. In some embodiments, the universal barcode sequence comprises fourteen constant sites between the reference sites located at both ends of the pin forming sequence. In some embodiments, a constant site between two reference sites of a universal barcode sequence is a nucleotide sequence selected from CCwGG (SEQ ID NO: 3) and CawGG (SEQ ID NO: 54). In some embodiments, when propagated in bacteria, a constant site of a nucleotide sequence of CawGG (SEQ ID NO: 54) is not methylated, but can be easily methylated by changing to a nucleotide sequence of CCwGG (SEQ ID NO: 3). In some embodiments, the universal barcode sequence comprises fourteen constant sites between the reference sites located at both ends of the pin forming sequence. Accordingly, in such embodiments, a universal barcode sequence comprises a total of 14 potential methylation positions, which can be spaced randomly to avoid repetition. Accordingly, in such embodiments, a barcode can be generated by mutating any of the 14 CawGG (SEQ ID NO: 54) positions into CCwGG (SEQ ID NO: 3), and thus allowing a total of 214 possible combinations of barcode sequences.

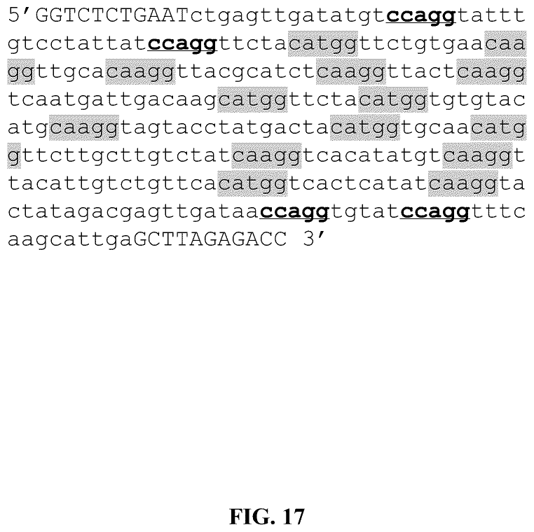

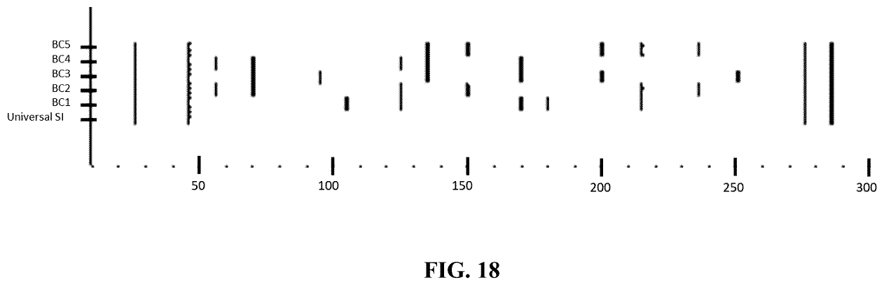

FIG. 17 shows an exemplary universal barcode sequence that allows to create subsequent barcode sequences by changing one or more of the 14 CawGG (SEQ ID NO: 54) positions into CCwGG. FIG. 18 shows exemplary barcode sequences generated as well as the fragments thereof, where only the four reference positions are methylated. Exemplary barcode sequences are provided in SEQ ID NOS: 39-44. An exemplary barcode sequence is provided in SEQ ID NO: 53.

In some embodiments, each of multiple hairpin nucleic acids comprise a barcode, wherein the barcode is uniquely associated with a nucleotide sequence of one of the multiple hairpin nucleic acids. In some embodiments, the barcode comprises one or more constant sites, wherein each of the multiple constant sites comprises a nucleotide sequence of CCwGG (SEQ ID NO: 3). In some embodiments, the multiple hairpin nucleic acids comprise at least two constant sites that are spaced differently relative to each other. In some embodiments, the multiple hairpin nucleic acids comprise at least two constant sites that are spaced differently relative to each other at either end of the fragment. In some embodiments, at least two of the multiple hairpin nucleic acids comprise a different number of constant sites relative to each other. Accordingly, in some embodiments, at least two of the multiple hairpin nucleic acids, upon methylation by DCM methylase in E. coli, comprises a pattern of 5-methylated cytosine modification(s) that is different relative to each other.

### Loop

In some embodiments, a first pin forming sequence 108 and a second pin forming sequence 109 of a hairpin nucleic acid 100 are linked to each other by a loop 110 to form a contiguous polynucleotide sequence. In some embodiments, the hairpin nucleic acid 100 comprises a loop 110 located between two pin forming sequences. In some embodiments, a size of the loop 110 is in a range of from 5 bases to 100 bases, from 5 bases to 80 bases, from 5 bases to 60 bases, from 5 bases to 40 bases, from 5 bases to 20 bases, from 10 bases to 100 bases, from 10 bases to 80 bases, from 10 bases to 60 bases, from 10 bases to 40 bases, from 10 bases to 20 bases, from 20 bases to 100 bases, from 20 bases to 80 bases, from 20 bases to 60 bases, from 20 bases to 40 bases, from 40 bases to 100 bases, from 40 bases to 80 bases, from 40 bases to 60 bases, from 60 bases to 100 bases, from 60 bases to 80 bases or from 80 bases to 100 bases.

In some embodiments, loops described herein comprise one or more contiguous nucleotides at 3′-end and one or more contiguous nucleotides at 5′-end, wherein the one or more contiguous nucleotides at 3′ end and the one or more contiguous nucleotides at 5′-end are complementary to each other. In some embodiments, the one or more contiguous nucleotides at 5′-end comprises at least 3, at least 4, at least 5, at least 10, at least 15 or at least 20 nucleotides. In some embodiments, the one or more contiguous nucleotides at 3′-end comprises at least 3, at least 4, at least 5, at least 10, at least 15 or at least 20 nucleotides. In some embodiments, the one or more contiguous nucleotides at 3′-end and the one or more contiguous nucleotides at 5′-end hybridized to each other resulting in a blunt end. In some embodiments, the one or more contiguous nucleotides at 3′-end and the one or more contiguous nucleotides at 5′-end hybridized to each other resulting in a sticky end. In some embodiments, the sticky end is located at the 3′ end or at the 5′ end. In some embodiments, the sticky end comprised at least 1, at least 2, at least 3, at least 4, or at least 5 single stranded bases. In some embodiments, the loop also comprised a stretch of polynucleotides located between the two complementary sequences (e.g., pin forming sequences) located at both end of the continuous sequence that is non-complementary, therefore forming a single stranded hinge. The hinge comprises at least 1, at least 2, at least 3, at least 4, at least 5, at least 10, at least 15 or at least 20 nucleotides. More preferably, this hinge comprises at least 2, at least 3, at least 4, or least 5, nucleotides. Even more preferably, this hinge comprises at least 4 nucleotides.

### Junction Sequence(s)

In some embodiments, intermediate portions of hairpin nucleic acids described herein comprise one or more junction sequences. In some embodiments, an intermediate portion 103 of a hairpin nucleic acid 100 comprises a first junction sequence 112, wherein the first junction sequence 112 is located between the first molecule binding sequence 104 and the first pin forming sequence 108. In some embodiments, an intermediate portion 103 of a hairpin nucleic acid 100 comprises a second junction sequence 113, wherein the second junction sequence 113 is located between the second pin forming sequence 109 and the second molecule binding sequence 105. In some embodiments, an intermediate portion 103 of a hairpin nucleic acid 100 comprises a first junction sequence 112 and a second junction sequence 113, wherein the first junction sequence 112 is located between the first molecule binding sequence 104 and the first pin forming sequence 108, and wherein the second junction sequence 113 is located between the second pin forming sequence 109 and the second molecule binding sequence 105. In some embodiments, the first junction sequence 112 is non-complementary to the second junction sequence 113. Accordingly, in some embodiments, the first spacer binding sequence comprising the first junction sequence 112 and the second spacer binding sequence comprising the second junction sequence 113 forms a 4-ways junction (or a Holiday junction) with a first spacer polynucleotide that is complementary to the first spacer binding sequence or a contiguous portion thereof, and wherein a second spacer polynucleotide that is complementary to the second spacer binding sequence or a contiguous portion thereof. In some embodiments, the first pin forming sequence 108 and the second pin forming sequence 109 are located between the first junction sequence 104 and the second junction sequence 105.

In some embodiments, junction sequences described herein comprise one or more contiguous nucleotides comprising at least 5, at least 10, at least 15, at least 20, at least 25, at least 30, at least 35 or at least 40 nucleotides. In some embodiments, junction sequences described herein comprise one or more contiguous nucleotides comprising at least 5, at least 10, at least 15, at least 20, at least 25, at least 30, at least 35 or at least 40 nucleotides.

In some embodiments, one or more contiguous nucleotides at 3′-end of a first junction sequence 112 and one or more contiguous nucleotides at 5′-end of a second junction sequence 113 are complementary to each other. In some embodiments, the one or more contiguous nucleotides at 3′-end of a first junction sequence 112 and the one or more contiguous nucleotides at 5′ end of a second junction sequence 113 hybridized to each other to form a blunt end. In some embodiments, the one or more contiguous nucleotides at 3′-end of a first junction sequence 112 and the one or more contiguous nucleotides at 5′-end of a second junction sequence 113 hybridized to each other to form a sticky end. In some embodiments, the sticky end is located at the 3′ end or at the 5′ end. In some embodiments, the sticky end comprised at least 1, at least 2, at least 3, at least 4, or at least 5 single stranded bases.

In some embodiments, one or more contiguous nucleotides at 3′-end of a first junction sequence 112 and one or more contiguous nucleotides at 5′-end of a second junction sequence 113 are complementary to each other. In some embodiments, the one or more contiguous nucleotides at 3′-end of a first junction sequence 112 and the one or more contiguous nucleotides at 5′ end of a second junction sequence 113 hybridized to each other to form a blunt end. In some embodiments, the one or more contiguous nucleotides at 3′-end of a first junction sequence 112 and the one or more contiguous nucleotides at 5′-end of a second junction sequence 113 hybridized to each other to form a sticky end.

### Adapter(s)

In some embodiments, the first ends of hairpin nucleic acids described herein comprise first adapters. In some embodiments, a first adapter 106 is configured to anchor a hairpin nucleic acid 100 to a bead. In some embodiments, the first adapter 106 covalently anchors the hairpin nucleic acid 100 to the bead. In some embodiments, the first adapter 106 non-covalently anchors the hairpin nucleic acid to the bead. In some embodiments, the first adapter 106 comprises a first nucleotide conjugating group that is configured to undergo an interaction (e.g., covalent interaction, non-covalent interaction or a combination thereof) with an anchoring molecule of the bead. In some embodiments, the first nucleotide conjugating group is selected from any one of DBCO group, azide group, tosyl group, amino group, sulfhydryl group, epoxy group, thiol group, hydroxyl group, chloromethyl group, streptavidin moiety or biotin moiety.

In some embodiments, a first adapter 106 is configured to anchor a hairpin nucleic acid 100 to a bead by hybridization. In some embodiments, the first adapter 106 comprises a polynucleotide sequence that is engineered to hybridize with an anchoring molecule, wherein the anchoring molecule comprises a polynucleotide sequence that is complementary to the first adapter 106. In some embodiments, the polynucleotide sequence of the anchoring molecule comprises at least a portion that is single stranded polynucleotide and that is configured to hybridize with the first adapter 106.

In some embodiments, second end of hairpin nucleic acids described herein comprises second adapters. In some embodiments, a second adapter 107 is configured to immobilize a hairpin nucleic acid 100 to a feature of a device. In some embodiments, the second adapter 107 covalently immobilizes the hairpin nucleic acid to the feature. In some embodiments, the second adapter 107 non-covalently immobilizes the hairpin nucleic acid to the feature. In some embodiments, the second adapter 107 comprises a second nucleotide conjugating molecule that is configured to undergo an interaction (e.g., covalent interaction, non-covalent interaction or a combination thereof) with the feature. In some embodiments, the second nucleotide conjugating molecule comprises a group selected from any one of DBCO group, azide group, tosyl group, amino group, sulfhydryl group, epoxy group, thiol group, hydroxyl group, chloromethyl group, streptavidin moiety or biotin moiety.

Alternatively, in some embodiments, a second adapter 107 is configured to immobilize a hairpin nucleic acid 100 to a feature of a device by hybridization. In some embodiments, the second adapter 107 comprises a polynucleotide sequence that is engineered to hybridize with the feature, wherein the feature comprises a polynucleotide sequence that is complementary to the second adapter 107. In some embodiments, the polynucleotide sequence of the feature comprises at least a portion that is single stranded and that is configured to hybridize with the second adapter 107.

Exemplary sequences of adaptors are provided in SEQ ID NOS: 11 and 36 for the bead anchoring oligonucleotide and SEQ ID NOS: 14 and 37 for the surface anchoring oligonucleotides.

### Spacer Polynucleotide

In some embodiments, systems described herein comprise one or more spacer polynucleotides. In some embodiments, a spacer polynucleotide described herein comprises a polynucleotide strand that has complementarity to a first spacer binding sequence or a second spacer binding sequence. In some embodiments, the spacer polynucleotide described herein comprises a polynucleotide strand that has complementarity to a first spacer binding sequence and a loop. In some embodiments, the spacer polynucleotide described herein comprises a polynucleotide strand that has complementarity to a loop and a second spacer binding sequence. In some embodiments, the spacer polynucleotide described herein comprises a polynucleotide strand that has complementarity to a first spacer binding sequence, a loop and a second spacer binding sequence. In some embodiments, the spacer polynucleotide after hybridization to a complementary spacer binding sequence prevents formation of a hairpin structure. Accordingly, in some embodiments, the spacer polynucleotide after hybridization to a complementary spacer binding sequence prevents hybridization between a first pin forming sequence and a second pin forming sequence. In some embodiments, the first and the second spacer polynucleotide are hybridized together to form a Y-shape through the complementary sequences on each polynucleotide.

In some embodiments, a spacer polynucleotide described herein comprises a Y-shaped (or substantially Y-shaped) spacer polynucleotide, wherein the Y-shaped spacer polynucleotide comprises two spacer polynucleotide strands (e.g., a first spacer polynucleotide and a second spacer polynucleotide), each comprising a pin forming sequence binding region and a junction sequence binding region. In some embodiments, pin forming sequence binding regions of the two spacer polynucleotides hybridizes with each other to form the Y-shaped spacer polynucleotide having a blunt end. In some embodiments, the pin forming sequence binding regions of the two spacer polynucleotides hybridize with each other to form the Y-shaped spacer polynucleotide having a sticky end. In some embodiments, the sticky end is located at the 3′ end or at the 5′ end. In some embodiments, the sticky end comprised at least 1, at least 2, at least 3, at least 4, or at least 5 single stranded bases. In some embodiments, the pin forming sequence binding regions of the two spacer polynucleotides are complementary to two pin forming sequences of the hairpin nucleic acid. In some embodiments, junction sequence binding regions of the two spacer polynucleotides are complementary to two junction sequences of the hairpin nucleic acid. In some embodiments, a portion of the junction sequence binding regions of the two spacer polynucleotide strands are complementary to portions of the two junction sequences of the hairpin nucleic acid. Accordingly, in some embodiments, the two spacer polynucleotides and the two junction sequences form the 4-way junction (or Holiday Junction). Alternatively, in some embodiments, a first spacer polynucleotide and a second spacer polynucleotide form a Y-shaped spacer polynucleotide that upon hybridization with a first junction sequence and a second junction sequence, respectively, of a hairpin nucleic acid 100 form a 4-way junction (or Holiday Junction). In some embodiments, the pin forming sequence binding regions of the two spacer polynucleotides are linked to each other by a spacer loop that is complementary to a loop of a hairpin nucleic acid 100. Alternatively, in some embodiments, the spacer polynucleotide comprises a polynucleotide sequence that is complementary to an intermediate portion 103 of a hairpin nucleic acid 100. In some embodiments, spacer polynucleotide described herein comprises a nucleotide sequence of SEQ ID NO: 12 or 13.

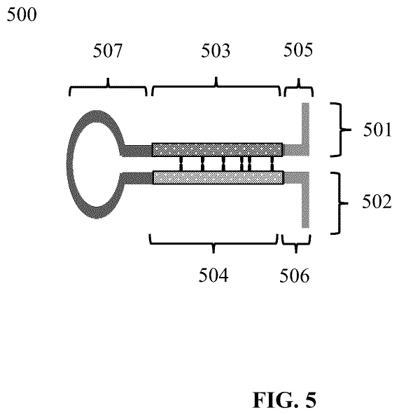

FIG. 5 shows exemplary structure of a spacer polynucleotide 500. As shown, the spacer polynucleotide 500 comprises a first spacer polynucleotide strand 501 and a second spacer polynucleotide strand 502. The first spacer polynucleotide strand 501 comprises a first junction sequence binding region 505 and a first pin forming sequence binding region 503. The second spacer polynucleotide strand 502 comprises a second junction sequence binding region 506 and a second pin forming sequence binding region 504. Also, as shown, the 5′-end of the first spacer polynucleotide strand 501 and the 3′-end of the second spacer polynucleotide strand 502 are linked to each other by a spacer loop 507.

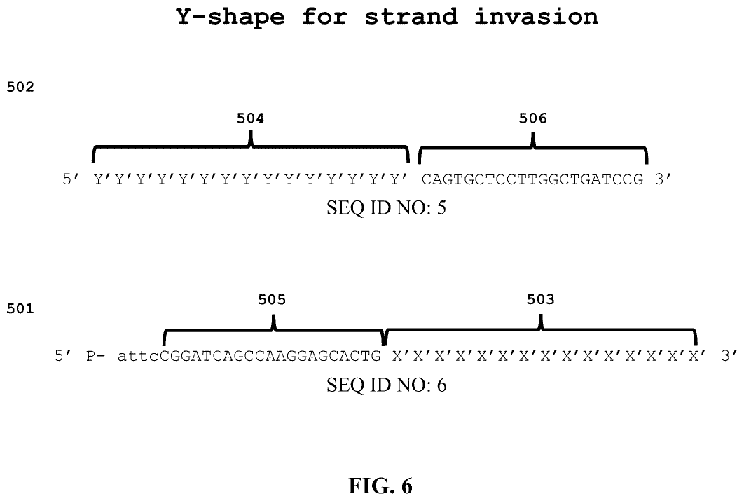

FIG. 6 shows non-limiting exemplary two spacer polynucleotides that can be used to make a Y-shaped spacer polynucleotide: (a) a first spacer polynucleotide strand 501 comprising a first junction sequence binding region 505 and a first pin forming sequence binding region 503 (from 5′ to 3′), and (b) a second spacer polynucleotide strand 502 comprising a second junction sequence binding region 504 and a second pin forming sequence binding region 506 (from 5′ to 3′).

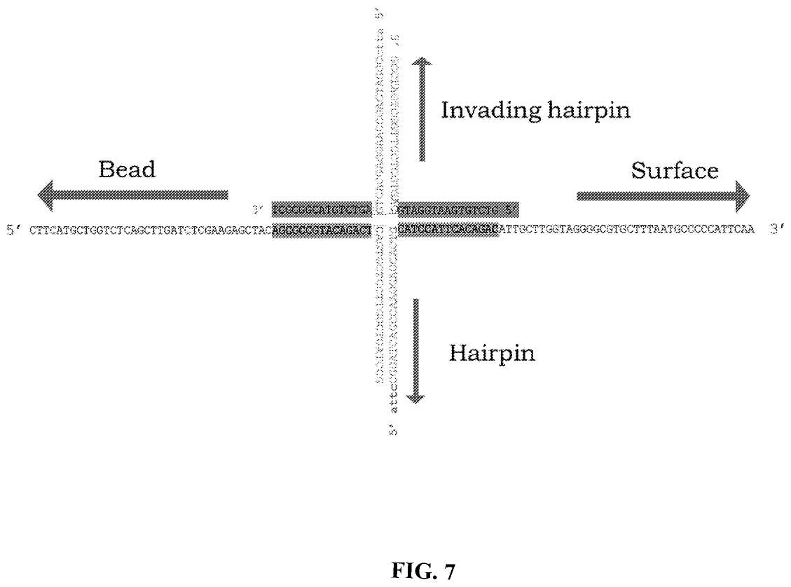

FIG. 7 shows a non-limiting exemplary system comprising a holiday junction that is formed between a nucleic acid scaffold and spacer polynucleotides. Upon application of a sufficient force, the Holiday junction can be resolved as the two pin sequences of both the molecule 100 and the space molecule 500 are complementary. In some embodiments, the sufficient force refers to a force in a range of from 5 pN to 10 pN.

### Candidate Molecule

In some embodiments, candidate molecules described herein are involved in or responsible for the etiology of a disease. In some embodiments, the candidate molecules are involved in preventing or treating a disease. In some embodiments, the candidate molecules are derived from a virus, a bacteria, a fungi or a mammalian cell. In some embodiments, the candidate molecules are isolated from a cell isolated from a cancer, from a pathological tissue, such as, a tumor or an amyloid plaque.

In some embodiments, the candidate molecule comprises a small molecule. In some embodiments, the small molecule comprises a molecule having a molecular weight in a range of from 50 Dalton to 20,000 Dalton. In some embodiments, the candidate molecule comprises a protein of interest. In some embodiments, the protein of interest comprises a polypeptide having at least two amino acids. In some embodiments, the candidate molecule comprises a nucleic acid of interest. In some embodiments, the nucleic acid of interest comprises a DNA, a RNA or a combination thereof. In some embodiments, the nucleic acid of interest comprises at least one conformational structure. A conformational structure of a nucleic acid (or a target nucleic acid) means a secondary or tertiary conformation, such as, for example, at least one of the conformations selected from a RNA hairpin, a P-shape RNA, a Y shape RNA, a candy shape RNA, and a combination thereof. In some embodiments, the nucleic acid of interest comprises an aptamer. In some embodiments, the aptamer comprises a DNA aptamer, a RNA aptamer, a XNA aptamer, or a combination thereof. In some embodiments, the nucleic acid of interest comprises a non-coding region of an RNA. In some embodiments, the nucleic acid of interest comprises a coding region of an RNA.

In some embodiments, the system described herein comprises a candidate molecule or a library of candidate molecules. In some embodiments, the library of candidate molecules comprising analogs and/or chemically modified analogs of the selected member(s) of the library of candidate molecules.

In some embodiments, candidate molecules disclosed herein are anchored to a hairpin nucleic acid 100. In some embodiments, the candidate molecules are anchored to the hairpin nucleic acid 100 by a covalent linkage. In some embodiments, the candidate molecules are anchored to the hairpin nucleic acid 100 by a non-covalent linkage. In some embodiments, the candidate molecules are anchored to the hairpin nucleic acid 100 by hybridization, wherein the candidate molecule is linked to a glue sequence, wherein the glue sequence is complementary to a molecule binding sequence of a hairpin nucleic acid 100. In some embodiments, the first glue sequence and the second glue sequence are not identical to each other. In some embodiments, a glue sequence described herein comprises an active group that is configured to link with a candidate molecule. Alternatively, in some embodiments, a glue sequence described herein comprises a first active group and a candidate molecule comprises a second active group, wherein the first and second active groups interact with each other for linking the glue sequence and the candidate molecule.

In some embodiments, a glue sequence described herein comprises a polynucleotide that is complementary to a small hairpin nucleic acid of a molecule binding sequence as described herein. FIG. 19C shows a graphical representation of a glue sequence comprising a polynucleotide that is complementary to a small hairpin nucleic acid of a molecule binding sequence.

In some embodiments, a glue sequence comprises a single stranded polynucleotide sequence 708. In some embodiments, a size of the glue sequence is in a range of from 5 bases to 100 bases, from 5 bases to 80 bases, from 5 bases to 60 bases, from 5 bases to 40 bases, from 5 bases to 20 bases, from 10 bases to 100 bases, from 10 bases to 80 bases, from 10 bases to 60 bases, from 10 bases to 40 bases, from 10 bases to 20 bases, from 20 bases to 100 bases, from 20 bases to 80 bases, from 20 bases to 60 bases, from 20 bases to 40 bases, from 40 bases to 100 bases, from 40 bases to 80 bases, from 40 bases to 60 bases, from 60 bases to 100 bases, from 60 bases to 80 bases or from 80 bases to 100 bases.

Systems described herein comprise a first candidate molecule and a second candidate molecule. In some embodiments, the first candidate molecule is anchored to a first molecule binding sequence 104 of a hairpin nucleic acid 100 by a first glue sequence. In some embodiments, the second candidate molecule is anchored to a second molecule binding sequence 105 of a hairpin nucleic acid 100 by a second glue sequence. Also, in some embodiments, systems described herein further comprise a third candidate molecule, wherein the third candidate molecule facilitates interaction between the first candidate molecule and the second candidate molecule. Alternatively, in some embodiments, systems described herein further comprise a third candidate molecule, wherein the third candidate molecule prevents interaction between the first candidate molecule and the second candidate molecule. In some embodiments, the third candidate molecule interacts with the first candidate molecule, the second candidate molecule or both. Accordingly, in some embodiments, an interaction between a third molecule and a first molecule facilitates/prevents interaction between the first molecule and a second molecule. In some embodiments, an interaction between a first molecule, a second molecule and a third molecule facilitates/prevents interaction between the first molecule and the second molecule.

### Beads

In some embodiments, beads disclosed herein are configured to anchor a hairpin nucleic acid 100 described herein by an anchoring molecule. In some embodiments, the anchoring molecule is covalently attached to a bead. In some embodiments, the beads comprise of non-conducting material (e.g., polymers, silicon, glass, resin, or a combination thereof).

In some embodiments, the beads comprise a diameter in a range of from 0.1 μm to 10 μm, from 0.3 μm to 10 μm, from 0.5 μm to 10 μm, from 1 μm to 10 μm, from 2 μm to 10 μm, from 5 μm to 10 μm, from 0.1 μm to 5 μm, from 0.3 μm to 5 μm, from 0.5 μm to 5 μm, from 1 μm to 5 μm, from 2 μm to 5 μm, from 0.1 μm to 2 μm, from 0.3 μm to 2 μm, from 0.5 μm to 2 μm, from 1 μm to 2 μm, from 0.1 μm to 1 μm, from 0.3 μm to 1 μm, from 0.5 μm to 1 μm, from 0.1 μm to 0.5 μm, from 0.3 μm to 0.5 μm or from 0.1 μm to 0.3 μm. In some embodiments, the magnetic bead comprises a diameter of 0.3 μm, 0.5 μm, 1.04 μm, 2.8 μm, or 5.5 μm.

In some embodiments, beads described herein comprise magnetic beads. In some embodiments, the magnetic beads (e.g., magnetic beads “MyOne”, produced by Invitrogen, having diameter of 1.04 μm; M270, produced by Invitrogen, of 2.8 μm diameter; M450, produced by Invitrogen, of 5.5 μm diameter; Ademtech 500, produced by Ademtech, of 0.5 μm diameter; Ademtech 300, produced by Ademtech, of 0.3 μm diameter) are configured to anchor first end of hairpin nucleic acids described herein.

Anchoring molecules described herein are configured to anchor hairpin nucleic acids (e.g., DNA, RNA, or a combination thereof) described herein to a bead. In some embodiments, a hairpin nucleic acid 100 is anchored onto a bead by an anchoring molecule. Alternatively, in some embodiments, a hairpin nucleic acid 100 is anchored to an anchoring molecule by an interaction (e.g., a covalent interaction or a non-covalent interaction). In some embodiments, the anchoring molecule is configured to form a covalent bond with a first adapter 106 of a first end of the hairpin nucleic acid. In some embodiments, the anchoring molecule comprises a group selected from any one of DBCO group, azide group, tosyl group, amino group, sulfhydryl group, epoxy group, thiol group, hydroxyl group, chloromethyl group, streptavidin moiety or biotin moiety. Accordingly, in some embodiments, the anchoring molecule and the first adapter 106 comprises a combination of groups that forms the covalent bond with each other, wherein the combination of groups comprise: (a) DBCO group and azide group; (b) tosyl group and amino group; (c) tosyl group and sulfhydryl group; (d) epoxy group and thiol group; (e) epoxy group and amino group; (f) epoxy group and hydroxyl group; or (g) chloromethyl group and amino group. Accordingly, in some embodiments, the first adapter 106 comprises DBCO group and the bead comprises azide group. Conversely, in some embodiments, the first adapter 106 comprises azide group and the bead comprises DBCO group. In some embodiments, the two groups having non-covalent interaction there between comprises a biotin moiety and a streptavidin moiety. Accordingly, in some embodiments, the first adapter 106 comprises a biotin moiety and the surface of the device comprises streptavidin moiety. Conversely, in some embodiments, the first adapter 106 comprises a streptavidin moiety and the bead comprises biotin moiety.

Alternatively, anchoring molecules described herein are configured to anchor hairpin nucleic acids described herein to a bead by hybridization. In some embodiments, a hairpin nucleic acid is anchored onto a bead by hybridization between a polynucleotide sequence of an anchoring molecule and a polynucleotide sequence of a first adapter 106, wherein the polynucleotide sequence of the anchoring molecule and the polynucleotide sequence of the first adapter 106 are complementary to each other, and wherein at least of portion of the polynucleotide sequence of the anchoring molecule is single stranded. In some embodiments, the single stranded portion of polynucleotide sequence of the anchoring molecule is at least 90%, at least 95%, at least 96%, at least 97%, at least 98%, at least 99% or 100% complementary to the polynucleotide sequence of the first adapter 106. In some embodiments, the single stranded portion of the polynucleotide sequence of the anchoring molecule comprises at least 5 bases, at least 10 bases, at least 15 bases, at least 20 bases, at least 25 pbases, at least 30 bases, at least 35 bases, at least 40 bases, at least 45 bases, at least 50 bases or more.

### Device

Devices disclosed herein comprise actuators, sensors, and chambers. In some embodiments, actuators are adapted to cause beads to move relative to the surface of the device in a predetermined direction. In some embodiments, the sensors are adapted to measure distances between the paramagnetic beads and the surface. In some embodiments, the chambers comprise an axis extending along the predetermined direction, the beads and a bottom surface. In some embodiments, the bottom surface comprises one or more features. In some embodiments, one or more mobilizing molecules are covalently attached to the feature.

In some embodiments, a device described herein comprises a force application mechanism (e.g., actuator). In some embodiments, actuators described herein are configured to generate a force. Accordingly, in some embodiments, the actuators are configured to move beads in the chamber using the force. In some embodiments, the force applied to the beads is constant. In some embodiments, the force applied to the beads is increased (ramp). Changes in the positions of the beads in the chamber are measured using the sensors of the device. In some embodiments, the force is a magnetic force. In some embodiments, the force is a force of laser radiation pressure.

In some embodiments, actuators described herein are configured to generate a magnetic force. Accordingly, in some embodiments, the actuators are configured to move beads in the chamber using the magnetic force. In some embodiments, the magnetic force applied to the beads is constant. In some embodiments, the magnet force applied to the beads is increased (ramp). Changes in the positions of the beads in the chamber are measured using the sensors of the device.

In some embodiments, actuators described herein are optical tweezers. Accordingly, in some embodiments, the optical tweezers are configured to trap beads in the chamber by using laser radiation pressure. In some embodiments, the laser radiation pressure force applied to the beads is constant. In some embodiments, the laser radiation pressure force applied to the beads is increased (ramp). Changes in the positions of the beads in the chamber are measured using the sensors of the device.

In some embodiments, the sensor comprises a camera (e.g., a CMOS camera), which is capable of collecting photons reflected from the paramagnetic beads. In some embodiments, the sensor comprises a CMOS capable of measuring impedance from the features caused by the movement of the beads up and down.

In some embodiments, devices described herein comprise: (a) a surface (e.g., silicon, glass, a non-conducting polymer or resin), configured to immobilize a second end of hairpin nucleic acids described herein; (c) an actuator, adapted to cause beads described herein to move relative to the surface of the device in one direction of motion; (d) a sensor, adapted to measure a distance between the bead and the surface; (e) a chamber, having an axis extending along the direction of motion of the bead and a bottom which is formed by the surface; and (f) an electrically conductive solution disposed in the chamber, where the electrically conductive solution comprises a conductivity of between 10⁻⁷ S/cm and 10¹ S/cm, or between 10⁻³ and 10⁻² S/cm, where the sensor is adapted to measure an impedance of the chamber, where the impedance is a function of the distance between the bead and the surface.

A device as described herein can include: a) an objective for collecting light radiations diffused by an object, the imaging system having an optical axis extending parallel to the first axis; b) a transmission mask having at least a first aperture and a second aperture, the first aperture and second aperture being spaced from each other along a second axis, perpendicular to the first axis, the transmission mask being arranged so as to let a first part of the radiations and a second part of the radiations which are diffused by the object pass through the first aperture and the second aperture respectively, while blocking a part of the radiations emitted by the light source which is not diffused by the object; and c) a detector adapted for generating an image including a first spot and a second spot representative of the first part and second part of the radiations impacting the detector plane, wherein variation of the position of the object relative to the object plane of the imaging system along the first axis causes variation of a position of the first spot and of the second spot relative to each other along the second axis.

In some embodiments, sensors described herein comprise: a main electrode, positioned on top of the chamber, in contact with the electrically conductive solution, the electrode being submitted to a known potential, a secondary electrode at the bottom of the chamber, carrying the surface to which the molecule can be attached, and an electronic circuit, adapted to measure a current flowing between the electrodes, that comprises a current to voltage amplifier connected to the secondary electrode, a voltmeter adapted to measure an output voltage of the current to voltage amplifier, and a computing circuit adapted to compute an impedance of the well from the measured voltage. In some embodiments, a sensor comprises a camera CMOS configured to collect photons reflected from the beads. In some embodiments, a sensor comprises a CMOS configured to measure impedance for a feature caused by the movement of the beads up and down.

In some embodiments, actuators described herein comprise at least a pair of actuators that are configured to control movement of a bead along the X-axis, Y-axis, Z-axis, or combinations thereof. In some embodiments, an actuator applies a constant force to a bead that allows controlling movement of a bead. In some embodiments, an actuator applies a variable force (e.g., increased force or decreased force) to a bead that allows controlling movement of a bead. In some embodiments, a force applied to a bead by an actuator is at least 0.01 pN, at least 0.1 pN, at least 1 pN, at least 2 pN, at least 3 pN, at least 5 pN, at least 10 pN, at least 20 pN, at least 40 pN, at least 60 pN or at least 95 pN. In some embodiments, a force applied to a bead by an actuator is not more than 100 pN. In some embodiments, a force necessary to control movement of a bead in the presence of candidate molecules is compared to a force necessary to control movement of the bead without candidate molecules. An example of force applied to a bead by an actuator, may be 0.1 to 35 pN, or more and up to 100 pN. In some embodiments, a force applied to a bead by an actuator may be 0.01 to 35 pN, or more and up to 100 pN.

In some embodiments, actuators described herein comprise at least a pair of magnets (e.g., permanent magnets, soft magnets, or combinations thereof) that are configured to control to move in translation along the X-X axis. In some embodiments, an actuator comprises two permanent magnets, positioned at equal distance of the X-X axis and having their magnetic poles aligned perpendicular to the X-X axis, the North pole of a magnet facing the South pole of the other. In some embodiments, a force of the magnet relative to the bead is constant. In some cases, the force of the magnet relative to the bead is increased or decrease by moving the permanent magnet relative to the beads. In some embodiments, the force of the magnet generated on the bead is at least 0.01 pN, at least 0.1 pN, at least 1 pN, at least 2 pN, at least 3 pN, at least 5 pN, at least 10 pN, at least 20 pN, at least 40 pN, at least 60 pN or at least 95 pN. In some embodiments, the force of the magnet generated on the beads is not more than 100 pN. The position of the beads in the presence of candidate molecules is compared to the position of the bead at the same force without candidate molecules. Alternatively, in some embodiments, the force of the magnet required to hold the bead at a particular position in the presence of candidate molecules is compared to the force required to hold the bead at the same position without candidate molecules. An example of force of the magnet relative to the bead, may be 0.1 to 35 pN, or more and up to 100 pN. In some embodiments, a force applied to a bead by an actuator may be 0.01 to 35 pN, or more and up to 100 pN. In some embodiments, at least a pair of magnets comprises more than one pair of magnets, more than five pair of magnets, more than ten pair of magnets, more than twenty pair of magnets, more than fifty pair of magnets, or more than hundred pair of magnets. In some embodiments, at least a pair of magnets comprises 1 to 500 pair of magnets, 1 to 400 pair of magnets, 1 to 300 pair of magnets, 1 to 200 pair of magnets, 1 to 100 pair of magnets, 1 to 50 pair of magnets, 1 to 10 pair of magnets, 1 to 5 pair of magnets, 50 to 500 pair of magnets, 50 to 400 pair of magnets, 50 to 300 pair of magnets, 50 to 200 pair of magnets, 50 to 100 pair of magnets, 100 to 500 pair of magnets, 100 to 400 pair of magnets, 100 to 300 pair of magnets, 100 to 200 pair of magnets, 200 to 500 pair of magnets, 200 to 400 pair of magnets, 200 to 300 pair of magnets, 300 to 500 pair of magnets, 300 to 400 pair of magnets, or 400 to 500 pair of magnets.

As disclosed herein, a feature means a binding moiety on a surface of a device described herein. Accordingly, in some embodiments, the feature is configured to immobilize hairpin nucleic acids (e.g., DNA, RNA, or a combination thereof) described herein to the surface of the device. Immobilize means fixed by one side to the surface of the device and does not prevent from moving. Alternatively, in some embodiments, a hairpin nucleic acid is immobilized to the surface of the device by an interaction (e.g., a covalent interaction or a non-covalent interaction) with the feature. In some embodiments, the feature is configured to form a covalent bond with a second adapter 107 of a second end of the hairpin nucleic acid. In some embodiments, the feature comprises a group selected from any one of DBCO group, azide group, tosyl group, amino group, sulfhydryl group, epoxy group, thiol group, hydroxyl group, chloromethyl group, streptavidin moiety or biotin moiety. Accordingly, in some embodiments, the feature and the second adapter 107 comprise a combination of groups that forms the covalent bond with each other, wherein the combination of groups comprise: (a) DBCO group and azide group; (b) tosyl group and amino group; (c) tosyl group and sulfhydryl group; (d) epoxy group and thiol group; (e) epoxy group and amino group; (f) epoxy group and hydroxyl group; or (g) chloromethyl group and amino group. Accordingly, in some embodiments, the second adapter 107 comprises DBCO group and the feature comprises azide group. Conversely, in some embodiments, the second adapter 107 comprises azide group and the feature comprises DBCO group. In some embodiments, the two groups having non-covalent interaction there between comprises a biotin moiety and a streptavidin moiety. Accordingly, in some embodiments, the second adapter 107 comprises a biotin moiety and the feature comprises streptavidin moiety. Conversely, in some embodiments, the second adapter 107 comprises a streptavidin moiety and the feature comprises biotin moiety.

In some embodiments, a feature of a surface of a device is configured to immobilize hairpin nucleic acids described herein by hybridization. In some embodiments, a hairpin nucleic acid is immobilized onto a surface of a device by hybridization between a polynucleotide sequence of a feature of the surface and a polynucleotide sequence of a second adapter 107, wherein the polynucleotide sequence of the feature and the polynucleotide sequence of the second adapter 107 are complementary to each other, and wherein at least of portion of the polynucleotide sequence of the feature is single stranded. In some embodiments, the single stranded portion of polynucleotide sequence of the feature is at least 90%, at least 95%, at least 96%, at least 97%, at least 98%, at least 99% or 100% complementary to the polynucleotide sequence of the second adapter 107. In some embodiments, the single stranded portion of the polynucleotide sequence of the feature comprises at least 5 bases, at least 10 bases, at least 15 bases, at least 20 bases, at least 25 bases, at least 30 bases, at least 35 bases, at least 40 bases, at least 45 bases, at least 50 bases or more.

As disclosed herein, a device can comprise a plurality of features. For examples, a device can comprise at least 1, at least 10, at least 100, at least 1000, at least 2000, at least 3000, at least 4000, at least 5000, at least 6000, at least 7000, at least 8000, at least 9000, at least 10000, at least 20000, at least 30000, at least 40000, at least 50000, at least 60000, at least 70000, at least 80000, at least 90000, at least 100000, at least 500000, at least 1000000, at least 2000000, at least 5000000 or greater than 10000000 features. Accordingly, in some embodiments, a feature, as described herein, represents at least 1,000 features. Accordingly, in some embodiments, a feature, as described herein, represents at least 2,000,000 features. As such, individual binding experiments can be performed on a vast number of individual molecules simultaneously.

In some embodiments, a feature is covalently and/or non-covalently attached to a surface of a device. Covalent attachment of the feature to the surface of the device can be performed by a skilled person (e.g., either esterification, click chemistry). In some embodiments, the surface of the device comprises at least two features. In some embodiments, the at least two features are same relative to each other. In some embodiments, the at least two features are different relative to each other. In some embodiments, having different features is advantageous to anchor different hairpin nucleic acids at specific position within the device. This allows to map rapidly the different hairpin nucleic acids within the device.

### Nucleic Acid Scaffold

Disclosed herein are nucleic acid scaffolds. In some embodiments, a nucleic acid scaffolds comprise a hairpin nucleic acid 100, a first spacer polynucleotide hybridized to a first spacer binding sequence of the hairpin nucleic acid 100, a second spacer polynucleotide hybridized to a second spacer binding sequence of the hairpin nucleic acid 100. In some embodiments, the nucleic acid scaffold is anchored to a bead by attaching a first end 101 of the hairpin nucleic acid 100 to the bead. In some embodiments, the nucleic acid scaffold is immobilized to a feature of a surface of a device by attaching a second end 102 of the hairpin nucleic acid 100 to the feature. In some embodiments, the spacer polynucleotides are hybridized to first junction sequence 112 and second junction sequence 113 of the nucleic acid scaffold. In some embodiments, the spacer polynucleotides, upon binding with the first junction forming sequence 112, the second junction forming sequence 113, the first pin forming sequence 108, the second pin forming sequence 109, and the loop 110 prevents formation of a hairpin structure and form a nucleic acid scaffold. In some embodiments, a length of the nucleic acid scaffold is a reference elongation length. In some embodiments, the reference elongation length is determined when a magnetic force applied to a magnetic bead, wherein the nucleic acid scaffold is immobilized between the magnetic bead and a surface of the device. In some embodiments, the reference elongation length is determined at 0.2 pN magnetic force applied to a magnetic bead, wherein the nucleic acid scaffold is immobilized between the magnetic bead and a surface of the device. In some embodiments, the reference elongation length is adjusted by changing a size of the hairpin nucleic acid.

In some embodiments, a first molecule binding sequence 104 of the nucleic acid scaffold and a second molecule binding sequence 105 of the nucleic acid scaffold upon positioning of a first candidate molecule and a second candidate molecule, respectively, form a screening nucleic acid scaffold. In some embodiments, an elongation length of the screening nucleic acid scaffold is less than the reference elongation length when magnetic force of 0.1 pN to 50 pN, 0.1 pN to 40 pN, 0.1 pN to 30 pN, 0.1 pN to 20 pN, 0.1 pN to 10 pN, 0.2 pN to 50 pN, 0.2 pN to 40 pN, 0.2 pN to 30 pN, 0.2 pN to 20 pN, 0.2 pN to 10 pN, 0.5 pN to 50 pN, 0.5 pN to 40 pN, 0.5 pN to 30 pN, 0.5 pN to 20 pN, 0.5 pN to 10 pN, 1 pN to 50 pN, 1 pN to 40 pN, 1 pN to 30 pN, 1 pN to 20 pN, or 1 pN to 10 pN, 10 pN to 50 pN, 10 pN to 40 pN, 10 pN to 30 pN, or 10 pN to 20 pN is applied to the magnetic bead. In some embodiments, an elongation length of the screening nucleic acid scaffold is less than the reference elongation length when a magnetic force is applied to the magnetic bead. In some embodiments, an elongation length of the screening nucleic acid scaffold is less than the reference elongation length when 0.2 pN magnetic force is applied to the magnetic bead.

In some embodiments, a screening nucleic acid scaffold as described herein is incubated with a third candidate molecule resulting in change in elongation length of the screening nucleic acid scaffold or change in force required to achieve same elongation length as the reference elongation length. In some embodiments, the third candidate molecule binds to the first candidate molecule, the second candidate molecule or both. In some embodiments, the third candidate molecule facilitates binding of the first candidate molecule and the second candidate molecule causing to decrease elongation length of the screening nucleic acid scaffold or requiring more force to achieve the same elongation length as the reference elongation length. Conversely, in some embodiments, the third candidate molecule hinders binding of the first candidate molecule and the second candidate molecule causing an elongation length of the screening nucleic acid scaffold similar to the reference elongation length without the molecule or requiring less force to achieve the same elongation length as the reference elongation length.

### Electrically Conductive Solution

In some embodiments, the systems described herein comprise an electrically conductive solution disposed in the chamber described herein. In some embodiments, the chamber comprises a sensor that is adapted to measure an impedance of the chamber. In some embodiments, the impedance is a function of the distance between the bead and the feature (e.g., surface). In some embodiments, the electrically conductive solution can have a conductivity of between 10⁻⁷ S/cm and 10¹ S/cm, or between 10⁻³ and 10⁻² S/cm.

### Kits

Also disclosed herein are kits comprising one or more components of the systems disclosed herein. In some embodiments, the one or more components comprise hairpin nucleic acids described herein, spacer polynucleotides described herein, candidate molecules described herein, beads described herein, and glue sequences described herein. In some embodiments, the one or more components comprise hairpin nucleic acids with at least one barcode comprising a modified basis described herein, an antibody specific for the modified basis of the barcode, spacer polynucleotides described herein, candidate molecules described herein, beads described herein, and glue sequences described herein. In some embodiments, the kit is configured to form a nucleic acid scaffold, wherein the nucleic acid scaffold comprises a hairpin nucleic acid 100 and spacer polynucleotides. In some embodiments, the kit is configured to form a nucleic acid scaffold, wherein the nucleic acid scaffold comprises no double-stranded DNA molecule comprising a first double-stranded DNA molecule (1) connected to a second double-stranded DNA molecule (2) by a tether comprising double-stranded DNA, wherein the tether is attached by (i) at least one covalent bond to a nucleotide of the first double-stranded DNA molecule (1), and by (ii) at least one covalent bond to a nucleotide of the second double-stranded DNA molecule (2). In some embodiments, the nucleic acid scaffold further comprises one or more candidate molecules described herein. In some embodiments, the kit is configured to determine a binding interaction between two or more candidate molecules. In some embodiments, the kit is configured to screen the candidate molecule having a binding interaction with one or more candidate molecules from a library of candidate molecules, wherein a force of the binding interaction is in a range of from 0.01 pN to 100 pN, or from 0.1 pN to 100 pN. In some embodiments, the kit is configured to screen the candidate molecule having a force of the binding interaction in a range of from 0.01 pN to 5 pN, or from 0.1 pN to 5 pN.

In some embodiments, a kit can comprise a container and instructions for use. In some embodiments, the kit further comprises a library of candidate molecules. In some embodiments, the library of candidate molecules comprises a library of small molecules, a library of proteins, a library of antibodies, a library of aptamers, advantageously a library comprising binding molecules. In some embodiments, the kit further comprises a buffer comprising a monovalent cation in a range of from 1 nM to 1 M. In some embodiments, the monovalent cation comprises Na⁺, K⁺, Li⁺, or a combination thereof. In some embodiments, the buffer comprises about 500 mM Na⁺, about 150 mM Na⁺, about 137 mM Na⁺, about 10 mM Na⁺, about 1 mM Na⁺, about 500 μM Na⁺, about 100 μM Na⁺, about 10 μM Na⁺, about 1 μM Na⁺, about 500 nM Na⁺, about 100 nM Na⁺, about 10 nM Na⁺, or about 1 nM Na⁺. In some embodiments, the buffer comprises about 150 mM Na⁺. In some embodiments, the buffer comprises about 137 mM Na⁺. In some embodiments, the buffer contains about 500 mM K⁺, about 150 mM K⁺, about 137 mM K⁺, about 13 mM K⁺, about 10 mM K⁺, about 1 mM K⁺, about 500 μM K⁺, about 100 μM K⁺, about 10 μM K⁺, about 1 μM K⁺, about 500 nM K⁺, about 100 nM K⁺, about 10 nM K⁺, or about 1 nM K⁺. In some embodiments, the buffer comprises about 150 mM K⁺. In some embodiments, the buffer comprises about 13 mM K⁺. In some embodiments, the buffer comprises about 137 mM Na⁺ and about 13 mM K⁺. In some embodiments, the kit further comprises a buffer comprises a divalent cation in a range of from 1 μM to 1 mM. In some embodiments, the divalent cation comprises Mg²⁺, Mn²⁺, Zn²⁺, Fe²⁺, Cu²⁺, Cd²⁺, or a combination thereof. In some embodiments, the buffer comprises less than 10 mM Mg²⁺, less than 5 mM Mg²⁺, advantageously >0.01 mM Mg²⁺ and less than 10 mM Mg²⁺, more advantageously 1 mM Mg²⁺.

### Methods for Making a Hairpin Nucleic Acid

Disclosed herein are methods of making a hairpin nucleic acid 100, wherein the hairpin nucleic acid 100 comprises a first end 101 as described herein, an intermediate portion 103 as described herein, and a second end 102 as described herein. In some embodiments, the methods comprise: (a) providing a first hairpin nucleic acid precursor comprising a first end of the first hairpin nucleic acid precursor and a second end of the first hairpin nucleic acid precursor, wherein the first hairpin nucleic acid precursor is a double stranded polynucleotide, wherein each strand of the first hairpin nucleic acid precursor comprises a pin forming sequence; (b) ligating a loop to the first end of the first hairpin nucleic acid precursor to form a second hairpin nucleic acid precursor; (c) ligating a first Y-shape forming polynucleotide encoding one or more of the first end 101 and a first junction sequence 112 of the hairpin nucleic acid 100, and a second Y-shape forming polynucleotide encoding one or more of the second end 102 and a second junction sequence 113 of the hairpin nucleic acid 100 to the second hairpin nucleic acid precursor to form the hairpin nucleic acid 100. In some embodiments, the ligated product is purified/recovered by any suitable method (e.g., gel purification).

In some embodiments, a first hairpin nucleic acid precursor is chemically synthesized. In some embodiments, the first hairpin nucleic acid precursor is synthesized in vivo (e.g., E. coli). For example, in vivo synthesis of the first hairpin nucleic acid precursor comprises: (a) cloning the first hairpin nucleic acid into a plasmid; (b) propagating the plasmid in a host (e.g., E. coli); (c) recovering plasmid from the host; (d) digesting a recovered plasmid with the restriction enzyme to form the first hairpin nucleic acid precursor.

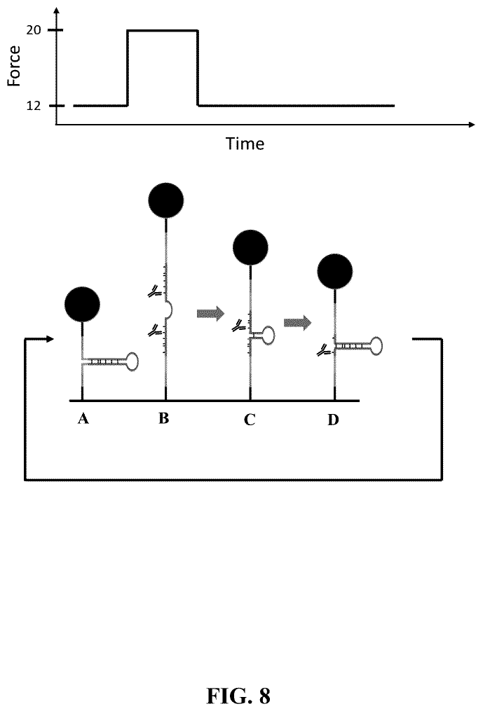

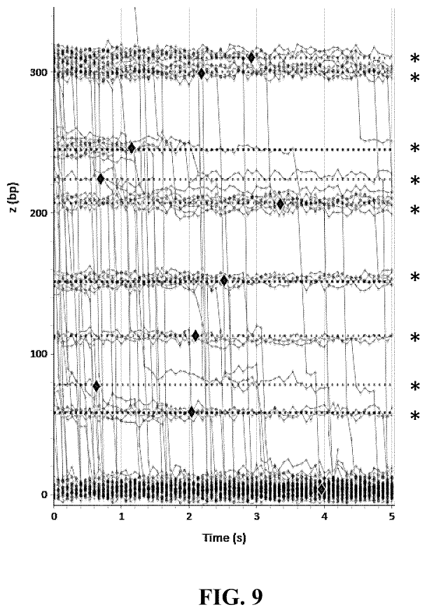

In some embodiments, a first hairpin nucleic acid precursor comprises pin forming sequence. Accordingly, in some embodiments, the first hairpin nucleic acid precursor further comprises a barcode. In some embodiments, the barcode comprises one or more nucleotides that undergo epigenetic modifications. For example, in some embodiments, the barcode comprises one or more cytosines that undergo epigenetic modifications (e.g., methylation). In some embodiments, the barcode comprises one or more constant sites (e.g., CCwGG (SEQ ID NO: 3)) spaced within the first hairpin nucleic acid precursor. Accordingly, in some embodiments, where the first hairpin nucleic acid precursor is propagated in E. coli, the first hairpin nucleic acid precursor comprises one or more methylated cytosines. In some embodiments, the barcode comprises two constant sites (e.g., CCwGG (SEQ ID NO: 3)) spaced within the first hairpin nucleic acid precursor. In some embodiments, the two constant sites are present in the first hairpin nucleic acid precursor are spaced by 18 and 10 bases on either end of the first hairpin nucleic acid precursor. Alternatively, in some embodiments, the two constant sites are present in the first hairpin nucleic acid precursor are spaced by 20 and 10 bases on either end of the first hairpin nucleic acid precursor. FIG. 8 shows a graphical representation of method for identifying hairpin nucleic acids. As shown, the graphical representation provides methods to identify methylated cytosines (5mC) using anti-5mC antibody. The number of 5mC detected per cycle depends on the concentration of the anti-5mC antibody. In some embodiments, the antibody is injected at a concentration that allow detection of 1 to 6, 1 to 5, 1 to 4, 1 to 3, 2 to 6, 2 to 5, 2 to 4, 2 to 3, 3 to 6, 3 to 5, 3 to 4, 4 to 6, 4 to 5, or 5 to 6 methylated cytosine sites per cycle in a universal barcode sequence of a hairpin nucleic acid. Therefore, the cycle can be repeated many times until all the positions of the universal barcode sequence that have 5mC modifications are extracted. In some embodiments, a universal barcode sequence of a hairpin nucleic acid described herein has at least 18, at least 16, at least 14, at least 12, at least 10, at least 8 sites, at least 6 sites, at least 4 sites, at least two sites or at least one methylated cytosine. FIGS. 9 and 21 show exemplary barcode sequences having constant sites at different positions. In some embodiments other barcode sequences comprising alternative methylated bases can be prepared in various hosts and used with a specific antibody. These are part(s) of the kit provided here.

### Methods for Making a Spacer Polynucleotide

Disclosed herein are methods of making a spacer polynucleotide hairpin as described herein. In some embodiments, the methods comprise: (a) providing a first hairpin nucleic acid precursor comprising a first end and a second end, wherein the first hairpin nucleic acid precursor is a double stranded polynucleotide, wherein each strand of the first hairpin nucleic acid precursor comprises a pin forming sequence; (b) ligating a first Y-shape forming polynucleotide encoding a complementary sequence to the first junction sequence 112, and a second Y-shape forming polynucleotide encoding acomplementary sequence of the second junction sequence 113 at the first end of the double stranded first hairpin nucleic acid precursor to form the spacer polynucleotide. In some embodiments, the spacer polynucleotide comprises a blunt end or a sticky end. In some embodiments, the methods further comprise ligating a spacer loop to the blunt end or the sticky end. In some embodiments, the Y-shaped spacer polynucleotide is complementary to a Y-shaped hairpin nucleic acid.

### Methods for Making Nucleic Acid Scaffolds

Disclosed herein are methods and systems for making nucleic acid scaffolds. In some embodiments, the methods comprise: (a) providing a hairpin nucleic acid 100 as described herein, wherein the hairpin nucleic acid 100 is a contiguous polynucleotide sequence; (b) attaching a bead to a first end 101 of the hairpin nucleic acid 100; and (c) attaching a second end 102 of the hairpin nucleic acid 100 to a bottom surface of a device; (d) hybridizing a first spacer polynucleotide to a first spacer binding sequence of the hairpin nucleic acid 100, wherein the first spacer polynucleotide comprises a polynucleotide sequence that is complementary to at least a portion of the first spacer binding sequence; (e) hybridizing a second spacer polynucleotide to a second spacer binding sequence of the hairpin nucleic acid 100, wherein the second spacer polynucleotide comprises a polynucleotide sequence that is complementary to at least a portion of the second spacer binding sequence. In some embodiments, a first spacer polynucleotide and a second spacer polynucleotide are provided together, wherein the first spacer polynucleotide comprises a polynucleotide sequence that is complementary to at least a portion of a first spacer binding sequence of the hairpin nucleic acid 100, wherein the second spacer polynucleotide comprises a polynucleotide sequence that is complementary to at least a portion of a second spacer binding sequence of the hairpin nucleic acid 100, and wherein the first spacer polynucleotide hybridizes to the first spacer binding sequence and the second spacer polynucleotide hybridizes to the second spacer binding sequence. In some embodiments, the scaffolds described herein exclude a double-stranded DNA molecule comprising a first double-stranded DNA molecule connected to a second double-stranded DNA molecule (2) by a tether comprising double-stranded DNA, wherein the tether is attached (i) at least one covalent bond to a nucleotide of the first double-stranded DNA molecule (1), and b (ii) at least one covalent bond to a nucleotide of the second double-stranded DNA molecule (2).

Also, disclosed herein are methods and systems for forming a plurality of nucleic acid scaffolds. In some embodiments, the methods comprise: (a) providing a plurality of hairpin nucleic acids, wherein each of the plurality of hairpin nucleic acids is a contiguous polynucleotide sequence, wherein each of the plurality of hairpin nucleic acids comprises a barcode, wherein at least two of the plurality of hairpin nucleic acids comprises a different barcode; (b) attaching a bead to a first end of each of the plurality of hairpin nucleic acids; and (c) attaching a second end of each of the plurality of hairpin nucleic acids to a bottom surface of a device; (d) decoding the barcode for each of the plurality of hairpin nucleic acid; (e) hybridizing a first spacer polynucleotide to each of a first spacer binding sequence of the plurality of hairpin nucleic acids, wherein the first spacer polynucleotide comprises a polynucleotide sequence that is complementary to the first spacer binding sequence; (f) hybridizing a second spacer polynucleotide to each of a second spacer binding sequence of the plurality of hairpin nucleic acids, wherein the second spacer polynucleotide comprises a polynucleotide sequence that is complementary to the second spacer binding sequence. In some embodiments, a first spacer polynucleotide and a second spacer polynucleotide are provided together, wherein the first spacer polynucleotide comprises a polynucleotide sequence that is complementary to at least a portion of a first spacer binding sequence of the plurality of hairpin nucleic acids, wherein the second spacer polynucleotide comprises a polynucleotide sequence that is complementary to at least a portion of a second spacer binding sequence of the plurality of hairpin nucleic acids, and wherein the first spacer polynucleotide hybridizes to the first spacer binding sequence and the second spacer polynucleotide hybridizes to the second spacer binding sequence.

In some embodiments, the decoding of the barcode provides identity of underlying nucleotide sequence of the hairpin nucleic acid 100. Accordingly, in some embodiments, the decoding allows to identify a first molecule binding sequence 104 and a second molecule binding sequence 105 of the hairpin nucleic acid, wherein a first candidate molecule and a second candidate molecule are anchored to the first molecule binding sequence 104 and the second molecule binding sequence 105, respectively. In some embodiments, at least one of the first molecule binding sequence 104 and the second molecule binding sequence 105 is a small hairpin nucleic acid. In some embodiments, both, the first molecule binding sequence 104 and the second molecule binding sequence 105, are small hairpin nucleic acids.

For making the nucleic acid scaffolds, in some embodiments, the hairpin nucleic acids are linked to beads. The beads linked with the hairpin nucleic acids are injected into the flow cell for immobilizing them to features of the flow cell. The immobilizing is achieved by hybridizing second adapter sequences of the hairpin nucleic acids with the features having a complementary nucleotide sequence. Features having complementary polynucleotide sequence (SEQ ID NO: 24) can be attached to the surface of the flow cell by covalent interaction (e.g., click chemistry between azide coated bottom surface of the flow cell and DBCO end group containing polynucleotide sequence). Non-attached beads cand be washed away.

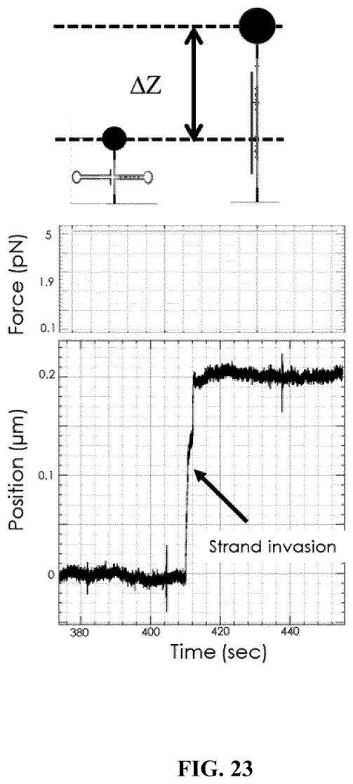

FIGS. 11 and 22 show a graphical representation of method of making nucleic acid scaffold from a hairpin nucleic acid positioned between a bead and a feature of a surface of a device. Briefly, as shown in FIG. 11, A, the hairpin nucleic acid 100 is then incubated with spacer polynucleotides to form a holiday junction. Followingly, as shown in FIG. 11, B, a sufficient force is applied to resolve holiday junction, which results in strand invasion and formation of the nucleic acid scaffold. As shown in FIG. 11, C, upon release of the force, two spacer binding sequences of the nucleic acid scaffold are not hybridized to each other. Alternatively, as shown in FIG. 22, A, the hairpin nucleic acid 100 is incubated with a spacer polynucleotide to form a holiday junction. Followingly, as shown in FIG. 22, B, a sufficient force is applied to resolve holiday junction, which results in strand invasion and formation of the nucleic acid scaffold. As shown in FIG. 22, C, upon release of the force, two spacer binding sequences of the nucleic acid scaffold are not hybridized to each other. FIG. 23 shows an exemplary trace of events described in FIG. 22. A hairpin nucleic acid comprising a universal barcode sequence can be used for generating a nucleic acid scaffold by allowing strand invasion. In some embodiments, one or more mutations present in the barcode sequence of a hairpin nucleic acid generated from the universal barcode sequence does not affect strand invasion efficiency of a spacer nucleotide(s).

Methods for Determining Binding Interactions and Binding Energies Associated with Binding Interactions

Disclosed herein are methods and systems for use in determining binding interactions and binding energies associated with binding interactions between at least two candidate molecules. In some embodiments, at least two candidate molecules are selected proteins of interest described herein, nucleic acids of interest described herein, and small molecules of interest described herein. In some embodiments, at least two candidate molecules are proteins of interest. In some embodiments, at least two candidate molecules are small molecules of interest. In some embodiments, at least two candidate molecules are nucleic acids of interest. In some embodiments, at least two candidate molecules are selected from proteins of interest and nucleic acids of interest. In some embodiments, at least two candidate molecules are selected from proteins of interest and small molecules of interest. In some embodiments, at least two candidate molecules are selected from nucleic acids of interest and small molecules of interest.

Disclosed herein are methods and systems for determining binding interactions between candidate molecules in real time. Also, disclosed herein are methods and systems for determining binding energies associated with binding interactions between candidate molecules. In some embodiments, the methods and systems are capable of detecting binding interaction between candidate molecules in real time and analysing molecular interactions between the candidate molecules. In some embodiments, the methods and systems comprise determining binding interactions between candidate molecules at a single molecular level. In some embodiments, the methods and systems comprise determining at least two binding interactions in parallel between candidate molecules at a single molecular level. In some embodiments, the methods and systems comprise use of magnetic tweezers for determining binding interactions between candidate molecules. In some embodiments, the methods and systems comprise use of optical tweezers for determining binding interactions between candidate molecules.

Disclosed herein are methods and systems for determining binding interactions (Kon and Koff) between a first candidate molecule and a second candidate molecule using a nucleic acid scaffold. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) determining a reference amplitude of the Brownian noise of the nucleic acid scaffold in response to a low force (<0.01 pN) in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule can be in close proximity or in contact with each other; (f) determining an amplitude of the Brownian noise of the screening nucleic acid scaffold in response to the same force used in the absence of the first candidate molecule and the second candidate molecule by repeating (d); and (g) identify events of interaction between the two molecules, wherein the amplitude of the Brownian noise is reduced compared to the reference amplitude at the same force in the absence of the molecules. A value corresponding to about ⅓ the value of the amplitude indicates presence of the binding interaction between the first candidate molecule and the second candidate molecule at the force applied. Once these events are identified, in some embodiments, the Kon is determined by taking into consideration all the events over the time spend when the two molecules don't interact and the Koff corresponds to the average time of all these events detected. In some embodiments, the first molecule binding sequence comprise a small hairpin nucleic acid, and the first glue sequence comprises a polynucleotide that is complementary to the small hairpin nucleic acid. In some embodiments, the second molecule binding sequence comprise a small hairpin nucleic acid, and the second glue sequence comprises a polynucleotide that is complementary to the small hairpin nucleic acid.

Also, disclosed herein are methods and systems for determining binding interactions (Kon and Koff) between a first candidate molecule, a second candidate molecule and a third candidate molecule using a nucleic acid scaffold. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) determining a reference amplitude of the Brownian noise of the nucleic acid scaffold in response to a low force (<0.01 pN) in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact within each other; (f) contacting a third candidate molecule in solution to the screening nucleic acid scaffold; (g) determining an amplitude of the Brownian noise of the screening nucleic acid scaffold in the presence of the third candidate molecule in response to the same low force applied in (d); (h) identify events of interaction between the three molecules, wherein the amplitude of the Brownian noise is reduced compared to the reference amplitude at the same force in the absence of the molecules. A value corresponding to about ⅓ the value of the amplitude indicates presence of the binding interaction between the first candidate molecule, the second candidate molecule and the third molecule in solution at the force applied. Once these events are identified, it is possible to determine the Kon by taking into consideration all the events over the time spend when the two molecules don't interact and the Koff corresponds to the average time of all these events detected. In some embodiments, the first molecule binding sequence comprise a small hairpin nucleic acid, and the first glue sequence comprises a polynucleotide that is complementary to the small hairpin nucleic acid. In some embodiments, the second molecule binding sequence comprise a small hairpin nucleic acid, and the second glue sequence comprises a polynucleotide that is complementary to the small hairpin nucleic acid.

Also, disclosed herein are methods and systems for determining binding interactions (Kₒₙ and K_{off}) between a first candidate molecule, a second candidate molecule and a third candidate molecule using a nucleic acid scaffold. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact within each other; (e) determining a reference amplitude of Brownian noise of the nucleic acid scaffold in response to a force in the presence of the first candidate molecule and the second candidate molecule; (f) contacting a third candidate molecule in solution to the screening nucleic acid scaffold; (g) determining an amplitude of Brownian noise of the screening nucleic acid scaffold in the presence of the third candidate molecule in response to the same low force applied in (e); (h) identify events of interaction between the three molecules, wherein the amplitude of Brownian noise is reduced or remained unchanged compared to the reference amplitude at the same force in the presence of the first and second molecules. A reduction in Browning noise in the presence of the third molecule indicates that the third molecule stabilizes interaction between the first and the second molecule. In contrast, if the amplitude of Brownian noise remains unchanged in the presence of the third molecule indicates that the third molecule did not affect interactions between the first and the second molecules. In some embodiments, the first molecule binding sequence comprise a small hairpin nucleic acid, and the first glue sequence comprises a polynucleotide that is complementary to the small hairpin nucleic acid. In some embodiments, the second molecule binding sequence comprise a small hairpin nucleic acid, and the second glue sequence comprises a polynucleotide that is complementary to the small hairpin nucleic acid.

Disclosed herein are methods and systems for determining binding energies (enthalpy, entropy and ΔG) between a first candidate molecule and a second candidate molecule using a nucleic acid scaffold. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) determining a reference elongation length of the nucleic acid scaffold in response to a force in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule can be in close proximity or in contact within each other; (f) determining an elongation length of the screening nucleic acid scaffold in response to the same force used in the absence of the first candidate molecule and the second candidate molecule by repeating (d); (g) calculating a differential value, wherein the differential value is the difference between the elongation length of the screening nucleic acid scaffold and the reference elongation length at the force applied. A non-zero differential value indicates presence of the binding interaction between the first candidate molecule and the second candidate molecule at the force applied. Conversely, a differential value of zero indicates absence of the binding interaction between the first candidate molecule and the second candidate molecule at the force applied. In some embodiments, the reference elongation length is determined in real time.

Also, disclosed herein are methods and systems for determining binding energies (enthalpy, entropy and ΔG) between a first candidate molecule, a second candidate molecule and a third candidate molecule using a nucleic acid scaffold. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) determining a reference elongation length of the nucleic acid scaffold in response to a force in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact within each other; (f) determining a force (e.g., a force in a range of from 0.01 pN to 10 pN) required to achieve the same elongation length for the screening nucleic acid scaffold as the reference elongation length by repeating (d); (g) contacting a third candidate molecule in solution to the screening nucleic acid scaffold; (h) determining an elongation length of the screening nucleic acid scaffold in the presence of the third candidate molecule in response to the same force applied in (f) by repeating (d); (i) Disclosed herein are methods and systems for determining energies associated with binding interactions between a first candidate molecule and a second candidate molecule using a nucleic acid scaffold. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) determining a first force required for elongating the nucleic acid scaffold to a reference elongation length in the absence of the first candidate molecule and the second candidate molecule, wherein the reference elongation length is a length of the nucleic acid scaffold that does not increase in response to increase in amount of force applied to the nucleic acid scaffold, and wherein the first force is a minimum amount of force required to elongate the nucleic acid scaffold to the reference elongation length; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule can be in close proximity or in contact within each other; (f) determining a force required to achieve the same elongation length for the screening nucleic acid scaffold as the reference elongation length by repeating (d); (g) calculating a force differential value, wherein the force differential value is the difference between the force required for elongating the nucleic acid scaffold to the reference elongation length and the force required for elongating the screening nucleic acid scaffold to the reference elongation length. A non-zero force differential value provides binding energy associated with binding interaction between the first candidate molecule and the second candidate molecule. Conversely, a differential value of zero indicates absence of the binding interaction between the first candidate molecule and the second candidate molecule at the force applied. In some embodiments, the reference elongation length is determined in real time.

Also, disclosed herein are methods and systems for determining binding energies associated with binding interactions between a first candidate molecule, a second candidate molecule and a third candidate molecule using a nucleic acid scaffold. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) determining a first force required for elongating the nucleic acid scaffold to a reference elongation length in the absence of the first candidate molecule and the second candidate molecule, wherein the reference elongation length is a length of the nucleic acid scaffold that does not increase in response to increase in amount of force applied to the nucleic acid scaffold, and wherein the first force is a minimum amount of force required to elongate the nucleic acid scaffold to the reference elongation length; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact within each other; (f) determining a second force (e.g., a force in a range of from 0.01 pN to 10 pN, from 0.05 pN to 10 pN, or from 0.1 pN to 10 pN) required to achieve the same elongation length for the screening nucleic acid scaffold as the reference elongation length by repeating (d); (g) contacting a third candidate molecule in solution to the screening nucleic acid scaffold; (h) determining a third force (e.g., a force in a range of from 0.01 pN to 10 pN, from 0.05 pN to 10 pN, or from 0.1 pN to 10 pN) required to achieve the same elongation length for the screening nucleic acid scaffold as the reference elongation length in the presence of the third candidate molecule by repeating (d); (i) calculating a force differential value, wherein the force differential value is the difference between the first force and the second force, the second force and the third force, or the first force and the third force. A non-zero force differential value between the first force and the second force provides the binding energy associated with the binding interaction between the first candidate molecule and the second candidate molecule. Conversely, a force differential value of zero between the first force and the second force indicates absence of the binding interaction between the first candidate and the second candidate. Alternatively, a non-zero force differential value between the second force and the third force provides the binding energy associated with the binding interaction between the third candidate molecule, and the first candidate molecule and the second candidate molecule. Conversely, a force differential value of zero between the second force and the third force indicates absence of the binding interaction between: (A) the third candidate molecule and the first candidate molecule, (B) the third candidate molecule and the second candidate molecule, or (C) the third candidate molecule, and the first candidate molecule and the second candidate molecule.

In some embodiments, the reference elongation length is determined by: (a) applying a force (e.g., a force in a range of from 0.01 pN to 10 pN, from 0.05 pN to 10 pN, or from 0.1 pN to 10 pN) to the bead attached to the nucleic acid scaffold via the force application mechanism along the axis perpendicular to the bottom surface of the device, wherein the nucleic acid scaffold is configured to unfold in response to the applied force, thereby resulting in a change in position of the bead attached to the nucleic acid scaffold along the axis, and (b) measuring the change in position of the bead along the axis via the sensor, thereby determining the reference elongation length of the nucleic acid scaffold.

In some embodiments, the elongation length of the screening nucleic acid scaffold is determined by: (a) applying a force to the bead attached to the nucleic acid scaffold attached to the first candidate molecule and the second candidate molecule via the force application mechanism along the axis perpendicular to the bottom surface of the device, wherein the nucleic acid scaffold is configured to unfold in response to the applied force, thereby resulting in a change in position of the bead attached to the nucleic acid scaffold along the axis, and (b) measuring the change in position of the bead along the axis via the sensor, thereby determining the reference elongation length of the nucleic acid scaffold.

In some embodiments, a force applied to determine one or more of a reference elongation length, an elongation length of the screening nucleic acid scaffold comprise at least 0.01 pN, at least 0.05 pN, at least 0.1 pN, at least 5 pN, at least 10 pN, at least 20 pN, at least 35 pN, at least 70 pN or at least 95 pN. In some embodiments, the force is not more than 100 pN. In some embodiments, the force is in a range of from 0.01 pN to 10 pN, from 0.01 pN to 8 pN, from 0.01 pN to 5 pN, from 0.01 pN to 3 pN, from 0.05 pN to 10 pN, from 0.05 pN to 8 pN, from 0.05 pN to 5 pN, from 0.05 pN to 3 pN, from 0.1 pN to 10 pN, from 0.1 pN to 8 pN, from 0.1 pN to 5 pN, from 0.1 pN to 3 pN, from 2 pN to 10 pN, from 2 pN to 7 pN, from 2 pN to 5 pN or from 5 pN to 10 pN. In some embodiments, the force is less than 10 pN. In some embodiments, the force is 10 pN. In some embodiments, the force is less than 5 pN. In some embodiments, the force is 5 pN. In some embodiments, the force is more than 0.1 pN. In some embodiments, the force is more than 0.01 pN.

FIG. 13 shows a graphical representation of methods for determining binding interactions as well as energies associated with binding interactions between two candidate molecules using a system described herein. As shown in FIG. 13, A, the system comprising a screening nucleic acid scaffold comprising a nucleic acid scaffold and two candidate molecules anchored to the nucleic acid scaffold by two glue sequences, wherein two candidate molecules interact with each other. As shown in FIG. 13, B, a presence of a binding interaction between the two candidate molecules at an applied force, the nucleic acid scaffold does not stretch as much as a reference elongation length of the nucleic acid scaffold. When the interaction is broken between the two candidate molecules at an applied force as shown in FIG. 13, C, the nucleic acid scaffold stretches to a length similar to the reference elongation length observed before the interaction (not shown). By cycling the force between low force (e.g., about 0.1 pN or less, or about 0.01 pN) to high force (e.g., between 0.1 pN to 25 pN depending on the strength of the interaction), it is possible to monitor the binding interaction as well as extract the binding energy of the interaction between the two candidate molecules due to a reduced displacement in Z. The binding interaction can be broken by applying a force in a direction that is perpendicular to a surface of a device (e.g., Z direction). Once the interaction is broken, the screening nucleic acid scaffold fully stretches and causes a jump in the Z axis corresponding to the size of the nucleic acid scaffold. In some embodiments, a presence of binding interactions between the two candidate molecules corresponds to low Brownian noise relative to the high Brownian noise observed in absence of the binding interactions between the two candidate molecules.

Also described herein are methods for determining binding interactions as well as energies associated with binding interactions between two candidate molecules using a system described herein, wherein at least one molecule binding sequence of a hairpin nucleic acid of the system comprises a small hairpin nucleic acid (FIG. 20A). Exemplary first molecule binding sequences of a hairpin nucleic acid described herein include SEQ ID NOS: 50 and 51 (FIGS. 20B and 20C). Exemplary second molecule binding sequence of a hairpin nucleic acid described herein includes SEQ ID NO: 52. FIG. 20D shows raw traces of binding of glue sequences linked to candidate molecules to molecule binding sequences of a hairpin nucleic acid described herein. As shown in FIGS. 20A and 20D, binding of one glue sequence to the corresponding complementary molecule binding sequence of a hairpin nucleic acid results in one jump disappearing from the scaffold molecule, and binding of both glue sequences to the corresponding complementary molecule binding sequences of the hairpin nucleic acid results in the two jumps being lost. An analysis of FIGS. 20A and 20D also indicates that upon increasing the force (e.g., 5 pN, 10 pN, or more), the holiday junction is resolved, and the hairpin is converted into a dsDNA. This result in the disappearance of the small jump due to the presence of the small hairpin sequence.

In some embodiments, methods described herein advantageously determines binding interactions between two or more candidate molecules at applied force in a range of from 0.01 pN to 10 pN, from 0.05 pN to 10 pN or from 0.1 pN to 10 pN. In some embodiments, the force is a magnetic force, and the bead is a magnetic bead.

### Methods for Determining Binding Kinetics

Disclosed herein are methods and systems for use in determining binding kinetics between at least two candidate molecules. In some embodiments, at least two candidate molecules are selected proteins of interest described herein, nucleic acids of interest described herein, and small molecules of interest described herein. In some embodiments, at least two candidate molecules are proteins of interest. In some embodiments, at least two candidate molecules are small molecules of interest. In some embodiments, at least two candidate molecules are nucleic acids of interest. In some embodiments, at least two candidate molecules are selected from proteins of interest and nucleic acids of interest. In some embodiments, at least two candidate molecules are selected from proteins of interest and small molecules of interest. In some embodiments, at least two candidate molecules are selected from nucleic acids of interest and small molecules of interest.

Disclosed herein are methods and systems for determining binding kinetics for candidate molecules in real time. In some embodiments, the methods and systems are capable of detecting binding interactions between candidate molecules in real time and analysing molecular interactions for determining binding kinetics and energetics. In some embodiments, the methods and systems comprise determining binding kinetics and energetics a single molecular level. In some embodiments, the methods and systems comprise determining at least two binding kinetics or energetics in parallel between candidate molecules at a single molecular level. In some embodiments, the methods and systems comprise use of magnetic tweezers for determining binding kinetics.

Disclosed herein are methods and systems for determining binding kinetics and energetics of a binding interaction between a first candidate molecule and a second candidate molecule. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) determining a reference elongation length of the nucleic acid scaffold in response to a force in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact within each other; (f) determining an elongation length of the screening nucleic acid scaffold in response to the same force used in the absence of the first candidate molecule and the second candidate molecule by repeating (d); (g) calculating a differential value, wherein the differential value is the difference between the elongation length of the screening nucleic acid scaffold and the reference elongation length at the force applied; (h) removing the force applied by the force application mechanism to the bead after (g), thereby resulting in relaxation of the screening nucleic acid scaffold; and (i) repeating (f)-(h) and calculating the difference between the elongation length of the screening nucleic acid scaffold attached to the first candidate molecule and the second candidate molecule and the reference elongation length as a function of time. In some embodiments, the methods described herein further comprise: (i) determining a Kₒₙ for the binding of the second molecule to the first candidate molecule, wherein the Kₒₙ is calculated based on the number of cycles of repeating (f)-(h) that result in the differential between the elongation length of the screening nucleic acid scaffold and the reference elongation length. In some embodiments, the Kₒₙ of the interaction is determined based on the number of cycles that are observed to have a blockage. In some embodiments, the methods described herein further comprise: (h) determining a K_{off} for the binding of the second molecule to the first candidate molecule, wherein the K_{off} is calculated based on the length of time that the differential between the elongation length of the screening nucleic acid scaffold and the reference elongation length is present during each cycle of repeating (f)-(h). In some embodiments, the K_{off} of the interaction is determined based on a duration for which a blockage lasts. In some embodiments, a first molecule binding sequence of the nucleic acid scaffolds described herein comprises a small hairpin nucleic acid, wherein a first glue sequence as described herein comprises a polynucleotide that is complementary to the small hairpin nucleic acid. In some embodiments, a second molecule binding sequence of the nucleic acid scaffolds described herein comprises a small hairpin nucleic acid, wherein a second glue sequence as described herein comprises a polynucleotide that is complementary to the small hairpin nucleic acid.

Also, disclosed herein are methods and systems for determining binding kinetics of a binding interaction between a first candidate molecule, a second candidate molecule and a third candidate molecule using a nucleic acid scaffold. In some embodiments, the methods comprise: (a) providing a nucleic acid scaffold as described herein; (b) providing a device as described herein; (c) providing a first candidate molecule as described herein linked to a first glue sequence, and a second candidate molecule as described herein linked to a second glue sequence; (d) determining a reference elongation length of the nucleic acid scaffold in response to a force in the absence of the first candidate molecule and the second candidate molecule; (e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in contact with each other; (f) determining a force (e.g., a force in a range of from 0.01 pN to 10 pN, from 0.05 pN to 10 pN, or from 0.1 pN to 10 pN) required to achieve the same elongation length for the screening nucleic acid scaffold as the reference elongation length by repeating (d); (g) contacting a third candidate molecule to the screening nucleic acid scaffold; (h) determining an elongation length of the screening nucleic acid scaffold in the presence of the third candidate molecule in response to the same force applied in (f) by repeating (d); (i) calculating a differential value, wherein the differential value is the difference between the elongation length of the screening nucleic acid scaffold and the reference elongation length at the force applied. In some embodiments, the methods further comprise: (j) removing the force applied by the force application mechanism to the bead after (i), thereby resulting in relaxation of the test screening nucleic acid scaffold; and (k) repeating (e)-(j) and calculating the difference between the elongation length of the screening nucleic acid scaffold in the presence and absence of the third candidate molecule as a function of time. In some embodiments, the methods further comprise: (j) determining a Kₒₙ for the binding of the third candidate molecule to the first and second molecules, wherein the Kₒₙ is calculated based on the number of cycles of repeating (h)-(j) that result in the differential between the elongation length of the screening nucleic acid scaffold in the presence and absence of the third candidate molecule. In some embodiments, the methods further comprise: (j) determining a K_{off} for the binding of the third candidate molecule to the first and second molecules, wherein the K_{off} is calculated based on the average length of time that the differential between the elongation length of the screening nucleic acid scaffold in the presence and absence of the third candidate molecule is present during each cycle of repeating (h)-(j). In some embodiments, a first molecule binding sequence of the nucleic acid scaffolds described herein comprises a small hairpin nucleic acid, wherein a first glue sequence as described herein comprises a polynucleotide that is complementary to the small hairpin nucleic acid. In some embodiments, a second molecule binding sequence of the nucleic acid scaffolds described herein comprises a small hairpin nucleic acid, wherein a second glue sequence as described herein comprises a polynucleotide that is complementary to the small hairpin nucleic acid.

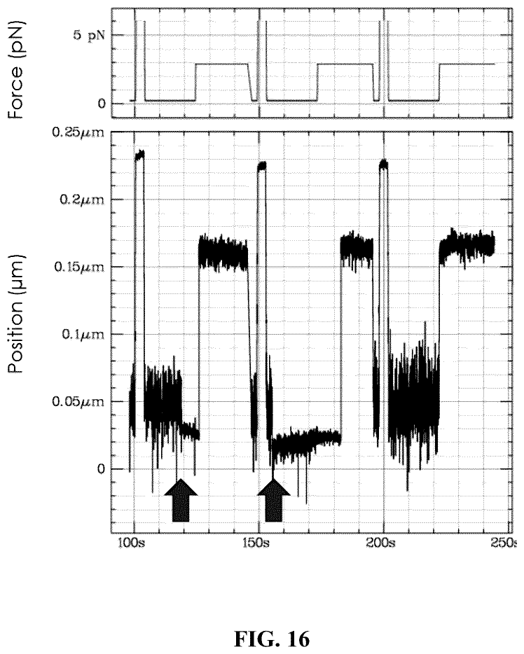

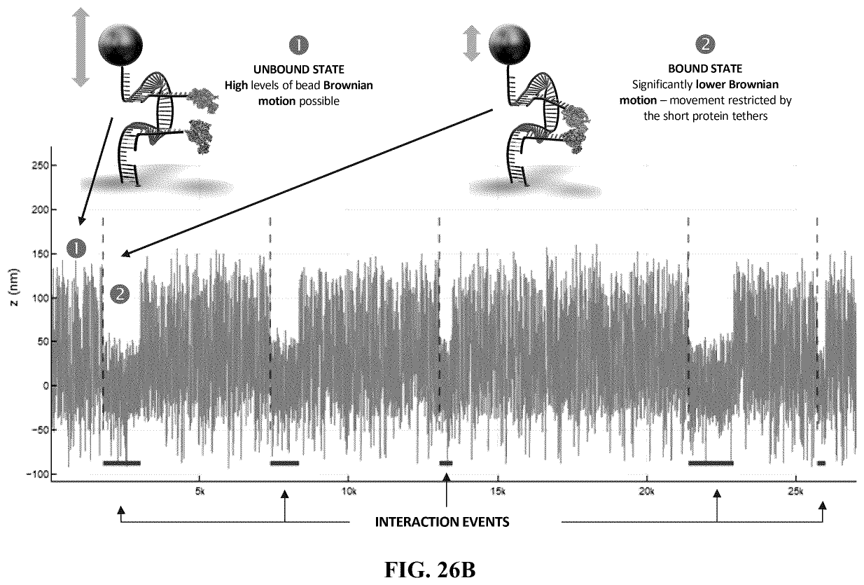

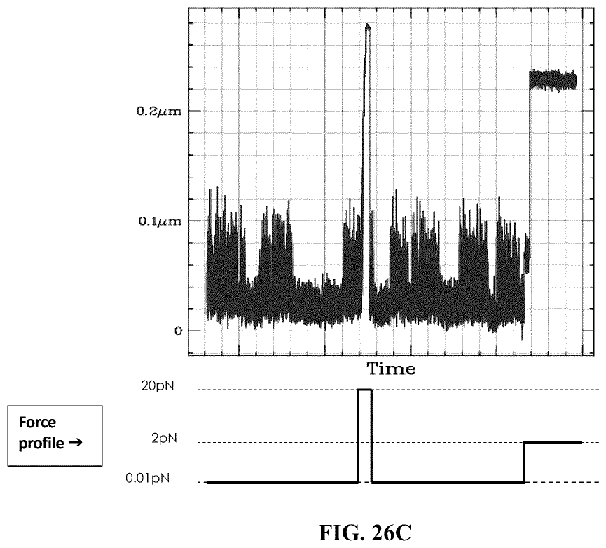

FIGS. 15A-15B show exemplary traces for determining binding kinetics between two candidate molecules. As shown in FIGS. 15A-15B, a presence of binding interactions between the two candidate molecules corresponds to low Brownian noise relative to the high Brownian noise observed in absence of the binding interactions between the two candidate molecules. Likewise, FIG. 16 shows exemplary changes in Brownian movement of a bead anchoring a nucleic acid scaffold in the presence and absence of two candidate molecules, ACE2 and SARS-COV2 RBD. FIG. 26A shows an exemplary trace for determining binding kinetics between two candidate molecules, ACE2 and RBD. As shown in FIG. 26B, a presence of binding interactions between ACE2 and RBD corresponds to low Brownian noise relative to the high Brownian noise observed in absence of the binding interactions. In some embodiments, the force is kept constant throughout the experiment. Accordingly, the constant applied force can be between almost 0 pN to high force (e.g., 25 pN depending on the strength of the interaction). In some embodiments, the force is maintained for at least 5 minutes, or at least 10 minutes, or at least 20 minutes, or at least 40 minutes, or at least 60 minutes, or at least 120 minutes. In some embodiments, the methods further comprise determining a Kₒₙ for the binding of the first and second molecules, wherein the Kₒₙ is calculated based on the number of times the low Brownian noise is observed over the total duration of the experiment. In some embodiments, the methods further comprise determining a K_{off} for the binding of the first and second candidate molecules, wherein the K_{off} is calculated based on the average length of time that the low Brownian noise is observed.

### Methods of Screening

Disclosed herein are methods and systems for use in screening for candidate molecules. In some embodiments, the candidate molecules are selected proteins of interest described herein, nucleic acids of interest described herein, small molecules of interest described herein, or combinations thereof.

Disclosed herein are methods and systems for screening candidate molecules in real time. In some embodiments, the methods and systems are capable of determining binding strength interactions between candidate molecules in real time, analysing molecular interactions for each of the candidate molecules, determining binding kinetics for each of the candidate molecules, determining the energetic of the interaction and, therefore, comparing the candidate molecules relative to each other for screening. In some embodiments, the methods and systems comprise use of magnetic tweezers for screening candidate molecules.

Disclosed herein are methods and systems for determining binding interactions between a first candidate molecule and a plurality of second candidate molecules using nucleic acid scaffolds described herein. In some embodiments, the methods comprise: (a) providing a device as described herein, wherein the device comprise a plurality of nucleic acid scaffolds each positioned along an axis perpendicular to the bottom surface of the chamber of the device, and each of the plurality of nucleic acid scaffolds are linked to a bead at one end and a feature of a bottom surface of the device at the other end; (b) determining a reference elongation length of each of the nucleic acid scaffold among the plurality of nucleic acid scaffolds in the absence of the first candidate molecule and the plurality of second candidate molecules by: (i) applying a force (e.g., 0.01 pN to 10 pN, 0.05 pN to 10 pN, or 0.1 pN to 10 pN) to the bead attached to each of the plurality of nucleic acid scaffolds via the force application mechanism along the axis perpendicular to the bottom surface of the device, wherein each of the plurality of nucleic acid scaffolds is configured to unfold in response to the force applied, thereby resulting in a change in position of the bead attached to each of the plurality of nucleic acid scaffolds along the axis, and (ii) measuring the change in position of the bead along the axis via the sensor, thereby determining reference elongation lengths for each of the plurality of nucleic acid scaffolds in the absence of the first candidate molecule and the plurality of second candidate molecules; (c) contacting the plurality of nucleic acid scaffolds with the first candidate molecule, thereby anchoring the first candidate molecule to each of the plurality of nucleic acid scaffolds; (d) contacting the plurality of nucleic acid scaffolds with the plurality of second candidate molecules, thereby anchoring one of the second candidate molecule among the plurality of candidate molecules to each of the plurality of nucleic acid scaffolds, thereby forming a plurality of screening nucleic acid scaffolds, wherein the first candidate molecule and the second candidate molecule of each of the plurality of screening nucleic acid scaffolds are positioned such that the first candidate molecule and the second candidate molecule are in contact with each other; (e) determining elongation lengths for each of the plurality of screening nucleic acid scaffolds in response to the force (e.g., 0.01 pN to 10 pN, 0.05 pN to 10 pN, or 0.1 pN to 10 pN) by repeating (d); and (f) calculating the difference between the reference elongation length and the elongation length of each screening nucleic acid scaffold among the plurality of screening nucleic acid scaffolds. A non-zero differential value indicates presence of the binding interaction between the first candidate molecule and the second candidate molecule at the force applied. Conversely, a differential value of zero indicates absence of the binding interaction between the first candidate molecule and the second candidate molecule at the force applied. In some embodiments, the reference elongation length is determined in real time.

Also, disclosed herein are methods and systems for determining binding interactions between a first candidate molecule, a second candidate molecule and a plurality of third candidate molecules using nucleic acid scaffolds described herein. In some embodiments, the methods comprise: (a) providing a device as described herein, wherein the device comprise a plurality of nucleic acid scaffolds each positioned along an axis perpendicular to the bottom surface of the chamber of the device, and each of the plurality of nucleic acid scaffolds are linked to a bead at one end and a feature of a bottom surface of the device at the other end; (b) determining a reference elongation length of each of the nucleic acid scaffold among the plurality of nucleic acid scaffolds in the absence of the first candidate molecule and the second candidate molecules by: (i) applying a force (e.g., 0.01 pN to 10 pN, 0.05 pN to 10 pN, or 0.1 pN to 10 pN) to the bead attached to each of the plurality of nucleic acid scaffolds via the force application mechanism along the axis perpendicular to the bottom surface of the device, wherein each of the plurality of nucleic acid scaffolds is configured to unfold in response to the force applied, thereby resulting in a change in position of the bead attached to each of the plurality of nucleic acid scaffolds along the axis, and (ii) measuring the change in position of the bead along the axis via the sensor, thereby determining reference elongation lengths for each of the plurality of nucleic acid scaffolds in the absence of the first candidate molecule and the plurality of second candidate molecules; (c) contacting the plurality of nucleic acid scaffolds with the first candidate molecule, thereby anchoring the first candidate molecule to each of the plurality of nucleic acid scaffolds; (d) contacting the plurality of nucleic acid scaffolds with the plurality of second candidate molecules, thereby anchoring the second candidate molecule to each of the plurality of nucleic acid scaffolds, thereby forming a plurality of screening nucleic acid scaffolds, wherein the first candidate molecule and the second candidate molecule of each of the plurality of screening nucleic acid scaffolds are positioned such that the first candidate molecule and the second candidate molecule are in contact with each other; (e) determining a force (e.g., 0.01 pN to 10 pN, 0.05 pN to 10 pN, or 0.1 pN to 10 pN) required to achieve the same elongation length for the screening nucleic acid scaffold as the reference elongation length by repeating (d); (f) contacting the plurality of third candidate molecules to the plurality of screening nucleic acid scaffolds; and (g) determining elongation lengths for each of the screening nucleic acid scaffolds in the presence and absence of at least one of the plurality of third candidate molecules in response to the same force applied in (e) by repeating (b); and (g) calculating a differential value, wherein the differential value is the difference between the elongation length of the screening nucleic acid scaffold and the reference elongation length. If there is no measurable difference between the elongation length of the screening nucleic acid scaffold and the reference elongation length (a “zero differential”), then there is no binding interaction detected. If there is a measurable difference between the elongation length of the screening nucleic acid scaffold and the reference elongation length (a “non-zero differential”), this indicates presence of a binding interaction between the third candidate molecule, and the first candidate molecule and the second candidate molecule at the force applied. Conversely, a differential value of zero indicates absence of the binding interaction between: (A) the third candidate molecule and the first candidate molecule, (B) the third candidate molecule and the second candidate molecule, or (C) the third candidate molecule, and the first candidate molecule and the second candidate molecule, at the force applied.

In some embodiments, each of the plurality of nucleic acid scaffolds of the methods described herein comprise barcode sequence. In some embodiments, at least two of the plurality of nucleic acid scaffolds comprise non-identical barcode sequences relative to each other. In some embodiments, at least two of the plurality of nucleic acid scaffolds comprise barcode sequences that are located at non-identical positions relative to each other. In some embodiments, the methods described herein further comprise determining the identity of the hairpin nucleic acid based on the barcode. In some embodiments, the identity of the hairpin nucleic acid is determined by detecting the position of the one or more modified nucleotides in the barcode. In some embodiments, the methods described herein further comprise making nucleic acid scaffolds using any of the methods described herein. For example, in some embodiments, the methods described herein further comprise: (a) providing a plurality of hairpin nucleic acids, wherein each of the plurality of hairpin nucleic acids is a contiguous polynucleotide sequence, wherein each of the plurality of hairpin nucleic acids comprises a barcode, wherein at least two of the plurality of hairpin nucleic acids comprises a different barcode; (b) attaching a bead to a first end of each of the plurality of hairpin nucleic acids; and (c) attaching a second end of each of the plurality of hairpin nucleic acids to a bottom surface of a device; (d) decoding the barcode for each of the plurality of hairpin nucleic acid; (e) hybridizing a first spacer polynucleotide to each of a first spacer binding sequence of the plurality of hairpin nucleic acids, wherein the first spacer polynucleotide comprises a polynucleotide sequence that is complementary to the first spacer binding sequence; (f) hybridizing a second spacer polynucleotide to each of a second spacer binding sequence of the plurality of hairpin nucleic acids, wherein the second spacer polynucleotide comprises a polynucleotide sequence that is complementary to the second spacer binding sequence. In some embodiments, a first spacer polynucleotide and a second spacer polynucleotide are provided together, wherein the first spacer polynucleotide comprises a polynucleotide sequence that is complementary to at least a portion of a first spacer binding sequence of the plurality of hairpin nucleic acids, wherein the second spacer polynucleotide comprises a polynucleotide sequence that is complementary to at least a portion of a second spacer binding sequence of the plurality of hairpin nucleic acids, and wherein the first spacer polynucleotide hybridizes to the first spacer binding sequence and the second spacer polynucleotide hybridizes to the second spacer binding sequence.

In some embodiments, methods described herein further comprise testing the activity of the identified candidate molecule, as a medicament to prevent or treat a disease directly or indirectly related to the candidate molecule.

In some embodiments, attaching a plurality of hairpin nucleic acids to a plurality of features comprises covalently attaching a second adapter 107 of each of the plurality of hairpin nucleic acids to one of the plurality of features of a bottom surface of a device. In some embodiments, attaching a plurality of hairpin nucleic acids to a plurality of features comprises non-covalently attaching a second adapter 107 of each of the plurality of hairpin nucleic acids to one of the plurality of features of a bottom surface of a device. In some embodiments, attaching a plurality of hairpin nucleic acids to a plurality of features comprises attaching a second adapter 107 of each of the plurality of hairpin nucleic acids to one or the plurality of features of a bottom surface of a device by hybridization, wherein the second adapter 107 comprises a polynucleotide sequence that is complementary to a polynucleotide sequence of the feature. In some embodiments, each feature of a chamber comprises a single nucleic acid scaffold among the plurality of nucleic acid scaffolds.

In some embodiments, a change in the elongation length for each of the plurality of screening nucleic acid is carried out in real time by exerting a force, advantageously a series of constant force or a series of increasing forces onto the nucleic acid molecule using a magnet and measuring a movement of a bead linked to the nucleic acid.

### EXEMPLARY EMBODIMENTS

Embodiment 1: A nucleic acid scaffold for determining a binding interaction between a first candidate molecule and a second candidate molecule, wherein the nucleic acid scaffold comprises:
(a) a contiguous polynucleotide sequence comprising:
   (i) a first end that is attached to a bead, wherein the first end comprises a first molecule binding sequence,
   (ii) a second end that is attached to a bottom surface of a device, wherein the second end comprises a second molecule binding sequence, and
   (iii) an intermediate portion between the first molecule binding sequence of the first end and the second molecule binding sequence of the second end, wherein the intermediate portion comprises:
      (I) a first pin forming sequence comprising a barcode,
      (II) a second pin forming sequence that is complementary to the first pin forming sequence, wherein the first pin forming sequence is hybridized to the second pin forming sequence, and
      (III) a loop linking the first pin forming sequence and the second pin forming sequence;
(b) a first spacer polynucleotide that has a polynucleotide sequence that is complementary to the first pin forming sequence; and
(c) a second spacer polynucleotide that has a polynucleotide sequence that is complementary to the second pin forming sequence,
wherein the first spacer polynucleotide and the second spacer polynucleotide are hybridized to the intermediate portion, and
wherein the nucleic acid scaffold requires a force of 0.01 pN to 10 pN, 0.05 pN to 10 pN, or 0.1 pN to 10 pN applied to the bead along an axis perpendicular to the bottom surface of the device to unfold the contiguous polynucleotide sequence along the axis to form the nucleic acid scaffold.
Embodiment 2. The nucleic acid scaffold of Embodiment 1, wherein the barcode sequence comprises one or more modified nucleotides.
Embodiment 3. The nucleic acid scaffold of any one of Embodiment 1-2, wherein the intermediate portion further comprises a first junction sequence and a second junction sequence, wherein the first junction sequence is located between the first molecule binding sequence and the first pin forming sequence, and wherein the second junction sequence is located between the second molecule binding sequence and the second pin forming sequence, and wherein the first junction sequence and the second junction sequence are not complementary to each other.
Embodiment 4. The nucleic acid scaffold of any one of Embodiments 1-3, wherein the first spacer polynucleotide and the second spacer polynucleotide are linked to each other by a spacer loop sequence, wherein the spacer loop sequence is hybridized to the loop.
Embodiment 5. The nucleic acid scaffold of any one of Embodiments 1-4, wherein each, the first molecule binding sequence and the second molecule binding sequence, independently comprises a small hairpin nucleic acid having a size in a range of from 5 bases to 100 bases.
Embodiment 6. A screening nucleic acid scaffold comprising:
(a) a nucleic acid scaffold of any one of Embodiments 1-5;
(b) a first candidate molecule linked to a first glue sequence, wherein the first glue sequence is hybridized to the first molecule binding sequence; and
(c) a second candidate molecule linked to a second glue sequence, wherein the second glue sequence is hybridized to the second molecule binding sequence.
Embodiment 7. A device that comprises:
(a) a chamber disposed within the device, wherein the chamber comprises a bottom surface;
(b) a force application mechanism for stretching the contiguous polynucleotide sequence along the axis to form the nucleic acid scaffold; and
(c) the nucleic acid scaffold of any one of Embodiments 1-5 or the screening nucleic acid scaffold of Embodiment 6.
Embodiment 8. A method of determining a binding interaction between a first candidate molecule and a second candidate molecule, the method comprising:
(a) providing a nucleic acid scaffold of any one of Embodiments 1-5, wherein the nucleic acid scaffold is positioned along an axis perpendicular to a bottom surface of the chamber of the device;
(b) providing a device that comprises:
   (i) a chamber disposed within the device, wherein the chamber comprises a bottom surface capable of binding the anchoring sequence of any molecule of the embodiment 1-5, and
   (ii) a force application mechanism for stretching the contiguous polynucleotide sequence along the axis to form the nucleic acid scaffold;
(c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence;
(d) determining a reference elongation length of the nucleic acid scaffold in response to a force in the absence of the first candidate molecule and the second candidate molecule in real time by:
   (i) applying a force of 0.01 to 50 pN to the bead attached to the nucleic acid scaffold via the force application mechanism along the axis perpendicular to the bottom surface of the device, wherein the nucleic acid scaffold is configured to unfold in response to the force applied, thereby resulting in a change in position of the bead attached to the nucleic acid scaffold along the axis, and
   (ii) measuring the change in position of the bead along the axis via a sensor, thereby determining the reference elongation length of the nucleic acid scaffold in the absence of the first candidate molecule and the second candidate molecule;
(e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity with each other;
(f) determining an elongation length of the screening nucleic acid scaffold in response to the same force used in the absence of the first candidate molecule and the second candidate molecule by repeating (d); and
(g) calculating a differential value, wherein the differential value is a difference between the elongation length of the screening nucleic acid scaffold and the reference elongation length at the force applied,
whereby a non-zero differential value indicates presence of the binding interaction between the first candidate molecule and the second molecule at the force applied, and whereby differential value of zero indicates absence of the binding interaction between the first candidate molecule and the second candidate molecule at the force applied.
Embodiment 9. The method of Embodiment 8, further comprising determining binding kinetics of the binding interaction between the first candidate molecule and the second candidate molecule by: (h) removing the force applied by the force application mechanism to the bead after (g), thereby resulting in relaxation of the screening nucleic acid scaffold; and (i) repeating (f)-(h) and calculating a difference between the elongation length of the screening nucleic acid scaffold and the reference elongation length as a function of time.
Embodiment 10. The method of Embodiment 9, further comprising: (i) determining a Kₒₙ for the binding of the second molecule to the first candidate molecule, wherein the Kₒₙ is calculated based on the number of cycles of repeating (f)-(h) that result in the differential between the elongation length of the screening nucleic acid scaffold and the reference elongation length.
Embodiment 11. The method of Embodiment 9, further comprising: (h) determining a K_{off} at the specified force for the binding of the second molecule to the first candidate molecule, wherein the K_{off} is calculated based on the length of time that the differential between the elongation length of the screening nucleic acid scaffold and the reference elongation length is present during each cycle of repeating (f)-(h) at the defined force.
Embodiment 12. A method of determining a binding interaction between a first candidate molecule, a second candidate molecule and a third candidate molecule, the method comprising:
(a) providing a nucleic acid scaffold of any one of Embodiments 1-5, wherein the nucleic acid scaffold is positioned along an axis perpendicular to a bottom surface of the chamber of the device;
(b) providing a device that comprises:
   (i) a chamber disposed within the device, wherein the chamber comprises a bottom surface capable of binding the anchoring sequence 107 of any molecule of the embodiment 1-5, and,
   (ii) a force application mechanism for stretching the contiguous polynucleotide sequence along the axis to form the nucleic acid scaffold;
(c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence;
(d) determining a reference elongation length of the nucleic acid scaffold in the absence of the first candidate molecule and the second candidate molecule by:
   (i) applying a force of 0.01 pN to 50 pN to the bead attached to the nucleic acid scaffold via the force application mechanism along the axis perpendicular to the bottom surface of the chamber of the device, wherein the nucleic acid scaffold is configured to unfold in response to the force applied, thereby resulting in a change in position of the bead attached to the nucleic acid scaffold along the axis, and
   (ii) measuring the change in position of the bead along the axis via a sensor, thereby determining the reference elongation length of the nucleic acid scaffold in the absence of the first candidate molecule and the second candidate molecule;
(e) contacting the nucleic acid scaffold with the first candidate molecule and the second candidate molecule, and, thereby, forming a screening nucleic acid scaffold, wherein the first candidate molecule and the second candidate molecule are positioned along the screening nucleic acid scaffold such that the first candidate molecule and second candidate molecule are in close proximity with each other;
(f) determining a force required to achieve the same elongation length for the screening nucleic acid scaffold as the reference elongation length by repeating (d), wherein the force required is in a range of from 0.01 pN to 50 pN;
(g) contacting the third candidate molecule to the screening nucleic acid scaffold; and
(h) determining an elongation length of the screening nucleic acid scaffold in the presence of the third candidate molecule and in response to the same force applied in (f) by repeating (d); and
(i) calculating a differential value, wherein the differential value is a difference between the elongation length of the screening nucleic acid scaffold in the presence and absence of the third candidate molecule,
whereby a non-zero differential value indicates presence of a binding interaction between the third candidate molecule, and the first candidate molecule and the second candidate molecule at the force applied, and whereby a differential value of zero indicates absence of the binding interaction between:
   (A) the third candidate molecule and the first candidate molecule,
   (B) the third candidate molecule and the second candidate molecule, or
   (C) the third candidate molecule, and the first candidate molecule and the second candidate molecule, at the force applied.
Embodiment 13. The method of Embodiment 12, further comprising determining binding kinetics of the binding interaction between the first candidate molecule, the second candidate molecule and the third candidate molecule by: (j) removing the force applied by the force application mechanism to the bead after (i), thereby resulting in relaxation of the test screening nucleic acid scaffold; and (k) repeating (e)-(j) and calculating a difference between the elongation length of the screening nucleic acid scaffold in the presence and absence of the third candidate molecule as a function of time.
Embodiment 14. The method of Embodiment 12, further comprising: (j) determining a Kₒₙ for the binding of the third candidate molecule to the first and second molecules,
wherein the Kₒₙ is calculated based on the number of cycles of repeating (h)-(j) that result in the differential between the elongation length of the screening nucleic acid scaffold in the presence and absence of the third candidate molecule.
Embodiment 15. The method of Embodiment 12, further comprising: (j) determining a K_{off} at the specified force for the binding of the third candidate molecule to the first and second candidate molecules, wherein the K_{off} is calculated based on the length of time that the differential between the elongation length of the screening nucleic acid scaffold in the presence and absence of the third candidate molecule is present during each cycle of repeating (h)-(j).
Embodiment 16. A method of screening binding interactions between a first candidate molecule and a plurality of second candidate molecules, the method comprising:
(a) providing a device that comprises:
   (i) a chamber disposed within the device, wherein the chamber comprises a bottom surface,
   (ii) a force application mechanism, and
   (iii) a plurality of nucleic acid scaffolds positioned along an axis perpendicular to the bottom surface of the chamber of the device, wherein each nucleic acid scaffold among the plurality of nucleic acid scaffolds comprises the nucleic acid scaffold of any one of Embodiments 1-5, wherein each of the plurality of nucleic acid scaffolds are linked to a bead at one end and a feature of the bottom surface of the chamber of the device at the other end;
(b) determining a reference elongation length of each of the nucleic acid scaffold among the plurality of nucleic acid scaffolds in the absence of the first candidate molecule and the plurality of second candidate molecules by:
   (i) applying a force of 0.01 pN to 50 pN to the bead attached to each of the plurality of nucleic acid scaffolds via the force application mechanism along the axis perpendicular to the bottom surface of the chamber of the device, wherein each of the plurality of nucleic acid scaffolds is configured to unfold in response to the force applied, thereby resulting in a change in position of the bead attached to each of the plurality of nucleic acid scaffolds along the axis, and
   (ii) measuring the change in position of the bead along the axis via a sensor, thereby determining reference elongation lengths for each of the plurality of nucleic acid scaffolds in the absence of the first candidate molecule and the plurality of second candidate molecules;
(c) contacting the plurality of nucleic acid scaffolds with the first candidate molecule linked to a first glue sequence, thereby anchoring the first candidate molecule to each of the plurality of nucleic acid scaffolds, wherein the first glue sequence is complementary to the first molecule binding sequence;
(d) contacting the plurality of nucleic acid scaffolds with the plurality of second candidate molecules each linked to a second glue sequence, thereby anchoring one of the second candidate molecule among the plurality of candidate molecules to each of the plurality of nucleic acid scaffolds, wherein the second glue sequence is complementary to the second molecule binding sequence, thereby forming a plurality of screening nucleic acid scaffolds, wherein the first candidate molecule and the second candidate molecule of each of the plurality of screening nucleic acid scaffolds are positioned such that the first candidate molecule and the second candidate molecule are in close proximity with each other;
(e) determining elongation lengths for each of the plurality of screening nucleic acid scaffolds in response to the force of 0.01 pN to 50 pN by repeating (d); and
(f) calculating a difference between the reference elongation length and the elongation length of each screening nucleic acid scaffold among the plurality of screening nucleic acid scaffolds,
whereby a differential between the elongation length of a screening nucleic acid scaffold and the reference elongation length indicates that the first candidate molecule and the second candidate molecule anchored to the screening nucleic acid have a binding interaction to each other at the force applied, and whereby the absence of the differential indicates absence of binding interaction between the first candidate molecule and the second candidate molecule at the force applied.
Embodiment 17. The method of Embodiment 16, wherein at least two of the nucleic acid scaffolds comprise barcode sequences that are located at non-identical positions relative to each other.
Embodiment 18. The method of Embodiment 16, wherein at least two of the nucleic acid scaffolds comprise a barcode sequence that are non-identical relative to each other.
Embodiment 19. The method of Embodiment 17 or 18, further comprising determining identity of the hairpin nucleic acid based on the barcode prior to (b).
Embodiment 20. The method of Embodiment 19, wherein the identity of the hairpin nucleic acid is determined by detecting the position of the one or more modified nucleotides in the barcode.
Embodiment 21. The method of Embodiment 20 further comprising resolving a holiday junction, wherein the holiday junction is formed by hybridization of a first junction sequence binding region of the first spacer polynucleotide and a second junction sequence binding region of the second spacer polynucleotide to the first junction sequence and the second sequence, respectively, to form the nucleic acid scaffold.
Embodiment 22. A method of screening binding interactions between a first candidate molecule, a second candidate molecule and a plurality of third candidate molecules, the method comprising:
(a) providing a device that comprises:
   (i) a chamber disposed within the device, wherein the chamber comprises a bottom surface,
   (ii) a force application mechanism for stretching the contiguous polynucleotide sequence along the axis to form the nucleic acid scaffold, and
   (iii) a nucleic acid scaffold positioned along an axis perpendicular to the bottom surface of the chamber of the device, wherein the nucleic acid scaffold comprises the nucleic acid scaffold of any one of Embodiments 1-5, wherein the nucleic acid scaffold is linked to a bead at one end and a feature of the bottom surface of the chamber of the device at the other end;
(b) determining a reference elongation length of the nucleic acid scaffold in the absence of the first candidate molecule and the second candidate molecules by:
   (i) applying a force of 0.01 pN to 50 pN to the bead attached to the nucleic acid scaffold via the force application mechanism along the axis perpendicular to the bottom surface of the chamber of the device, wherein the nucleic acid scaffold is configured to unfold in response to the force applied, thereby resulting in a change in position of the bead attached to the nucleic acid scaffold along the axis, and
   (ii) measuring the change in position of the bead along the axis via a sensor, thereby determining the reference elongation length of the nucleic acid scaffold in the absence of the first candidate molecule and the second candidate molecule;
(c) contacting the nucleic acid scaffold with the first candidate molecule linked to a first glue sequence, thereby anchoring the first candidate molecule to the nucleic acid scaffold;
(d) contacting the nucleic acid scaffold with the second candidate molecule linked to a second glue sequence, thereby anchoring the second candidate molecule to the nucleic acid scaffold, thereby forming a screening nucleic acid scaffold, wherein the first candidate molecule and the second candidate molecule of the screening nucleic acid scaffold are positioned such that the first candidate molecule and the second candidate molecule are in close proximity with each other;
(e) determining a force required to achieve the same elongation length for the screening nucleic acid scaffold as the reference elongation length by repeating (d), wherein the force is in a range of from 0.01 pN to 50 pN;
(f) contacting the plurality of third candidate molecules to the screening nucleic acid scaffold;
(g) determining elongation lengths for the screening nucleic acid scaffolds in the presence of at least one of the plurality of third candidate molecules in response to the same force applied in (e) by repeating (b); and
(h) calculating a difference between the reference elongation length and the elongation length of the screening nucleic acid scaffold, whereby a non-zero differential value indicates presence of a binding interaction between the third candidate molecule, and the first candidate molecule and the second candidate molecule at the force applied, and whereby a differential value of zero indicates absence of the binding interaction between:
   (A) the third candidate molecule and the first candidate molecule,
   (B) the third candidate molecule and the second candidate molecule, or
   (C) the third candidate molecule, and the first candidate molecule and the second candidate molecule, at the force applied.
Embodiment 23: A method of determining a binding interaction between a first candidate molecule and a second candidate molecule, the method comprising:
(a) providing a nucleic acid scaffold of any one of Embodiments Error! Reference source not found.-Error! Reference source not found., wherein the nucleic acid scaffold is positioned along an axis perpendicular to a bottom surface of the chamber of the device;
(b) providing a device that comprises:
   (i) a chamber disposed within the device, wherein the chamber comprises a bottom surface, and
   (ii) a force application mechanism for stretching the contiguous polynucleotide sequence along the axis to form the nucleic acid scaffold;
(c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence;
(d) determining an amplitude of Brownian noise of the nucleic acid scaffold in response to a force of less than 0.01 pN in the absence of the first candidate molecule and the second candidate molecule;
(e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact with each other;
(f) determining an amplitude of Brownian noise of the screening nucleic acid scaffold in response to the same force used in the absence of the first candidate molecule and the second candidate molecule by repeating (d); and
(g) identify events of interaction between the two molecules, wherein the amplitude of Brownian noise is reduced compared to the reference amplitude at the same force in the absence of the molecules.
Embodiment 24: A method of determining a binding interaction between a first candidate molecule, a second candidate molecule and a third candidate molecule, the method comprising:
(a) providing a nucleic acid scaffold of any one of Embodiments Error! Reference source not found.-Error! Reference source not found., wherein the nucleic acid scaffold is positioned along an axis perpendicular to a bottom surface of the chamber of the device;
(b) providing a device that comprises:
   (i) a chamber disposed within the device, wherein the chamber comprises a bottom surface, and
   (ii) a force application mechanism for stretching the contiguous polynucleotide sequence along the axis to form the nucleic acid scaffold;
(c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence;
(d) determining a reference amplitude of Brownian noise of the nucleic acid scaffold in response to a force of less than 0.01 pN in the absence of the first candidate molecule and the second candidate molecule;
(e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact with each other;
(f) contacting a third candidate molecule in solution to the screening nucleic acid scaffold;
(g) determining an amplitude of Brownian noise of the screening nucleic acid scaffold in the presence of the third candidate molecule in response to the same force applied in (d); and
(h) identify events of interaction between the three molecules, wherein the amplitude of Brownian noise is reduced compared to the reference amplitude at the same force in the absence of the three molecules.
Embodiment 25. A kit for determining a binding interaction between a first candidate molecule and a second candidate molecule, the kit comprising:
(a) a nucleic acid that comprises:
   (i) a contiguous polynucleotide sequence comprising:
      (I) a first end that comprises: (A) a first adapter for attaching to a bead, and (B) a first molecule binding sequence;
      (II) a second end that comprises: (A) a second adapter for attaching the nucleic acid to a bottom surface of a device, and (B) a second molecule binding sequence;
      (III) an intermediate portion between the first molecule binding sequence of the first end and the second molecule binding sequence of the second end, wherein the intermediate portion comprises:
         1. a first pin forming sequence comprising a barcode,
         2. a second pin forming sequence that is complementary to the first pin forming sequence, wherein the first pin forming sequence is hybridized to the second pin forming sequence, and
         3. a loop linking the first pin forming sequence and the second pin forming sequence;
   (ii) a first spacer polynucleotide that has a polynucleotide sequence that is complementary to the first pin forming sequence, and
   (iii) a second spacer polynucleotide that has a polynucleotide sequence that is complementary to the second pin forming sequence,
wherein the nucleic acid when attached as a nucleic acid scaffold to the bottom surface of the device requires a force of 10 pN to 30 pN to unfold the hairpin nucleic acid along an axis perpendicular to the bottom surface of the device; and
(b) a bead comprising an anchoring molecule configured to bind to the first adapter of the first end of the contiguous polynucleotide sequence of the nucleic acid.
Embodiment 26. The kit of Embodiment 25, wherein each, the first molecule binding sequence and the second molecule binding sequence, independently comprises a small hairpin nucleic acid having a size in a range of from 5 to 100 bases.
Embodiment 27. The kit of Embodiment 25 or 26 further comprising:
(a) a first glue sequence comprising a first active group that is configured to be linked with a first candidate molecule, wherein the first glue sequence is complementary to the first molecule binding sequence; and
(b) a second glue sequence comprising a second active group that is configured to be linked with a second candidate molecule, wherein the second glue sequence is complementary to the second molecule binding sequence.
Embodiment 28. The kit of any one of Embodiments 25-27 further comprising at least one of the first candidate molecule and the second candidate molecule.
Embodiment 29. The kit of any one of Embodiments 25-28, wherein
(a) the first candidate molecule is linked to the first glue sequence; and
(b) the second candidate molecule is linked to the second glue sequence.
Embodiment 30. The kit of any one of Embodiments 25-29 further comprising a third candidate molecule.
Embodiment 31. The kit of any one of Embodiments 25-30, wherein the first spacer polynucleotide and the second spacer polynucleotide are linked to each other by a spacer loop sequence, wherein the spacer loop sequence is hybridized to the loop.

The invention provides a scaffold comprising:
(a) a first end that comprising a first molecule binding sequence,
(b) an intermediate nucleic acid sequence, said intermediate nucleic acid sequence, comprising:
   (i) a folded sequence comprising at least one barcode, a sequence A at a 5′ end and a sequence B at a 3′end, the sequence A and the sequence B being different and non-complementary,
   (ii) a double stranded nucleic acid sequence comprising a sequence complementary to the folded sequence, juxtaposed at one extremity, with a sequence A′ complementary to the sequence A and a sequence B′ complementary to the sequence B, and
(c) a second end comprising a second molecule binding sequence.

In a preferred embodiment, the double stranded nucleic acid sequence comprising a sequence complementary to the folded sequence comprises a loop.

The invention comprises a scaffold
wherein the folded sequence comprising at least one barcode is bound to the double stranded nucleic acid sequence comprising a sequence complementary to the sequence fold as a hairpin structure via A binding to A′ and B binding to B′ only (Holiday structures).
A form wherein the sequence folded as a hairpin structure comprising at least one barcode is hybridized to the double stranded nucleotide comprising a sequence complementary to the sequence fold as a hairpin structure (invaded, final structures).
The increase in the length of the scaffold (and therefore of the amplitude of the Brownian noise) allows measuring subtle interactions between for example two proteins.

Another main object provided herein is a first candidate molecule linked to a first glue sequence, wherein the first glue sequence hybridizes to the first molecule binding sequence; and a second candidate molecule linked to a second glue sequence, wherein the second glue sequence hybridizes to the second molecule binding sequence, alone or bound to the scaffold according to the present invention.

The first and second candidate molecule linked to a first and second glue sequences, respectively can be any candidate molecule, preferably candidate molecules binding to each other and/or binding to another molecule. Thus, the scaffold of the invention can be used to identify binding molecules, to quantify a binding etc.

In some embodiments, the scaffold of the invention wherein the double stranded nucleic acid complementary to the folded sequence has a number of bases between 10 and 3000 bases, advantageously 100 bases, is provided.

The scaffold of the invention is provided in combination with a molecule specifically binding to the barcode, advantageously an antibody specific for a modified nucleotide, in particular an antibody specific for an epigenetically modified nucleotide.

In particular embodiments, the scaffold of the invention is provided wherein the barcode comprises at least one modified base, advantageously a methylated cytosine, advantageously at least one SEQ ID NO: 3 wherein the second cytosine is epigenetically modified in E. coli or wherein the second cytosine is methylated in E. coli.

Alternatively, a base modification can be obtained in vitro and the modified base inserted into a hairpin in vitro.

Nucleotide sequences of the scaffold of the invention are assembled first so that A hybridizes with A′, B hybridizes with B′; the strands of the double stranded nucleic acid sequence can then hybridize with the folded sequence upon unfolding (opening) of said folded sequence folded. This requires to pull the folded structure with a force ranging from 1 to 25 pN.

The invention provides as another main object:
a device or a platform comprising:
the scaffold according to the invention, wherein the first end is attached to a bead, advantageously a magnetic bead, the second end is attached to a bottom surface of the device, in an electrically conductive solution.
   a force application mechanism, advantageously a magnet,
   a detection system for detecting the position of the bead in real time.

The invention provides:
a device or a platform comprising
   a chamber with a bottom surface,
   a force application mechanism, advantageously a magnet,
   a detection system for detecting the position of a bead in real time, and separately, a kit comprising a bead, advantageously a magnetic bead, the scaffold of the invention comprising a barcode, said barcode consisting of a number and a position of a modified basis, an antibody specific for the modified base, an electrically conductive solution, a set of glue sequences that can be glued to a candidate molecule or molecules of interest.

Accordingly, the invention provides a method for preparing the scaffold of the invention by first detecting the number and position of a modified base (barcode) in a folded sequence said folded sequence being attached to a bead and to a bottom surface of a device. This is achieved by measuring a bead position in real time while
1) exerting a force moving the bead to unfold the folded sequence,
2) stopping the force and letting the folded sequence refold,
3) contacting the folded sequence refolded or said folded sequence unfolded with an antibody specific for the modified base,
4) exerting a force to move the bead, decoding number and position of the modified base from the bead positions.

Accordingly, the invention provides a method for preparing a scaffold of the invention further comprising:
a step of contacting a folded sequence comprising a sequence A at a 5′ end and a sequence B at a 3′end, the sequence A and the sequence B being different and non-complementary, and a decoded barcode with a double stranded nucleic acid sequence comprising a sequence complementary to the folded sequence, juxtaposed at one extremity, with a sequence A′ complementary to the sequence A and a sequence B′ complementary to the sequence B,
resulting in the binding of A, with A′ and B with B,
a step of exerting a force of 5 to 25 pN to open the folded sequence comprising a decoded barcode, triggering hybridization of the strands of the double stranded nucleotide to the folded sequence.

The hybridization of the strands of the double stranded nucleic acid to the folded sequence (strand invasion) is measured by detecting a jump in the bead position due to an increase in the length of the scaffold.

The invention provides as another object, a kit comprising any one of the scaffolds of the invention said scaffold comprising a barcode and said barcode comprising at least one modified basis, an antibody specific for the modified basis of the barcode in the scaffold, an electrically conductive solution.

The kit can further comprise a double stranded nucleotide comprising a sequence complementary to the folded sequence or a set thereof of different sizes.

The kit comprising up to 20, 50, 100, 200, 500, 1000, 2000, 5000, 10000, 20000, 50000, 100000, 200000, 500000, 1000000 scaffolds of the invention, advantageously 10000 scaffolds.

The invention provides as another object, a kit comprising a bead, any one of the scaffolds of the invention said scaffold comprising a barcode and said barcode comprising at least one modified basis, an antibody specific for the modified basis of the barcode in the scaffold, an electrically conductive solution.

The invention provides as another object, a kit comprising a bead, any one of the scaffolds of the invention, said scaffold comprising a barcode and said barcode comprising at least one modified basis, an antibody specific for the modified basis of the barcode in the scaffold, a set of double stranded nucleotide comprising a sequence complementary to the folded sequence, an electrically conductive solution.

The device, the scaffold, the kit of the invention for use in a binding assay.

The device, the scaffold or the kit of the invention for use in a binding assay, advantageously for use in a binding assay in real time.

### EXAMPLES

For a better understanding of the present disclosure and of its many advantages, the following examples are given by way of illustration and without limiting the scope of this disclosure.

### Example 1. Identifying Hairpin Nucleic Acids

The sequence of the first hairpin nucleic acid precursor, containing multiple CCwGG (SEQ ID NO: 3) sequences spaced within the fragment or randomly generated sequence (all the sequences are different), was synthesized and cloned into a plasmid for propagation within bacteria (E. coli). The sequence CCwGG (SEQ ID NO: 3) was methylated in vivo by the E. coli DCM methylase. The plasmid was isolated from bacteria using commercial plasmid extraction kit and 5 μg of the plasmid was digested with BsaI. Digested fragments of about 300 base pairs containing the spaced methylated cytosines were recovered by agarose gel electrophoresis. Then, first Y-shape forming polynucleotide sequence comprising a first end and a first junction sequence (from 5′ to 3′) of the hairpin nucleic acid that comprises biotin at the 5′-end (SEQ ID NO: 11), a second Y-shape forming polynucleotide sequence encoding a second junction sequence, a second molecule binding sequence and a second adapter 107 (SEQ ID NO: 8) of the hairpin nucleic acid, and a loop (SEQ ID NO: 7) were ligated on the fragment to form the hairpin nucleic acid. The final hairpin nucleic acids were gel purified. The hairpin nucleic acid was then attached on MyOne bead (paramagnetic beads from Dynabead of 1 μm in size) by the biotin present at the first end to prepare anchored hairpin nucleic acid. The anchored hairpin nucleic acid was immobilized to a feature of a bottom surface of a flow cell, wherein the feature comprised an oligonucleotide sequence (SEQ ID NO: 9) that was complementary to the second adapter.

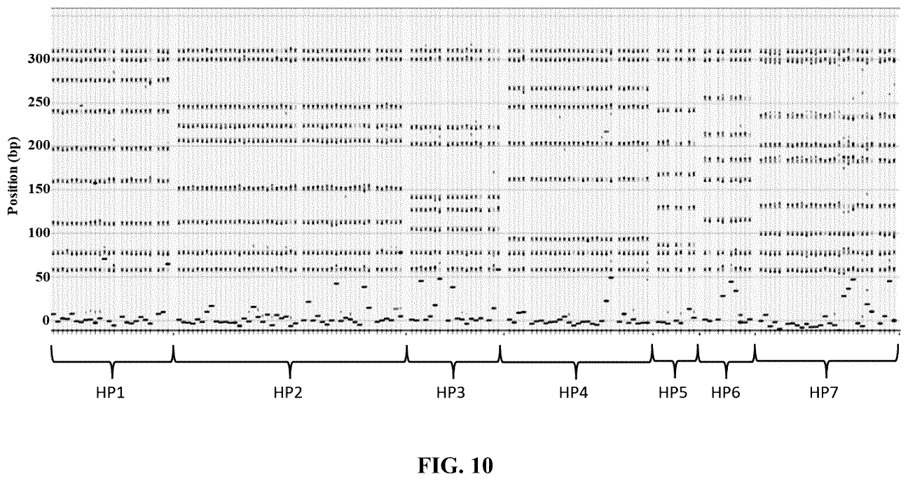

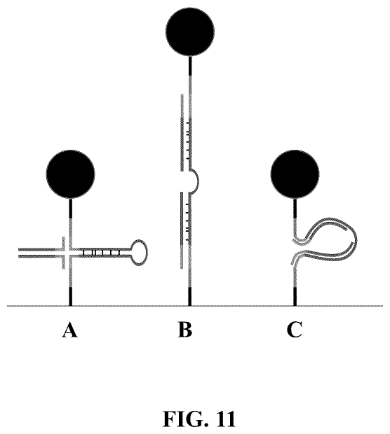

To demonstrate the capacity to detect multiple interaction at the same time, seven plasmids were mixed, each containing a different barcode (the position of the methylated cytosines is different, see FIG. 4 for example of possible barcodes) (SEQ ID NOS: 25-32). The plasmids were mixed in equimolar concentration, and all processed together within the same test tube to make hairpin nucleic acids as described previously. The hairpin nucleic acids were then positioned between beads and features of the flow cell. The flow cells were then injected with an antibody fragment (Fab) against 5-mC (Fab fragment derived from the clone ICC/IF from Diagenode). Blockage patterns were observed for each bead based on binding of the antibody to the methylated cytosine of the hairpin nucleic acid when a force was applied. At least 100 test cycles were performed to detect the positions of the methylated cytosines for all the hairpin nucleic acids. Each blockage pattern was unique for the specific barcode. FIG. 9 shows exemplary trace showing blocking pattern for one of the hairpin nucleic acid and FIG. 10 shows the classification of these seven sequences according to their barcode. The method described herein provides that hairpin nucleic acids can be properly assigned to one of the sequences and they can be demultiplexed by the method described. Accordingly, the method can be used to decode barcodes of the hairpins that is required to probe multiple interactions at the same time.

### Example 2. Preparing Nucleic Acid Scaffold

To demonstrate capacity to produce the scaffold with the spacer polynucleotide sequence (SEQ ID NO: 33), the hairpin nucleic acid corresponding to SEQ ID NO: 32 were prepared as described in Example 1 and immobilized between beads and features of a bottom surface of a flow cell. In parallel, spacer polynucleotides were prepared by ligating a BsaI digested first hairpin nucleic acid precursor with two specific Y-shape forming polynucleotide sequences at one end and a spacer loop at another end, wherein the two polynucleotide sequences include: (a) a first Y-shape forming polynucleotide sequence encoding a first junction sequence (from 5′ to 3′), and (b) a second Y-shape forming polynucleotide sequence encoding a second junction sequence (from 5′ to 3′). Once purified on agarose gel, the specific spacer polynucleotide was injected into the flow cell to form a 4-way junction based on a polynucleotide sequence of a hairpin nucleic acid that was identified by decoding barcode using the method provided in Example 1.

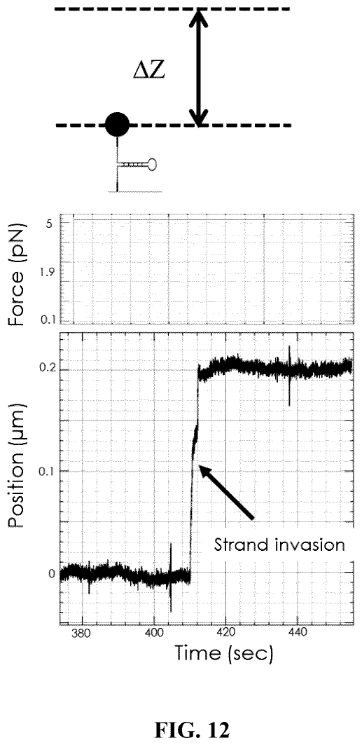

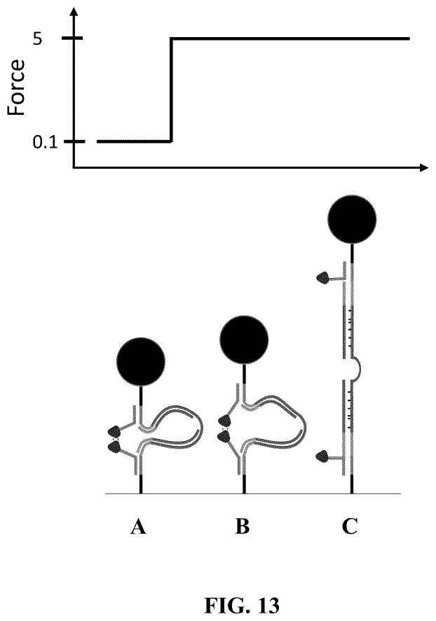

This allows to anchor the spacer polynucleotides to form a 4-ways junction (also referred as Holiday junction) with the hairpin nucleic acids. Then, a force of 5 pN was applied to the bead to resolve holiday junctions and cause strand invasion. The position of the beads was recorded during the injection. Upon the resolution of the Holyday junction, the Z position of bead will move according to the size of the hairpin upon successful invasion. The successful invasion corresponds to a movement of the position of the bead by about 200 nm in this example, which corresponds to the length of the dsDNA linker (600 bp at 0.3 nm/bp=180 nm). FIG. 12 shows exemplary recording of the position of the bead before and after the strand invasion.

### Example 3. System for Determining Binding Kinetics Between Two Substrates Forming Non-Homologous End Joining (NHEJ) Complex

The system described in Example 1 and Example 2 was used for determining binding kinetics between two substrates and proteins that form a Non-Homologous End Joining (NHEJ) complex. The NHEJ complex recognizes double stranded blunt ends and repair these lesions in the cell. Accordingly, two substrates, each comprising a single stranded carrier polynucleotide and a candidate molecule comprising a double stranded blunt end were prepared, wherein the single stranded carrier polynucleotide was complementary to a molecule binding sequence of a nucleic acid scaffold. The double stranded blunt end was prepared by annealing twice two oligos together (SEQ ID NOS: 14 and 15, as well as SEQ ID NOS: 17 and 18 at 10 μM each in PBS).

Because each of the proposed sequences contained the glue sequence for first and second molecule binding sequence respectively, the substrates could be directed to a specific location on the nucleic acid scaffold. Accordingly, the two substrates for the NHEJ complex were anchored to two molecule binding sequences of the nucleic acid scaffold to form a screening nucleic acid scaffold. The excess substrates were washed away. A reference elongation length of a screening nucleic acid scaffold was determined by applying 2 pN force in the absence of the substrates.

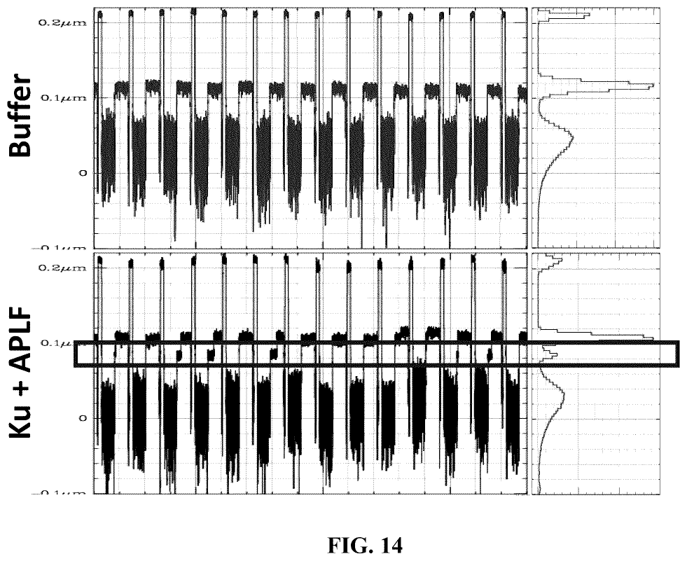

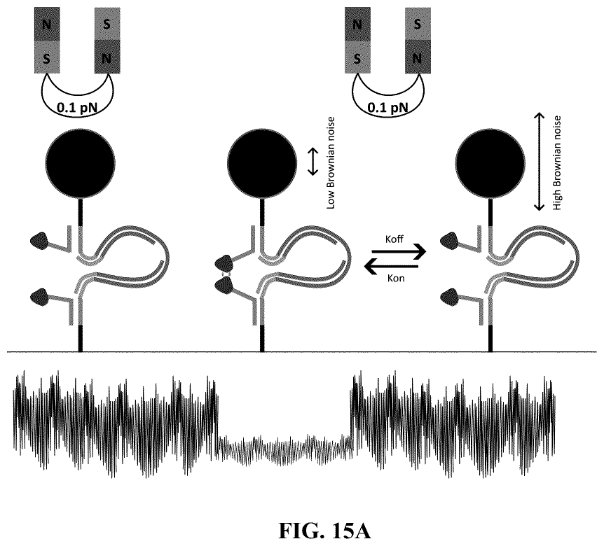

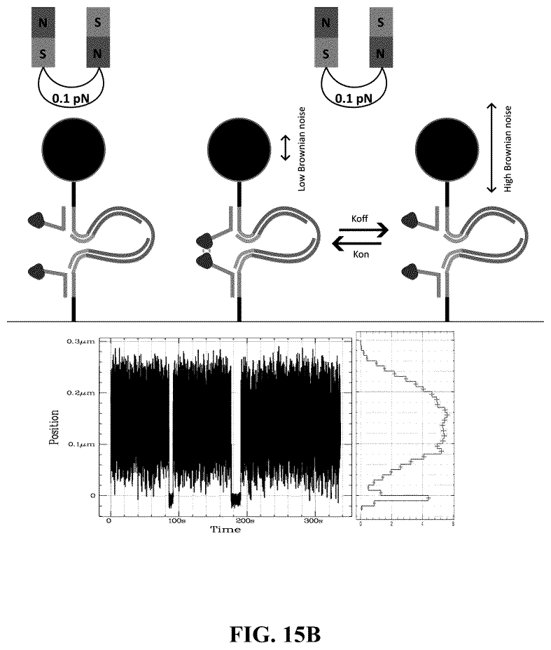

Next, two candidate proteins, KU70/80 complex as well as the APFL protein together into PBS 1X, were injected within the flow cell. The flow was stop and force cycles experiments were initiated. Blockage traces were recorded at 0.1 pN and 2 pN. At 0.1 pN force, the screening nucleic acid scaffold was unstructured, which allowed the two blunt ends to be brought in close proximity. If there was no protein present in the flow cell that could hold the two blunt ends together, increasing the force to 2 pN would have resulted in full stretching of the screening nucleic acid scaffold. Similarly, if only the KU70/80 or APFL protein were to be loaded independently, the scaffold would have stretched fully at 2 pN indicating that, alone by themselves, they were not able to facilitate a binding interactions between the two blunt ends. However, one or more transient blockages were observed for screening nucleic acid scaffold at 2 pN when both proteins were injected together, until the interaction was broken, and the screening nucleic acid scaffold was fully stretched (FIG. 14). A presence of the one or more transient blockages indicates that the KU70/80 and APFL, together, forms a complex on the two substrates that are double stranded. Because the method was a non-destructive process, the force cycles were repeated multiple times to further probe the interaction.

The experiment shows that the system can be used for determining Kₒₙ (number of cycles where the interaction was observed) by testing various concentration of the proteins as well as K_{off} at a specific force (how long it takes before breaking the complex). The Kₒₙ and K_{off} can be used for calculating Kd.

### Example 4. System for Determining Binding Interactions Between Two Substrates Forming Non-Homologous End Joining (NHEJ) Complex

The system described in Example 1 and Example 2 are used for determining binding interactions between blunt end substrate located on the scaffold and proteins that form a Non-Homologous End Joining (NHEJ) complex. The experiment is roughly performed similar to the method described in Example 3 except a constant force is applied to probe the binding interaction between the two blunt ends rather than force cycles. Briefly, a reference amplitude of the Brownian movement displacement in the Z axis of a nucleic acid scaffold is determined by applying a constant force. This corresponds roughly to the size of the DNA scaffold (about 180 nm for a scaffold of 600 bp in this example). Next, the flow cell comprising the nucleic acid scaffold is injected with a combination of KU70/80 and APFL. An interaction between the blunt DNA substrate and the protein complex generates a screening nucleic acid scaffold (FIG. 15B). The amplitude of the Brownian movement of a screening nucleic acid scaffold is determined throughout the experiment under constant force. Over time and upon formation of the NHEJ complex on the blunt end substrates, the amplitude of the Brownian movement is drastically reduced to only few nanometres due to shortening of the scaffold. The amplitude of the Brownian movement will remain low until the interaction is broken. The experiment shows that the system can detect interaction even at low force (<5 pN) while maintaining the constant force.

Next, Kₒₙ is determined by counting the number of events observed versus the concentration of the NHEJ complex. Also, it is possible to measure the K_{off} of this interaction by taking into consideration the average amount of time for which the NHEJ complex interacts for all the event detected.

### Example 5. System for Determining Binding Interactions Between Restriction Enzymes and T4 DNA Ligase

The system described in Example 1 and Example 2 are used for determining activity of a restriction enzyme as well as T4 DNA ligase. Two candidate molecules are used, which includes: (a) a first candidate molecule comprising a single stranded polynucleotide complementary to the first molecule binding sequence as well as a portion double stranded which contains the restriction site recognition sequence (BsaI, GGTCTC (SEQ ID NOS: 19 and 20)) and the 5′ overhang TATC; and (b) a second candidate molecule comprising a single stranded polynucleotide complementary to the second molecule binding sequence as well as a double stranded part terminated at the 5′ by a four bases overhang sequence (GATA (SEQ ID NOS: 21 and 22)). A reference elongation length of a nucleic acid scaffold is determined by applying a constant force of 0.1 pN in the absence of candidate molecules. Next, a screening nucleic acid scaffold is generated by anchoring the two candidate molecules to the nucleic acid scaffold. Then, the flow cell comprising the screening nucleic acid scaffold is injected with T4 DNA ligase to generate a screening nucleic acid scaffold. Elongation length of the screening nucleic acid scaffold is determined by applying a constant force of 0.1 pN and the amplitude of the Brownian motion is determined. No change in the amplitude of the Brownian motion indicates that the ligation didn't occur between the two candidate molecules. In contrast, when ligation occurred, the amplitude of the Brownian motion will reduce due to the shortening of the scaffold and indicates a successful ligation between the two candidate molecules. One indication of a successful ligation is that the scaffold can't be stretched even at more than 5 pN to the reference elongation length.

The experiment also provides a roadmap for determining kinetics of the reaction by changing the concentration of T4 DNA ligase and determining the time it takes to ligate the two substrate molecules.

The experiment also provides a roadmap for determining the kinetic of a restriction enzyme (BsaI). For example, the restriction enzyme can be injected to the flow cell containing ligated candidate molecules. A digestion of the ligated substrate would result in a sudden jump in the Z position of the bead of about 200 nm and restoration of the elongation length to that of reference elongation length.

### Example 6. System for Determining Binding Interactions Between Two Proteins

The system described in Example 1 and Example 2 are used for determining binding interactions between two candidate molecules (proteins). Briefly, a reference elongation length of a nucleic acid scaffold present in a flow cell is determined at a constant force. Next, a single stranded polynucleotide containing the complementary sequence of the first molecule binding sequence as well as an amine group at the 5′ end (SEQ ID NO: 23) is coupled to candidate protein (RBD protein from the SARV-CoV2 virus) by a covalent conjugation (esterification via EDC). In parallel, a second single stranded polynucleotide containing the complementary sequence of the second molecule binding sequence as well as an amine group at the 3′ end (SEQ ID NO: 34) is coupled to candidate protein (Ace2 receptor) by a covalent conjugation (esterification via EDC). A first candidate molecule conjugated to a first polynucleotide and a second candidate molecule covalently conjugated to a second polynucleotide are sequentially injected into the flow cell to anchor them on to the nucleic acid scaffold and form a screening nucleic acid scaffold. Then, a force of 2 pN is applied to determine the maximal elongation length. The force is then reduced to 0.1 pN or 0.01 pN to allow the two proteins to interact. If the two candidate molecules have binding interaction between them, upon increasing the force back to 2 pN, the elongation observed is going to be less than the full extension of the screening nucleic acid scaffold. If the two candidate molecules have no binding interaction between them or weaker binding interaction between them than the applied force, the nucleic acid scaffold will fully stretch. The experiment provides a roadmap for: (a) performing the experiment at different forces; (b) performing the experiment with different buffer conditions; and (c) determining strength of the interaction between the two proteins.

### Example 7. System for Determining Binding Interactions Between a Protein and an Aptamer

The system described in Example 1 and Example 2 are used for determining binding interactions between two candidate molecules, a protein and an aptamer. Briefly, a reference elongation length of a nucleic acid scaffold present in a flow cell is determined at a constant force. Two candidate molecules are engineered, which include: (a) a single stranded polynucleotide containing the complementary sequence of the first molecule binding sequence as well as an amine group at the 5′ end (SEQ ID NO: 23) coupled to a candidate protein (RBD protein from the SARV-CoV2 virus) by a covalent conjugation (esterification via EDC), (b) a second candidate molecule (aptamer) comprising the sequence complementary to the second molecule binding sequence as well as a polynucleotide sequence encoding an aptamer against the RBD protein (SEQ ID NO: 10). Then, the first candidate molecule and the second candidate molecule are sequentially injected into the flow cell to anchor them on to the nucleic acid scaffold and form a screening nucleic acid scaffold. A constant force of 2 pN is applied to determine the maximal elongation length of the screening nucleic acid scaffold. The force is then reduced to allow the two candidate molecules to interact. If the two candidate molecules have binding interaction between them, the displacement of the bead, when the force is going to be increased to 2 pN of force, is going to be less than the maximal length. If the two candidate molecules have no binding interaction between them or weaker binding interaction between them than the applied constant force, the maximal extension is observed. The force cycle can be repeated to determine binding kinetics between the two candidates.

### Example 8. System for Determining Binding Interactions Between a Bispecific Antibody and Candidate Antigens

The system described in Example 1 and Example 2 are used for determining binding interactions between a bispecific antibody and two antigens. Briefly, a reference trace is recorded for a nucleic acid scaffold by applying a constant force of 0.1 pN for two minutes to determine the amplitude of the Brownian motion. Next, the flow cell comprising the nucleic acid scaffold was sequentially injected with two candidate molecules to anchor them to the nucleic acid scaffold and form a screening nucleic acid scaffold. The two candidate molecules include: (a) a first candidate molecule (polynucleotide) comprising the sequence complementary to the first molecule binding sequence (first glue sequence) as well as a single stranded region with one cytosine modified with a methyl group (5-methyl cytosine) (SEQ. ID NO: 19); and (b) a second candidate molecule (polynucleotide) comprising the sequence complementary to the second molecule binding sequence (second glue sequence) as well as a single stranded region with one cytosine modified with a methyl group (5-methyl cytosine) (SEQ. ID NO: 21). Then, the bispecific antibody against the 5-mC modification (ICC clone from Diagenode, catalogue number C15200003) is injected into the flow cell. Binding events are determined by recording a trace for the screening nucleic acid scaffold at the same constant force of 0.1 pN for 30 minutes. A decrease in the noise emerging from a bead indicates binding events.

### Example 9. System for Determining Structural Characteristics of an RNA

The system described in Example 1 and Example 2 are used for determining binding interactions between two candidate molecules. Briefly, a candidate RNA molecule having a secondary structure (e.g., preQ1 structure) is engineered, which includes a sequence complementary at the 5′ end to the first molecule binding sequence as well as a sequence complementary to the second molecule binding sequence at the 3′ end. Then, the candidate molecule is injected into the flow cell to anchor it on to the nucleic acid scaffold, therefore making a bridge between the first molecule binding sequence and second molecule binding sequence. When a force is applied to the system, it results in the unfolding of the RNA molecule. Next, a third candidate molecule that can bind to the RNA secondary structure (e.g., PreQ1 ligand) is injected at different concentrations into the flow cell. Next, ramp cycles, force cycle or application of step constant force are performed to determine the binding kinetic of the third candidate molecule to the RNA structure and determine if the binding of the third candidate molecule stabilize or destabilize the structure. The experiment provides roadmap to determine multiplex different RNA secondary structures within the same flow cell, without changing the cartridge.

The experiment provides a roadmap to determine effect of one or more candidate molecules on the secondary structure of the RNA, for example, by injecting the one or more candidate molecules into the flow cell and repeating the ramp cycles, force cycles or the application of step constant force as described above.

### Example 10. Identifying Hairpin Nucleic Acids

The sequence of the first hairpin nucleic acid precursor, containing multiple CCwGG (SEQ ID NO: 3) sequences spaced within the fragment, was synthesized and cloned into a plasmid for propagation within bacteria (E. coli). The sequence CCwGG (SEQ ID NO: 3) was methylated in vivo by the E. coli DCM methylase. The plasmid was isolated from bacteria using commercial plasmid extraction kit and 5 μg of the plasmid was digested with BsaI. Digested fragments of about 300 base pairs containing the spaced methylated cytosines were recovered by agarose gel electrophoresis. Then, first Y-shape forming polynucleotide sequence comprising a first end and a first junction sequence (from 5′ to 3′) of the hairpin nucleic acid that comprises biotin at the 5′-end (SEQ ID NO: 11), a second Y-shape forming polynucleotide sequence encoding a second junction sequence, a second molecule binding sequence and a second adapter 107 (SEQ ID NO: 8) of the hairpin nucleic acid, and a loop (SEQ ID NO: 7) were ligated on the fragment to form the hairpin nucleic acid. The final hairpin nucleic acids were gel purified. The hairpin nucleic acid was then attached on MyOne bead (paramagnetic beads from Dynabead of 1 μm in size) by the biotin present at the first end to prepare anchored hairpin nucleic acid. The anchored hairpin nucleic acid was immobilized to a feature of a bottom surface of a flow cell, wherein the feature comprised an oligonucleotide sequence (SEQ ID NO: 9) that was complementary to the second adapter.

To demonstrate the capacity to detect multiple interaction at the same time, seven plasmids were mixed, each containing a different barcode (the position of the methylated cytosines is different, see FIG. 4 for example of possible barcodes). The plasmids were mixed in equimolar concentration, and all processed together within the same test tube to make hairpin nucleic acids as described previously. The hairpin nucleic acids were then positioned between beads and features of the flow cell. The flow cells were then injected with an antibody fragment (Fab) against 5-mC (Fab fragment derived from the clone ICC/IF from Diagenode). Blockage patterns were observed for each bead based on binding of the antibody to the methylated cytosine of the hairpin nucleic acid when a force was applied. At least 100 test cycles were performed to detect the positions of the methylated cytosines for all the hairpin nucleic acids. Each blockage pattern was unique for the specific barcode. FIGS. 13A and 13B show exemplary trace showing blocking pattern for one of the hairpin nucleic acids. The method described herein provides that hairpin nucleic acids can be properly assigned to one of the sequences and they can be demultiplexed by the method described. Accordingly, the method can be used to decode barcodes of the hairpins that is required to probe multiple interactions at the same time.

### Example 11. Preparing Nucleic Acid Scaffold

Six hairpin nucleic acids attached to beads and features of a bottom surface of a flow cell were prepared as described in Example 1. In parallel, spacer polynucleotides were prepared by ligating a BsaI digested first hairpin nucleic acid precursor with two specific Y-shape forming polynucleotide sequences at one end and a spacer loop at another end, wherein the two polynucleotide sequences include: (a) a first Y-shape forming polynucleotide sequence encoding a first junction sequence (from 5′ to 3′), and (b) a second Y-shape forming polynucleotide sequence encoding a second junction sequence (from 5′ to 3′). Once purified on agarose gel, the specific spacer polynucleotide was injected into the flow cell to form a 4 -way junction based on a polynucleotide sequence of a hairpin nucleic acid that was identified by decoding barcode using the method provided in Example 1.

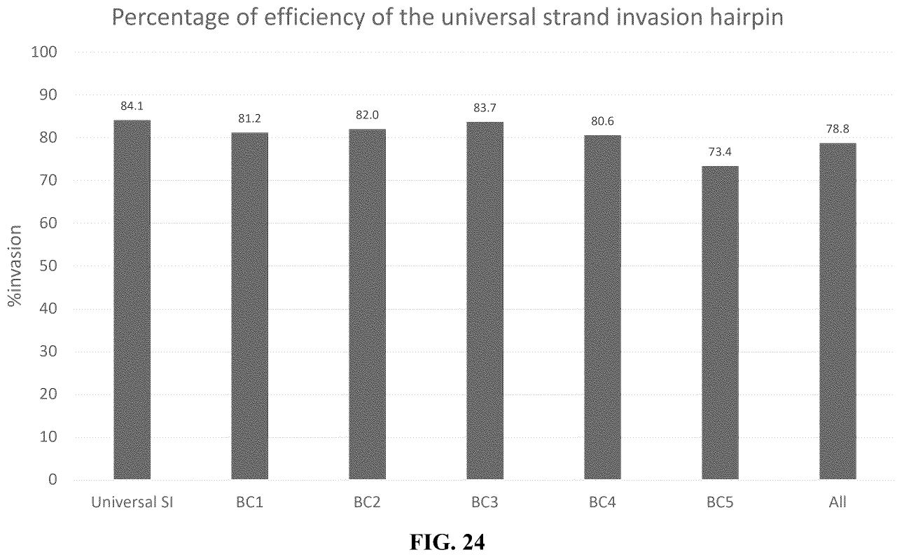

This allows to anchor the spacer polynucleotides to form a 4-ways junction (also referred as Holiday junction) with the hairpin nucleic acids. Then, a force of 5 pN was applied to the bead to resolve holiday junctions and cause strand invasion. The position of the beads was recorded during the injection. Upon the resolution of the Holyday junction, the Z position of bead would move according to the size of the hairpin upon successful invasion. The successful invasion corresponds to a movement of the position of the bead by about 200 nm in this example, which corresponds to the length of the dsDNA linker (600 bp at 0.3 nm/bp=180 nm). FIG. 23 shows exemplary recording of the position of the bead before and after the strand invasion. FIG. 24 shows efficiency of strand invasion in six hairpin nucleic acids comprising universal barcode sequences. An analysis of FIG. 24 indicates that the presence of single nucleotide polymorphism differences between the universal strand invasion and the barcode sequence (SEQ ID NO: 38) (due to the change in the sequence CawGG (SEQ ID NO: 54) into CCwGG (SEQ ID NO: 3)), doesn't affect the efficiency of the strand invasion. This allows us to use a single strand invasion hairpin for all the barcodes.

### Example 12. System for Determining Binding Kinetics Between Two Substrates Forming Non-Homologous End Joining (NHEJ) Complex

The system described in Example 1 and Example 2 was used for determining binding kinetics between two substrates and proteins that form a Non-Homologous End Joining (NHEJ) complex. The NHEJ complex recognizes double stranded blunt ends and repairs these lesions in the cell. Accordingly, two substrates, each comprising a single stranded carrier polynucleotide and a candidate molecule comprising a double stranded blunt end were prepared, wherein the single stranded carrier polynucleotide was complementary to a molecule binding sequence of a nucleic acid scaffold. The two double stranded blunt end adapters were prepared by annealing in two separate tubes two oligos together (SEQ ID NOS: 14 and 15, as well as SEQ ID NOS: 17 and 18 at 10 μM each in a buffer containing: 50 mM NaCl, 10 mM Tris-HCl, 10 mM MgCl2, 1 mM DTT at pH 7.9 @ 25° C.).

Because the proposed system was based on the protein complex recognizing blunt ends DNA, the substrates could be directed to a specific location on the nucleic acid scaffold. Accordingly, the two substrates for the NHEJ complex were anchored to two molecule binding sequences of the nucleic acid scaffold to form a screening nucleic acid scaffold. The excess substrates were washed away. A reference elongation length of a screening nucleic acid scaffold was determined by applying force cycles at three step forces: 0.01, 2 and 20 pN force in the absence of the substrates.

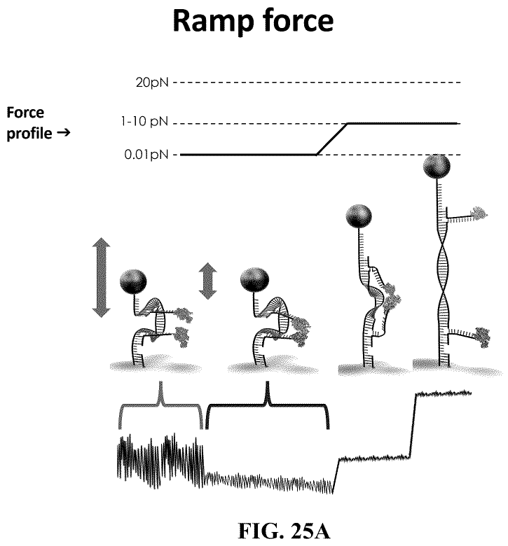

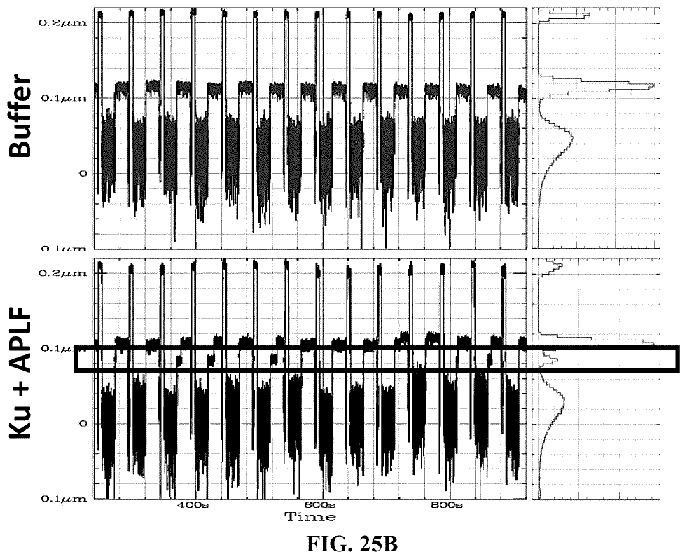

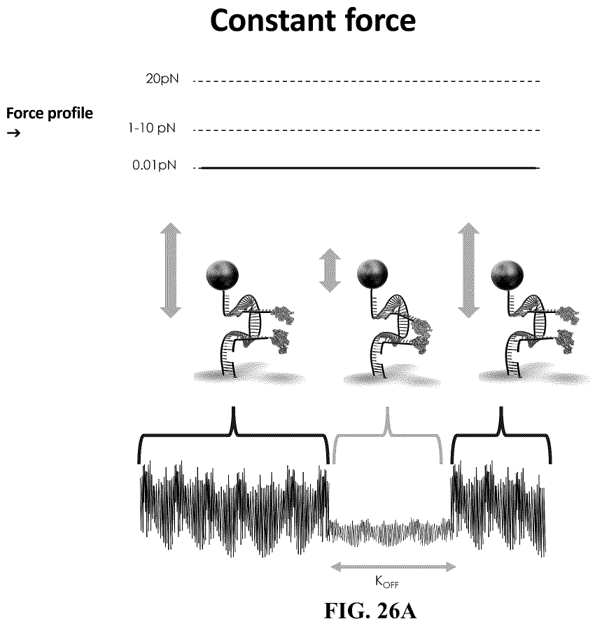

Next, two candidate proteins, KU70/80 complex as well as the APFL protein together into a buffer containing: 20 mM Hepes-KOH PH7.8, 100 mM KCL, 5 mM MgCl2, 1 mM DTT, 0.5 mg/mL BSA, and they were injected within the flow cell. The flow was stop and force cycles experiments were initiated. Force cycles were recorded were the force varies between three plateau: 0.01 pN, 2 pN and 20 pN (FIG. 25A). At 0.01 pN force, the screening nucleic acid scaffold was unstructured, which allowed the two blunt ends to be brought in close proximity. If there was no protein present in the flow cell that could hold the two blunt ends together, increasing the force to 2 pN would have resulted in full stretching of the screening nucleic acid scaffold. Similarly, if only the KU70/80 or APFL protein were to be loaded independently, the scaffold would have stretched fully at 2 pN indicating that, alone by themselves, they were not able to facilitate a binding interactions between the two blunt ends. However, one or more transient blockages were observed for screening nucleic acid scaffold at 2 pN when both proteins were injected together, until the interaction was broken, and the screening nucleic acid scaffold was fully stretched. A presence of the one or more transient blockages indicates that the KU70/80 and APFL, together, forms a complex on the two substrates that are double stranded (FIG. 25B). Because the method was a non-destructive process, the force cycles were repeated multiple times to further probe the interaction.

The experiment shows that the system can be used for determining Kₒₙ (number of cycles where the interaction was observed) by testing various concentration of the proteins as well as K_{off} at a specific force (how long it takes before breaking the complex). The Kₒₙ and K_{off} can be used for calculating Kd.

### Example 13. System for Determining Binding Interactions Between Two Substrates ACE2 and SARS-COV2 RBD

The system described in Example 1 and Example 2 were used for determining binding interactions between blunt end substrate located on the scaffold and proteins that form a Non-Homologous End Joining (NHEJ) complex. FIG. 26A shows a graphical representation of how binding kinetics were determined between the two candidate molecules at constant low force. The experiment was roughly performed similar to the method described in Example 3 except a constant force was applied to probe the binding interaction between the two proteins rather than force cycles. Briefly, a reference amplitude of the Brownian movement displacement in the Z axis of a nucleic acid scaffold was determined by applying a constant force. This corresponds roughly to the size of the DNA scaffold (about 180 nm for a scaffold of 600 bp or about 270 nm for a scaffold of 900 bp in this example). Next, the flow cell comprising the nucleic acid scaffold was injected with ACE2 tagged with an glue nucleotide and RBD with a second glue oligonucleotide. An interaction between the two proteins generated a screening nucleic acid scaffold. The amplitude of the Brownian movement of a screening nucleic acid scaffold was determined throughout the experiment under low constant force. Over time and upon formation of the ACE2-RBD complex, the amplitude of the Brownian movement was drastically reduced to only few nanometres due to shortening of the scaffold (from 900 bp to 300 bp). The amplitude of the Brownian movement remained low until the interaction was broken. The experiment shows that the system can detect interaction even at low force (<5 pN) while maintaining the constant force.

Next, Kₒₙ was determined by counting the number of events observed versus the concentration of the NHEJ complex. Also, it is possible to measure the K_{off} of this interaction by taking into consideration the average amount of time for which the NHEJ complex interacts for all the event detected.

### Example 14. System for Determining Binding Interactions Between a Bispecific Antibody and Candidate Antigens

The system described in Example 1 and Example 2 were used for determining binding interactions between a bispecific antibody and two antigens. Briefly, a reference trace was recorded for a nucleic acid scaffold by applying a constant force of 0.01 pN for two minutes to determine the amplitude of the Brownian motion (FIG. 26B). Next, the flow cell comprising the nucleic acid scaffold was sequentially injected with two candidate molecules to anchor them to the nucleic acid scaffold and form a screening nucleic acid scaffold. The two candidate molecules included: (a) a first candidate molecule (antigen) covalently attached to an oligonucleotide containing the sequence complementary to the first molecule binding sequence (SEQ. ID NO: 19); and (b) a second candidate molecule (antigen) covalently attached to an oligonucleotide containing the sequence complementary to the second molecule binding sequence (SEQ. ID NO: 21). Then, the bispecific antibody was injected into the flow cell. Binding events were determined by recording a trace for the screening nucleic acid scaffold at the same constant force of 0.01 pN for 5 minutes. The force is then increased to 20 pN to remove all interactions and the force is reduced back to 0.01 pN. The exemplary trace is shown in FIG. 26C. As this is a non-destructive assay, this cycle can be repeated as many times required to acquired high quality data. A decrease in the noise emerging from a bead indicates binding events.

### Example 15. System for Determining Binding Interactions Between Two Proteins Via the Presence of a PROTAC or Molecular Glue

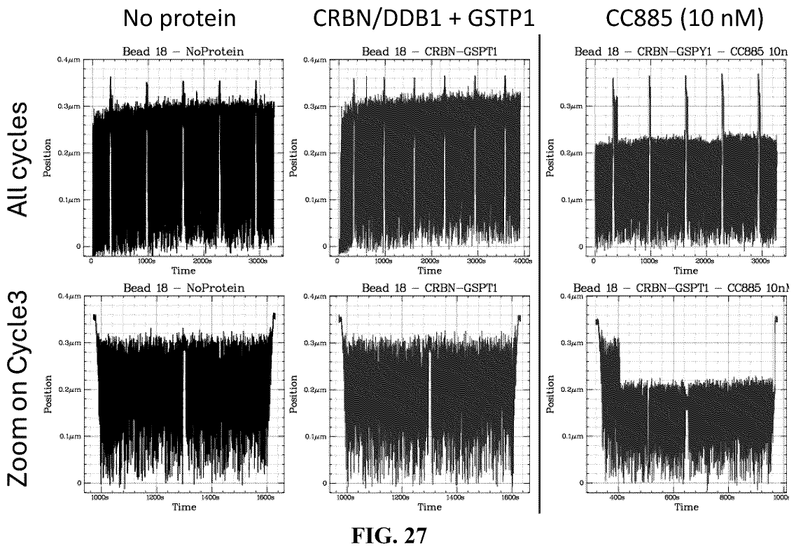

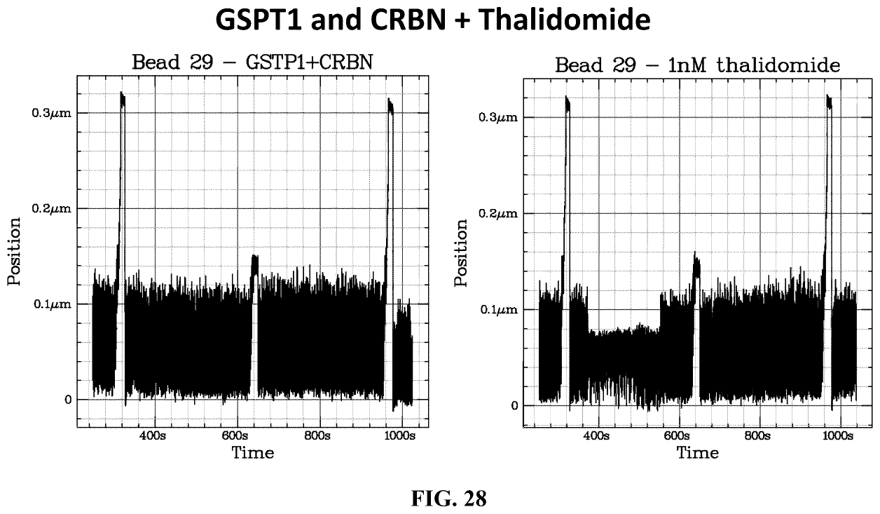

The system described in Example 1 and Example 2 were used for determining binding interactions between two candidate molecules (proteins) ion the presence of a molecular glue. Briefly, a reference elongation length of a nucleic acid scaffold present in a flow cell was determined at a constant force. Next, a single stranded polynucleotide containing the complementary sequence of the first molecule binding sequence as well as an amine group at the 5′ end (SEQ ID NO: 23) was coupled to candidate protein (CRBN and its binding partner DDB1 that form the E3 ligase complex) by a covalent conjugation (esterification via EDC). In parallel, a second single stranded polynucleotide containing the complementary sequence of the second molecule binding sequence as well as an amine group at the 3′ end (SEQ ID NO: 34) was coupled to candidate protein (GSTP1 neo-substrat of CRBN) by a covalent conjugation (esterification via EDC). A first candidate molecule conjugated to a first polynucleotide and a second candidate molecule covalently conjugated to a second polynucleotide are sequentially injected into the flow cell to anchor them on to the nucleic acid scaffold and form a screening nucleic acid scaffold (FIGS. 27 and 28). Then, a force of 2 pN was applied to determine the maximal elongation length. The force was then reduced to 0.01 pN to allow the two proteins to interact. If the two candidate molecules have binding interaction between them, upon increasing the force back to 2 pN, the elongation observed would be less than the full extension of the screening nucleic acid scaffold. If the two candidate molecules have no binding interaction between them or weaker binding interaction between them than the applied force, the nucleic acid scaffold would fully stretch. The two proteins are not believed to have a native interaction. However, when a compound called proteolysis-targeting chimeras (PROTAC) or a molecular glue added in the flowcell, an interaction was observed. Similar results were also observed with the compound CC885 at 10 nM or Thalidomide at InM. Various concentration of compound can be performed to measure the Kon and Koff of interaction in presence of the compound.

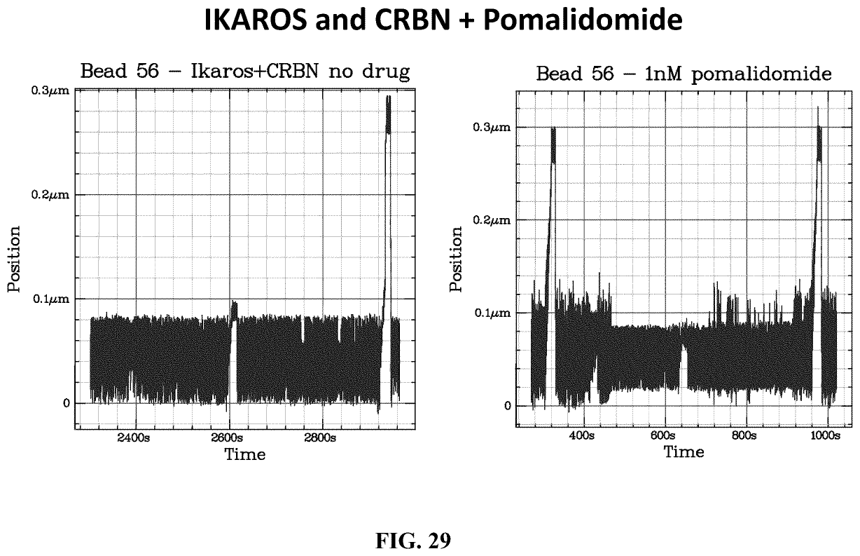

Similar experiment was also performed for another neo-substrate of CRBN E3 ligase, IKZF1 (IKAROS transcription factor) in presence of Pomalidomide at 1 nM, where the interaction was observed (FIG. 29).

### Example 16. Multiplexing Interactions Between One Protein Against Two Other Proteins

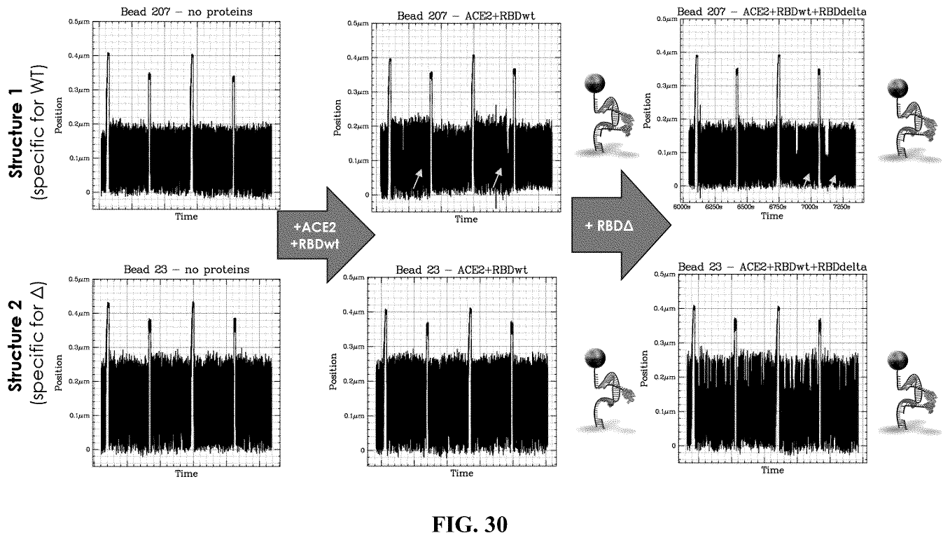

Multiplexing interactions were determined between ACE2 and RBD proteins (wildtype RBD and RBD delta mutant). Briefly, two hairpin nucleic acids were immobilized between a bead and a feature for determining multiplexing interactions: (1) a first hairpin nucleic acid of SEQ ID NO: 48 was used for determining interactions between ACE2 and wildtype RBD; and (2) a second hairpin nucleic acid of SEQ ID NO: 49 was used for determining interactions between ACE2 and RBD delta mutant. Next, three glue sequences were engineered: (1) a first glue sequence of SEQ ID NO: 45 was conjugated to ACE2 protein, the first glue sequence was used for anchoring ACE2 protein to both hairpin nucleic acids; (2) a second glue sequence of SEQ ID NO: 46 was conjugated to wildtype RBD protein, the second glue sequence was used for anchoring wildtype RBD protein to the first hairpin nucleic acid; and (3) a third glue sequence of SEQ ID NO: 47 was conjugated to RBD delta mutant protein, the third glue sequence was used for anchoring RBD delta mutant protein to the second hairpin nucleic acid. Identification of the molecules with the barcode is performed following example 10, preparation of the nucleic acid scaffold is made following example 11. RBD proteins (wildtype RBD protein and RBT delta mutant protein) are then injected separately, and binding interaction and binding kinetics are measured to assess the specificity of anchoring the protein on the specific barcoded molecule. Results of binding interactions between ACE2 and RBD proteins is provided in FIG. 30.

While exemplary embodiments have been shown and described herein, such embodiments are provided by way of example only. Numerous variations, changes, and substitutions are within the scope of the present disclosure. It should be understood that various alternatives to the embodiments described herein may be employed. It is intended that the following claims define the scope of the disclosure and that methods and structures within the scope of these claims and their equivalents be covered thereby.

SEQUENCES

| SEQ | | | |
|---|---|---|---|
| ID | | | |
| NO: | Name | Description | Sequence 5′ to 3′ |
| 1 | | Structure of the polynucleotide | CTTCATGCTGGTCTCAKKKKKKKKKKK |
| | | required to form the Y-shape for | KKKKKKKKXXXXXXXXXXXXXXXCAG |
| | | the hairpin | TGCTCCTTGGCTGATCCG |
| 2 | | Structure of the polynucleotide | attcCGGATCAGTCAAGGAGCACTGYYYY |
| | | required to form the Y-shape for | YYYYYYYYYYYJJJJJJJJJJJJJJJJJJJJTTCCA |
| | | the hairpin | CTTCCTAATCTGTCATCTTCTG |
| 3 | | Sequence recognize by the E. coli | CCwGG |
| | | DCM methylase | |
| 4 | | Example of synthetic barcode | tggtctctgaatgccgccgttatccaggttgaatgaagta |
| | | sequence | tcctggactgCAGATGAATAAACTATGCCAGGCTT |
| | | | TGTGGTGGTGCACACGTCCTTGCGATTA |
| | | | AGTGGACACCAGGCTGCAGTGCCAAAA |
| | | | CGTAACCTGGTTCGCGAGTATATGTTTA |
| | | | CATTCCAGGAGGATAAGATTCAGCGGC |
| | | | ATCCAGGACCTCACTGTTGCGCAGTGA |
| | | | AGAGGTCCTGGATACAAGAAACGAGGT |
| | | | TGTGCCGATTAtacgtagtcgtatcctggtacgtcctgg |
| | | | cgtctggaaggottagagacca |
| 5 | | General structure of the synthetic | Y′Y′Y′Y′Y′Y′Y′Y′Y′Y′Y′Y′Y′Y′Y′CAGT |
| | | oligonucleotide for the Y-shape | GCTCCTTGGCTGATCCG |
| | | require for the space | |
| | | polynucleotide | |
| 6 | | General structure of the synthetic | attcCGGATCAGCCAAGGAGCACTGX′X′X |
| | | oligonucleotide for the Y-shape | ′X′X′X′X′X′X′X′X′X′X′X′X′ |
| | | require for the space | |
| | | polynucleotide | |
| 7 | PS359 | Synthetic loop | P-GCTTGCACTGAGATTTTTCTCAGTGC |
| 8 | PS867 | Splint between hairpin and surface | GTGTCTTTTGGTCTTTCTGGTGCTCTTCG |
| | | oligo | AATCAGAAGATGACAGATTAGGAAGTG |
| | | | G 3′ |
| 9 | PS2416 | Surface oligo | P- |
| | | | ATTCGAAGAGCACCAGAAAGACCAAAA |
| | | | GACACAGTCACAGAT-DBCO |
| 10 | PS3795 | aptamer against SARV-CoV2 RBP | Biotin- |
| | | | UCGGCGAUCUACGCAGCGACAUAUAU |
| | | | GGCGACAUUUGUAAUUCCUGGACCGA |
| | | | UACUUCCGUCAGGACAGAGGUUGCCA |
| | | | UAACAACACUCUCCUCAUCUGUCUCUC |
| | | | C |
| 11 | PS3843 | Y-shape oligo that allows | biotin |
| | | anchoring to the bead | CTTCATGCTGGTCTCAGCTTGATCTCGA |
| | | | AGAGCTACAGCGCCGTACAGACTCAGT |
| | | | GCTCCTTGGCTGATCCG |
| 12 | PS3845 | Y-shape for strand invasion | P- |
| | | | attcCGGATCAGCCAAGGAGCACTGAGTC |
| | | | TGTACGGCGCT |
| 13 | PS3846 | Y-shape for strand invasion | GTCTGTGAATGGATGCAGTGCTCCTTGG |
| | | | CTGATCCG |
| 14 | PS03851 | Y-shape for molecular interaction | attcCGGATCAGTCAAGGAGCACTGCATC |
| | | (with complementary sequence to | CATTCACAGACGTCGCTGCGTaGaTCGCC |
| | | the surface oligo PS625) | GaTTCCACTTCCTAATCTGTCATCTTCTG |
| 15 | PS03852 | Substrate for NHEJ (to be used | tCGGCGAtCtACGCAGCGAC |
| | | with molecular interaction) | GACTGTACATGTCCTCGCACTGGCTCGC |
| | | | TGGATGTGTC-P |
| 16 | PS03853 | Substrate for NHEJ (to be used | GACACATCCAGCGAGCCAGTGCGAGGA |
| | | with molecular interaction) | CATGTACAGTC |
| 17 | PS03854 | Substrate for NHEJ (to be used | GCCATCTGTGGACCCATCCAGGCTTGTG |
| | | with molecular interaction) | AGACTGTAGCTCTTCGAGATCAAGC |
| 18 | PS03855 | Substrate for NHEJ (to be used | AGTCTCACAAGCCTGGATGGGTCCACA |
| | | with molecular interaction) | GATGGC |
| 19 | PS03878 | Oligo on one side of the structure, | tCGGCGAtCtACGCAGCGACGACTGTACA |
| | | which contains a 5mC | TGTCCTCGmCACTGGCTCGCGGTCTCT |
| | | modification and a 4base overhang | |
| | | for ligation with a BsaI and EcoRV | |
| | | site | |
| 20 | PS03879 | Annealed to the oligo to generate | P- |
| | | dsDNA and a 4bp overhang | TATCAGAGACCGCGAGCCAGTGCGAGG |
| | | | ACATGTACAGTC |
| 21 | PS03880 | Oligo on second side of the | P- |
| | | structure, which contains a 5mC | GATATCATCTGTGGACCCATCCAGGCTTG |
| | | modification and a 4base overhang | TGAGACTGTAGCTCTTCGAGATCAAGC |
| | | for ligation with a BsaI and EcoRV | |
| | | site | |
| 22 | PS03881 | Annealed to the oligo to generate | AGTCTCACAAGCCTGGATGGGTCCACA |
| | | dsDNA and a 4 bp overhang | GATGATATCA |
| 23 | PS03887 | Oligo for EDC conjugation with | Amino Modifier |
| | | protein | C6/GCCATCTGTGGACCCATCCAGGCTTG |
| | | | TGAGACTGTAGCTCTTCGAGATCAAGC |
| | | | 3′ |
| 24 | PS03886 | Oligo for Click conjugation with | azide |
| | | protein | C6/GCCATCTGTGGACCCATCCAGGCTTG |
| | | | TGAGACTGTAGCTCTTCGAGATCAAGC |
| 25 | HP131 | Hairpin sequence with the Barcode | CTTCATGCTGGTCTCAGCTTGATCTCGA |
| | | sequence 131 | AGAGCTACAGCGCCGTACAGACTCAGT |
| | | | GCTCCTTGGCTGATCCGgaatgccgccgttatcca |
| | | | ggttgaatgaagtatcctggactgtgcgggacccagaacaac |
| | | | cctggtggctattctaatacagcctggaggcgtaacacctgg |
| | | | tgcatggacttcaggctgtttcagttccttaagcacgacagc |
| | | | ctcgtagtcagtagcctggtacgatcatactaccctgggctg |
| | | | aagttctttcgtagtttccactgccgtattgcagcatgatac |
| | | | cttgtcagaagccttacgtagtcgtatcctggtacgtcctgg |
| | | | cgtctggaaggcttGCTTGCACTGAGATTTTTCTCAGTGCaa |
| | | | gccttccagacgccaggacgtaccaggatacgactacgtaag |
| | | | gcttctgacaaggtatcatgctgcaatacggcagtggaaact |
| | | | acgaaagaacttcagcccagggtagtatgatcgtaccaggct |
| | | | actgactacgaggctgtcgtgcttaaggaactgaaacagcct |
| | | | gaagtccatgcaccaggtgttacgcctccaggctgtattaga |
| | | | atagccaccagggttgttctgggtcccgcacagtccaggata |
| | | | cttcattcaacctggataacggcggcattcCGGATCAGTCAA |
| | | | GGAGCACTGCATCCATTCACAGACGTCGCTGCGTAGAT |
| | | | CGCCGATTCCACTTCCTAATCTGTCATCT |
| | | | TCTG |
| 26 | HP133 | Hairpin sequence with the Barcode | CTTCATGCTGGTCTCAGCTTGATCTCGA |
| | | sequence 133 | AGAGCTACAGCGCCGTACAGACTCAGT |
| | | | GCTCCTTGGCTGATCCGgaatgccgccgttatcca |
| | | | ggttgaatgaagtatcctggactgtgctgtcgggttaagcca |
| | | | cgtattaaccaggttaacatagtgccattatctctttatcag |
| | | | ggcacgctctaaactccagggcttagggacgcaaagccaagg |
| | | | accggcttggccaggtatcggtaaatttgcctcagatgcgat |
| | | | ttgcaaattgaccaggttacgccggcataggctaggttgaga |
| | | | tttggccaggctcgtacgtagtcgtatcctggtacgtcctgg |
| | | | cgtctggaaggcttGCTTGCACTGAGATTTTTCTCAGTGCaa |
| | | | gccttccagacgccaggacgtaccaggatacgactacgtacg |
| | | | agcctggccaaatctcaacctagcctatgccggcgtaacctg |
| | | | gtcaatttgcaaatcgcatctgaggcaaatttaccgatacct |
| | | | ggccaagccggtccttggctttgcgtccctaagccctggagt |
| | | | ttagagcgtgccctgataaagagataatggcactatgttaac |
| | | | ctggttaatacgtggcttaacccgacagcacagtccaggata |
| | | | cttcattcaacctggataacggcggcattcCGGATCAGTCAA |
| | | | GGAGCACTGCATCCATTCACAGACGTCGCTGCGTAGATCGC |
| | | | CGATTCCACTTCCTAATCTGTCATCTTCT |
| | | | G |
| 27 | HP134 | Hairpin sequence with the Barcode | CTTCATGCTGGTCTCAGCTTGATCTCGA |
| | | sequence 134 | AGAGCTACAGCGCCGTACAGACTCAGT |
| | | | GCTCCTTGGCTGATCCGgaatgccgccgttatcca |
| | | | ggttgaatgaagtatcctggactgaggacagcccaggcagtc |
| | | | gcaatacagaaattagactaactccgtgggccaccgacgttc |
| | | | atctggccaagtctccgccaggacttgtgttgatgtcaaggt |
| | | | tgcacgtgaaatccatccaggccaaccagcgggcctgtggca |
| | | | tattcacggtggattcccaggtaacaccgaatatcgcctggg |
| | | | tgccctttgctacttacgtagtcgtatcctggtacgtcctgg |
| | | | cgtctggaaggcttGCTTGCACTGAGATTTTTCTCAGTGCaa |
| | | | gccttccagacgccaggacgtaccaggatacgactacgtaag |
| | | | tagcaaagggcacccaggcgatattcggtgttacctgggaat |
| | | | ccaccgtgaatatgccacaggcccgctggttggcctggatgg |
| | | | atttcacgtgcaaccttgacatcaacacaagtcctggcggag |
| | | | acttggccagatgaacgtcggtggcccacggagttagtctaa |
| | | | tttctgtattgcgactgcctgggctgtcctcagtccaggata |
| | | | cttcattcaacctggataacggcggcattcCGGATCAGTCAA |
| | | | GGAGCACTGCATCCATTCACAGACGTCGCTGCGTAGAT |
| | | | CGCCGATTCCACTTCCTAATCTGTCATCT |
| | | | TCTG |
| 28 | HP135 | Hairpin sequence with the Barcode | CTTCATGCTGGTCTCAGCTTGATCTCGA |
| | | sequence 135 | AGAGCTACAGCGCCGTACAGACTCAGT |
| | | | GCTCCTTGGCTGATCCGgaatgccgccgttatcca |
| | | | ggttgaatgaagtatcctggactgaggacagcccaggcagtc |
| | | | gcaatacagaaattagactaactccgtgggccaccgacgttc |
| | | | atctggccaagtctccgccaggacttgtgttgatgtcaaggt |
| | | | tgcacgtgaaatccatccaggccaaccagcgggcctgtggca |
| | | | tattcacggtggattcccaggtaacaccgaatatcgcctggg |
| | | | tgccctttgctacttacgtagtcgtatcctggtacgtcctgg |
| | | | cgtctggaaggcttGCTTGCACTGAGATTTTTCTCAGTGCaa |
| | | | gccttccagacgccaggacgtaccaggatacgactacgtaag |
| | | | tagcaaagggcacccaggcgatattcggtgttacctgggaat |
| | | | ccaccgtgaatatgccacaggcccgctggttggcctggatgg |
| | | | atttcacgtgcaaccttgacatcaacacaagtcctggcggag |
| | | | acttggccagatgaacgtcggtggcccacggagttagtctaa |
| | | | tttctgtattgcgactgcctgggctgtcctcagtccaggata |
| | | | cttcattcaacctggataacggcggcattcCGGATCAGTCAA |
| | | | GGAGCACTGCATCCATTCACAGACGTCGCTGCGTAGAT |
| | | | CGCCGATTCCACTTCCTAATCTGTCATCT |
| | | | TCTG |
| 29 | HP136 | Hairpin sequence with the Barcode | CTTCATGCTGGTCTCAGCTTGATCTCGA |
| | | sequence 136 | AGAGCTACAGCGCCGTACAGACTCAGT |
| | | | GCTCCTTGGCTGATCCGgaatgccgccgttatcca |
| | | | ggttgaatgaagtatcctggactgcctgggcctatgtaacag |
| | | | ggtgagcgcattacggcccggtatgcccagggccactacatg |
| | | | agcacgatgctaacagtgacacctggtagcttagcacatcca |
| | | | gagtgtgtagcgaccgccagggagtccgttggataggtgttg |
| | | | attgattagatcctggccgaatatggaaagtcggtaggtatg |
| | | | attgttctcgcccgtacgtagtcgtatcctggtacgtcctgg |
| | | | cgtctggaaggcttGCTTGCACTGAGATTTTTCTCAGTGCaa |
| | | | gccttccagacgccaggacgtaccaggatacgactacgtacg |
| | | | ggcgagaacaatcatacctaccgactttccatattcggccag |
| | | | gatctaatcaatcaacacctatccaacggactccctggcggt |
| | | | cgctacacactctggatgtgctaagctaccaggtgtcactgt |
| | | | tagcatcgtgctcatgtagtggccctgggcataccgggccgt |
| | | | aatgcgctcaccctgttacataggcccaggcagtccaggata |
| | | | cttcattcaacctggataacggcggcattcCGGATCAGTCAA |
| | | | GGAGCACTGCATCCATTCACAGACGTCGCTGCGTAGAT |
| | | | CGCCGATTCCACTTCCTAATCTGTCATCT |
| | | | TCTG |
| 30 | HP138 | Hairpin sequence with the Barcode | CTTCATGCTGGTCTCAGCTTGATCTCGA |
| | | sequence 138 | AGAGCTACAGCGCCGTACAGACTCAGT |
| | | | GCTCCTTGGCTGATCCGgaatgccgccgttatcca |
| | | | ggttgaatgaagtatcctggactgatgattccatctccgtta |
| | | | tccaggcgactttgtgcgttcatatatgatgagtaatccacg |
| | | | agtatattaatagatcctggcagatcctggtccagggaccct |
| | | | gaccgcgcgggccaggaacaggcaagctggtcaagcctggtg |
| | | | ccgaacattgttacctagttaaaggccagtagtccctgccca |
| | | | ataaccttccaccatacgtagtcgtatcctggtacgtcctgg |
| | | | cgtctggaaggcGCTTGCACTGAGATTTTTCTCAGTGCgcct |
| | | | tccagacgccaggacgtaccaggatacgactacgtatggtgg |
| | | | aaggttattgggcagggactactggcctttaactaggtaaca |
| | | | atgttcggcaccaggcttgaccagcttgcctgttcctggccc |
| | | | gcgcggtcagggtccctggaccaggatctgccaggatctatt |
| | | | aatatactcgtggattactcatcatatatgaacgcacaaagt |
| | | | cgcctggataacggagatggaatcatcagtccaggatacttc |
| | | | attcaacctggataacggcggcattccCGGATCAGTCAAGGA |
| | | | GCACTGCATCCATTCACAGACGTCGCTGCGTAGATCGCC |
| | | | GATTCCACTTCCTAATCTGTCATCTTCTG |
| 31 | HP139 | Hairpin sequence with the Barcode | CTTCATGCTGGTCTCAGCTTGATCTCGA |
| | | sequence 139 | AGAGCTACAGCGCCGTACAGACTCAGT |
| | | | GCTCCTTGGCTGATCCGgaatgccgccgttatcca |
| | | | ggttgaatgaagtatcctggactgcaagttctcagtccctgg |
| | | | cgtactgttcagacctccgcagccgttgccaggtgggagtgc |
| | | | aaacaccacatacaacacgtttcggccttattccagcctggt |
| | | | caacgaccagcgcctggttgtcagcgcaattccttatacccg |
| | | | ccacctggcggctagtagacaggatattattagggccacggg |
| | | | ccgtaagtccgtcgtacgtagtcgtatcctggtacgtcctgg |
| | | | cgtctggaaggcttGCTTGCACTGAGATTTTTCTCAGTGCaa |
| | | | gccttccagacgccaggacgtaccaggatacgactacgtacg |
| | | | acggacttacggcccgtggccctaataatatcctgtctacta |
| | | | gccgccaggtggcgggtataaggaattgcgctgacaaccagg |
| | | | cgctggtcgttgaccaggctggaataaggccgaaacgtgttg |
| | | | tatgtggtgtttgcactcccacctggcaacggctgcggaggt |
| | | | ctgaacagtacgccagggactgagaacttgcagtccaggata |
| | | | cttcattcaacctggataacggcggcattcCGGATCAGTCAA |
| | | | GGAGCACTGCATCCATTCACAGACGTCGCTGCGTAGA |
| | | | TCGCCGATTCCACTTCCTAATCTGTCATC |
| | | | TTCTG |
| 32 | HP140 | Hairpin sequence with the Barcode | CTTCATGCTGGTCTCAGCTTGATCTCGA |
| | | sequence 140 | AGAGCTACAGCGCCGTACAGACTCAGT |
| | | | GCTCCTTGGCTGATCCGgaatgccgccgttatcca |
| | | | ggttgaatgaagtatcctggactgatcgcgaaagatttcagg |
| | | | aacacctccctggtggaacttagtctatgccatagttaagct |
| | | | aaattgccagggcagtcgtgactgcgccgtaggaacaagtcc |
| | | | ctccaagtgtgggacgcacctggtcgcaaagcacgcctggag |
| | | | gctactatggtttagcccagggcgttacacactaatccgcct |
| | | | ttgttgtgtagttatacgtagtcgtatcctggtacgtcctgg |
| | | | cgtctggaaggcttGCTTGCACTGAGATTTTTCTCAGTGCaa |
| | | | gccttccagacgccaggacgtaccaggatacgactacgtata |
| | | | actacacaacaaaggcggattagtgtgtaacgccctgggcta |
| | | | aaccatagtagcctccaggcgtgctttgcgaccaggtgcgtc |
| | | | ccacacttggagggacttgttcctacggcgcagtcacgactg |
| | | | ccctggcaatttagcttaactatggcatagactaagttccac |
| | | | cagggaggtgttcctgaaatctttcgcgatcagtccaggata |
| | | | cttcattcaacctggataacggcggcattcCGGATCAGTCAA |
| | | | GGAGCACTGCATCCATTCACAGACGTCGCTGCGTAGATCGC |
| | | | CGATTCCACTTCCTAATCTGTCATCTTCT |
| | | | G |
| 33 | SI140 | Space polynucleotide hairpin | GTCTGTGAATGGATGCAGTGCTCCTTGG |
| | | specific for HP140 | CTGATCCGgaatgccgccgttatccaggttgaatgaagtat |
| | | | cctggactgatcgcgaaagatttcaggaacacctccctggtg |
| | | | gaacttagtctatgccatagttaagctaaattgccagggcag |
| | | | tcgtgactgcgccgtaggaacaagtccctccaagtgtgggac |
| | | | gcacctggtcgcaaagcacgcctggaggctactatggtttag |
| | | | cccagggcgttacacactaatccgcctttgttgtgtagttat |
| | | | acgtagtcgtatcctggtacgtcctggcgtctggaaggcttG |
| | | | CTTGCACTGAGAAAAAACTCAGTGCaagccttccagacgcca |
| | | | ggacgtaccaggatacgactacgtataactacacaacaaagg |
| | | | cggattagtgtgtaacgccctgggctaaaccatagtagcctc |
| | | | caggcgtgctttgcgaccaggtgcgtcccacacttggaggga |
| | | | cttgttcctacggcgcagtcacgactgccctggcaatttagc |
| | | | ttaactatggcatagactaagttccaccagggaggtgttcct |
| | | | gaaatctttcgcgatcagtccaggatacttcattcaacctgg |
| | | | ataacggcggcattcCGGATCAGCCAAGGAGCACTGAGTCTG |
| | | | TACGGCGCT |
| 34 | | PS03919 | 5′ |
| | | | TCGGCGAtCtACGCAGCGACGCCATCTGT |
| | | | GGACCCATCCAGGCTTGTGAGACT C6- |
| | | | NH2 |

| | | | |
|---|---|---|---|
| 35 | | Seqeunce of the universal strand | GGTCTCTGAATctgagttgatatgtccaggtatttgtccta |
| | | invasion fragment and the template | ttatccaggttctacatggttctgtgaacaaggttgcacaag |
| | | that serve to generate all the | gttacgcatctcaaggttactcaaggtcaatgattgacaagc |
| | | barcodes | atggttctacatggtgtgtacatgcaaggtagtacctatgac |
| | | | tacatggtgcaacatggttcttgcttgtctatcaaggtcaca |
| | | | tatgtcaaggttacattgtctgttcacatggtcactcatatc |
| | | | aaggtactatagacgagttgataaccaggtgtatccaggttt |
| | | | caagcattgaGCTTAGAGACC |
| 36 | | PS4092 | biotin |
| | | | CTTCATGCGCTCTGAGTCTCTGCGAGCG |
| | | | CACGTGGCGACGGCACGACGCCTTTTG |
| | | | GCGTCGTGCCGTCGCCACGTGCGCTCGC |
| | | | ACGTCACAGCTGAAGCGCCGTACAGAC |
| | | | TCAGTGCTCCTTGGCTGATCCG |
| 37 | | PS4093 | /5Phos/ATTCCGGATCAGCCAAGGAGCAC |
| | | | TGCATCCATTCACAGACGTCGCTGACTG |
| | | | TTGCGAGCGCACGTGGCGACGGCACGA |
| | | | CGCCTTTTGGCGTCGTGCCGTCGCCACG |
| | | | TGCGCTCGCACAGACTCGCTCGATCTGT |
| | | | CATCTTCTGT |
| 38 | SI | Universal spacer polynucleotide | GTCTGTGAATGGATGCAGTGCTCCTTGG |
| | | hairpin sequence | CTGATCCGGAATctgagttgatatgtccaggtatttgtcc |
| | | | tattatccaggttctacatggatctgtgaacaaggttgcaca |
| | | | aggttacgcatctcaaggatactcaaggtcaatgattgacaa |
| | | | gcatggttctacatggagtgtacatgcaaggtagtacctatg |
| | | | actacatggagcaacatggttcttgcttgtctatcaaggact |
| | | | catatgtcaaggttacattgtctgttcacatggactctcata |
| | | | tcaaggtactatagacgagttgataaccaggtgtatccagga |
| | | | ttcaagcattgaGCTTGCACTGAGAaaaaTCTCAGTGCAAGC |
| | | | tcaatgcttgaatcctggatacacctggttatcaactcgtct |
| | | | atagtaccttgatatgagagtccatgtgaacagacaatgtaa |
| | | | ccttgacatatgagtccttgatagacaagcaagaaccatgtt |
| | | | gctccatgtagtcataggtactaccttgcatgtacactccat |
| | | | gtagaaccatgcttgtcaatcattgaccttgagtatccttga |
| | | | gatgcgtaaccttgtgcaaccttgttcacagatccatgtaga |
| | | | acctggataataggacaaatacctggacatatcaactcagAT |
| | | | TCCGGATCAGCCAAGGAGCACTGAGTCTGTACGGCGCT |
| 39 | Uni- | Sequence of the hairpin produced | biotin |
| | versal | from the universal sequence | CTTCATGCGCTCTGAGTCTCTGCGAGCG |
| | HP | | CACGTGGCGACGGCACGACGCCTTTTG |
| | | | GCGTCGTGCCGTCGCCACGTGCGCTCGC |
| | | | ACGTCACAGCTGAAGCGCCGTACAGAC |
| | | | TCAGTGCTCCTTGGCTGATCCGGAATctga |
| | | | gttgatatgtccaggtatttgtcctattatccaggttctaca |
| | | | tggatctgtgaacaaggttgcacaaggttacgcatctcaagg |
| | | | atactcaaggtcaatgattgacaagcatggttctacatggag |
| | | | tgtacatgcaaggtagtacctatgactacatggagcaacatg |
| | | | gttcttgcttgtctatcaaggactcatatgtcaaggttacat |
| | | | tgtctgttcacatggactctcatatcaaggtactatagacga |
| | | | gttgataaccaggtgtatccaggattcaagcattgaGCTTGC |
| | | | TTGCACTGAGATTTTTCTCAGTGCAAGCAAGCtcaatgcttg |
| | | | aatcctggatacacctggttatcaactcgtctatagtacctt |
| | | | gatatgagagtccatgtgaacagacaatgtaaccttgacata |
| | | | tgagtccttgatagacaagcaagaaccatgttgctccatgta |
| | | | gtcataggtactaccttgcatgtacactccatgtagaaccat |
| | | | gcttgtcaatcattgaccttgagtatccttgagatgcgtaac |
| | | | cttgtgcaaccttgttcacagatccatgtag |
| | | | aacctggataataggacaaatacctggacatatcaactcagA |
| | | | TTCCGGATCAGCCAAGGAGCACTGCATCC |
| | | | ATTCACAGACGTCGCTGACTGTTGCGAG |
| | | | CGCACGTGGCGACGGCACGACGCCTTT |
| | | | TGGCGTCGTGCCGTCGCCACGTGCGCTC |
| | | | GCACAGACTCGCTCGATCTGTCATCTTC |
| | | | TGT |
| 40 | BC1 | Sequence of the hairpin produced | biotin |
| | | from the barcode 1 (BC1) | CTTCATGCGCTCTGAGTCTCTGCGAGCG |
| | | sequence | CACGTGGCGACGGCACGACGCCTTTTG |
| | | | GCGTCGTGCCGTCGCCACGTGCGCTCGC |
| | | | ACGTCACAGCTGAAGCGCCGTACAGAC |
| | | | TCAGTGCTCCTTGGCTGATCCGgaatctgag |
| | | | ttgatatgtccaggtatttgtcctattatccaggttctacct |
| | | | ggatctgtgaaccaggttgcaccaggttacgcatctcaagga |
| | | | tactcaaggtcaatgattgacaagcatggttctacatggagt |
| | | | gtacatgcaaggtagtacctatgactacatggagcaacatgg |
| | | | ttcttgcttgtctatcaaggactcatatgtcaaggttacatt |
| | | | gtctgttcacatggactctcatatcaaggtactatagacgag |
| | | | ttgataaccaggtgtatccaggattcaagcattgaGCTTGCA |
| | | | CTGAGATTTTTCTCAGTGCAAGCtcaatgcttga |
| | | | atcctggatacacctggttatcaactogtctatagtaccttg |
| | | | atatgagagtccatgtgaacagacaatgtaaccttgacatat |
| | | | gagtccttgatagacaagcaagaaccatgttgctccatgtag |
| | | | tcataggtactaccttgcatgtacactccatgtagaaccatg |
| | | | cttgtcaatcattgaccttgagtatccttgagatgcgtaacc |
| | | | tggtgcaacctggttcacagatccaggtagaacctggataat |
| | | | aggacaaatacctggacatatcaactcagattcCGGATCAGC |
| | | | CAAGGAGCACTGCATCCATTCACAGACGTCGCTGACTGTTG |
| | | | CGAGCGCACGTGGCGACGGCACGACGC |
| | | | CTTTTGGCGTCGTGCCGTCGCCACGTGC |
| | | | GCTCGCACAGACTCGCTCGATCTGTCAT |
| | | | CTTCTGT |
| 41 | BC2 | Sequence of the hairpin produced | biotin |
| | | from the barcode 2 (BC2) | CTTCATGCGCTCTGAGTCTCTGCGAGCG |
| | | sequence | CACGTGGCGACGGCACGACGCCTTTTG |
| | | | GCGTCGTGCCGTCGCCACGTGCGCTCGC |
| | | | ACGTCACAGCTGAAGCGCCGTACAGAC |
| | | | TCAGTGCTCCTTGGCTGATCCGgaatctgag |
| | | | ttgatatgtccaggtatttgtcctattatccaggttctacct |
| | | | ggatctgtgaacaaggttgcacaaggttacgcatctccagga |
| | | | tactcaaggtcaatgattgacaagcatggttctacctggagt |
| | | | gtacatgcaaggtagtacctatgactacatggagcaacctgg |
| | | | ttcttgcttgtctatcaaggactcatatgtcaaggttacatt |
| | | | gtctgttcacatggactctcatatcaaggtactatagacgag |
| | | | ttgataaccaggtgtatccaggattcaagcattgaGCTTGCA |
| | | | CTGAGATTTTTCTCAGTGCAAGCtcaatgcttga |
| | | | atcctggatacacctggttatcaactcgtctatagtaccttg |
| | | | atatgagagtccatgtgaacagacaatgtaaccttgacatat |
| | | | gagtccttgatagacaagcaagaaccaggttgctccatgtag |
| | | | tcataggtactaccttgcatgtacactccaggtagaaccatg |
| | | | cttgtcaatcattgaccttgagtatcctggagatgcgtaacc |
| | | | ttgtgcaaccttgttcacagatccaggtagaacctggataat |
| | | | aggacaaatacctggacatatcaactcagattcCGGATCAGC |
| | | | CAAGGAGCACTGCATCCATTCACAGACGTCGCTGACTGTT |
| | | | GCGAGCGCACGTGGCGACGGCACGACG |
| | | | CCTTTTGGCGTCGTGCCGTCGCCACGTG |
| | | | CGCTCGCACAGACTCGCTCGATCTGTCA |
| | | | TCTTCTGT |
| 42 | BC3 | Sequence of the hairpin produced | biotin |
| | | from the barcode 3 (BC3) | CTTCATGCGCTCTGAGTCTCTGCGAGCG |
| | | sequence | CACGTGGCGACGGCACGACGCCTTTTG |
| | | | GCGTCGTGCCGTCGCCACGTGCGCTCGC |
| | | | ACGTCACAGCTGAAGCGCCGTACAGAC |
| | | | TCAGTGCTCCTTGGCTGATCCGgaatctgag |
| | | | ttgatatgtccaggtatttgtcctattatccaggttctacct |
| | | | ggatctgtgaaccaggttgcacaaggttacgcatctccagga |
| | | | tactccaggtcaatgattgacaagcctggttctacatggagt |
| | | | gtacatgcaaggtagtacctatgactacatggagcaacatgg |
| | | | ttcttgcttgtctatcaaggactcatatgtcaaggttacatt |
| | | | gtctgttcacatggactctcatatcaaggtactatagacgag |
| | | | ttgataaccaggtgtatccaggattcaagcattgaGCTTGCA |
| | | | CTGAGATTTTTCTCAGTGCAAGCtcaatgcttga |
| | | | atcctggatacacctggttatcaactcgtctatagtaccttg |
| | | | atatgagagtccatgtgaacagacaatgtaaccttgacatat |
| | | | gagtccttgatagacaagcaagaaccatgttgctccatgtag |
| | | | tcataggtactaccttgcatgtacactccatgtagaaccagg |
| | | | cttgtcaatcattgacctggagtatcctggagatgcgtaacc |
| | | | ttgtgcaacctggttcacagatccaggtagaacctggataat |
| | | | aggacaaatacctggacatatcaactcagattcCGGATCAGC |
| | | | CAAGGAGCACTGCATCCATTCACAGACGTCGCTGACTGTT |
| | | | GCGAGCGCACGTGGCGACGGCACGACG |
| | | | CCTTTTGGCGTCGTGCCGTCGCCACGTG |
| | | | CGCTCGCACAGACTCGCTCGATCTGTCA |
| | | | TCTTCTGT |
| 43 | BC4 | Sequence of the hairpin produced | biotin |
| | | from the barcode 4 (BC4) | CTTCATGCGCTCTGAGTCTCTGCGAGCG |
| | | sequence | CACGTGGCGACGGCACGACGCCTTTTG |
| | | | GCGTCGTGCCGTCGCCACGTGCGCTCGC |
| | | | ACGTCACAGCTGAAGCGCCGTACAGAC |
| | | | TCAGTGCTCCTTGGCTGATCCGgaatctgag |
| | | | ttgatatgtccaggtatttgtcctattatccaggttctacct |
| | | | ggatctgtgaaccaggttgcacaaggttacgcatctccagga |
| | | | tactcaaggtcaatgattgacaagcatggttctacctggagt |
| | | | gtacatgcaaggtagtacctatgactacatggagcaacatgg |
| | | | ttcttgcttgtctatccaggactcatatgtcaaggttacatt |
| | | | gtctgttcacctggactctcatatcaaggtactatagacgag |
| | | | ttgataaccaggtgtatccaggattcaagcattgaGCTTGCA |
| | | | CTGAGATTTTTCTCAGTGCAAGCtcaatgcttga |
| | | | atcctggatacacctggttatcaactcgtctatagtaccttg |
| | | | atatgagagtccaggtgaacagacaatgtaaccttgacatat |
| | | | gagtcctggatagacaagcaagaaccatgttgctccatgtag |
| | | | tcataggtactaccttgcatgtacactccaggtagaaccatg |
| | | | cttgtcaatcattgaccttgagtatcctggagatgcgtaacc |
| | | | ttgtgcaacctggttcacagatccaggtagaacctggataat |
| | | | aggacaaatacctggacatatcaactcagattcCGGATCAGC |
| | | | CAAGGAGCACTGCATCCATTCACAGACGTCGCTGACTGTT |
| | | | GCGAGCGCACGTGGCGACGGCACGACG |
| | | | CCTTTTGGCGTCGTGCCGTCGCCACGTG |
| | | | CGCTCGCACAGACTCGCTCGATCTGTCA |
| | | | TCTTCTGT |
| 44 | BC5 | Sequence of the hairpin produced | biotin |
| | | from the barcode 5 (BC5) | CTTCATGCGCTCTGAGTCTCTGCGAGCG |
| | | sequence | CACGTGGCGACGGCACGACGCCTTTTG |
| | | | GCGTCGTGCCGTCGCCACGTGCGCTCGC |
| | | | ACGTCACAGCTGAAGCGCCGTACAGAC |
| | | | TCAGTGCTCCTTGGCTGATCCGgaatctgag |
| | | | ttgatatgtccaggtatttgtcctattatccaggttctacat |
| | | | ggatctgtgaacaaggttgcacaaggttacgcatctcaagga |
| | | | tactcaaggtcaatgattgacaagcatggttctacctggagt |
| | | | gtacatgccaggtagtacctatgactacctggagcaacatgg |
| | | | ttcttgcttgtctatcaaggactcatatgtcaaggttacatt |
| | | | gtctgttcacatggactctcatatcaaggtactatagacgag |
| | | | ttgataaccaggtgtatccaggattcaagcattgaGCTTGCA |
| | | | CTGAGATTTTTCTCAGTGCAAGCtcaatgcttga |
| | | | atcctggatacacctggttatcaactcgtctatagtaccttg |
| | | | atatgagagtccatgtgaacagacaatgtaaccttgacatat |
| | | | gagtccttgatagacaagcaagaaccatgttgctccaggtag |
| | | | tcataggtactacctggcatgtacactccaggtagaaccatg |
| | | | cttgtcaatcattgaccttgagtatccttgagatgcgtaacc |
| | | | ttgtgcaaccttgttcacagatccatgtagaacctggataat |
| | | | aggacaaatacctggacatatcaactcagattcCGGATCAGC |
| | | | CAAGGAGCACTGCATCCATTCACAGACGTCGCTGACTGTTG |
| | | | CGAGCGCACGTGGCGACGGCACGACGC |
| | | | CTTTTGGCGTCGTGCCGTCGCCACGTGC |
| | | | GCTCGCACAGACTCGCTCGATCTGTCAT |
| | | | CTTCTGT |
| 45 | PS3924 | Oligonucleotide conjugated to | tCGGCGAtCtACGCAGCGACGCCATCT |
| | | ACE2 protein for the multiplexing | GTGGACCCATCCAGGCTTGTGAGAC |
| | | experiment | T-Azide |
| 46 | PS3992 | Oligonucleotide conjugated to | Azide- |
| | | RBD delta mutant protein for the | GCCATCTGTGGACCCATCCAGGCTTGTG |
| | | multiplexing experiment | AGACTGTCACACGATATCTCAGCTA |
| 47 | PS3991 | To create a second Y-shape for the | biotin |
| | | multiplexing experiment to anchor | CTTCATGCTGGTCTCATAGCTGAGATATC |
| | | RBD Delta. Used with PS3851 | GTGTGACAGCGCCGTACAGACTCAGTG |
| | | | CTCCTTGGCTGATCCG |
| 48 | HP155 | Hairpin used for the multiplexing, | Biotin- |
| | | specific for the RBD wild type | cttcatgctggtctcagcttgatctcgaagagctacagcgcc |
| | | protein (Sequence 105 | gtacagactcagtgctccttggctgatccgGAATGCCGCCGT |
| | | complementary to the | TATCCAGGTTGAATGAAGTATCCTGGAC |
| | | oligonucleotide PS3886 and | TGATCGCGAAAGATTTCAGGAACACCTC |
| | | sequence 104 to the | CCTGGTGGAACTTAGTCTATGCCATAGTT |
| | | | AAGCTAAATTGCAAGGGCAGTCGTGACT |
| | | | GCGCCGTAGGAACAAGTCCCTCCAAGT |
| | | | GTGGGACGCACATGGTCGCAAAGCACG |
| | | | CATGGAGGCTACTATGGTTTAGCCCAGG |
| | | | GCGTTACACACTAATCCGCCTTTGTTGT |
| | | | GTAGTTATACGTAGTCGTATCCTGGTACG |
| | | | TCCTGGCGTCTGGAAGGCTTGCACTGAG |
| | | | ATTTTTCTCAGTGCAAGCCTTCCAGACG |
| | | | CCAGGACGTACCAGGATACGACTACGTA |
| | | | TAACTACACAACAAAGGCGGATTAGTGT |
| | | | GTAACGCCCTGGGCTAAACCATAGTAGC |
| | | | CTCCATGCGTGCTTTGCGACCATGTGCG |
| | | | TCCCACACTTGGAGGGACTTGTTCCTAC |
| | | | GGCGCAGTCACGACTGCCCTTGCAATTT |
| | | | AGCTTAACTATGGCATAGACTAAGTTCC |
| | | | ACCAGGGAGGTGTTCCTGAAATCTTTCG |
| | | | CGATCAGTCCAGGATACTTCATTCAACC |
| | | | TGGATAACGGCGGCATTCcggatcagccaagga |
| | | | gcactgCATCCATTCACAGACGTCGCTGCG |
| | | | TaGaTCGCCGaTTCCACTTCCTAATCTGTC |
| | | | ATCTTCTG |
| 49 | HP153 | oligonucleotide PS3924) | biotin |
| | | Hairpin used for the multiplexing, | cttcatgctggtctcatagctgagatatcgtgtgacagogcc |
| | | specific for the RBD delta mutant | gtacagactcagtgctccttggctgatccgGAATGCCGCCGT |
| | | protein (Sequence 105 | TATCCAGGTTGAATGAAGTATCCTGGAC |
| | | complementary to the | TGATCGCGAAAGATTTCAGGAACACCTC |
| | | oligonucleotide PS3992 and | CATGGTGGAACTTAGTCTATGCCATAGTT |
| | | sequence 104 to the | AAGCTAAATTGCAAGGGCAGTCGTGACT |
| | | oligonucleotide PS3924) | GCGCCGTAGGAACAAGTCCCTCCAAGT |
| | | | GTGGGACGCACCTGGTCGCAAAGCACG |
| | | | CCTGGAGGCTACTATGGTTTAGCCCAGG |
| | | | GCGTTACACACTAATCCGCCTTTGTTGT |
| | | | GTAGTTATACGTAGTCGTATCCTGGTACG |
| | | | TCCTGGCGTCTGGAAGGCTTGCACTGAG |
| | | | ATTTTTCTCAGTGCAAGCCTTCCAGACG |
| | | | CCAGGACGTACCAGGATACGACTACGTA |
| | | | TAACTACACAACAAAGGCGGATTAGTGT |
| | | | GTAACGCCCTGGGCTAAACCATAGTAGC |
| | | | CTCCAGGCGTGCTTTGCGACCAGGTGCG |
| | | | TCCCACACTTGGAGGGACTTGTTCCTAC |
| | | | GGCGCAGTCACGACTGCCCTTGCAATTT |
| | | | AGCTTAACTATGGCATAGACTAAGTTCC |
| | | | ACCATGGAGGTGTTCCTGAAATCTTTCG |
| | | | CGATCAGTCCAGGATACTTCATTCAACC |
| | | | TGGATAACGGCGGCATTCoggatcagccaagga |
| | | | gcactgCATCCATTCACAGACGTCGCTGCG |
| | | | TaGaTCGCCGaTTCCACTTCCTAATCTGTC |
| | | | ATCTTCTG |
| 50 | | Prototype sequence that | XXXXXXXXXXXXTGCGAGCGCACGTGG |
| | | corresponds to sequence 104 and | CGACGGCACGACGCCttttGGCGTCGTGCC |
| | | 105 in figure 1. The Xs represent | GTCGCCACGTGCGCTCGCAXXXXXXXX |
| | | sequence required for the | XXXX |
| | | specificity | |
| 51 | | Prototype sequence of the | M- |
| | | oligonucleotide covalently | GTGACTGAGTCAGAGtttYYYYYYYYYY |
| | | attached to the protein. The M | YYTGCGAGCGCACGTGGCGACGGCACG |
| | | represents the modification | ACGCCaaaaGGCGTCGTGCCGTCGCCAC |
| | | required for the chemistry to | GTGCGCTCGCAYYYYYYYYYYYY |
| | | attach the oligo on the protein | |
| 52 | | Prototype sequence of the | YYYYYYYYYYYYTGCGAGCGCACGTGG |
| | | oligonucleotide covalently | CGACGGCACGACGCCaaaaGGCGTCGTG |
| | | attached to the protein. The M | CCGTCGCCACGTGCGCTCGCAYYYYYY |
| | | represents the modification | YYYYYYtttGTGACTGAGTCAGAG-M |
| | | required for the chemistry to attach | |
| | | the oligo on the protein | |
| 53 | | Example of synthetic barcode | GGTCTCTGAATctgagttgatatgtccaggtatttgtccta |
| | | sequence | ttatccaggttctacatggttctgtgaacaaggttgcacacg |
| | | | gttacgcatctcaaggttactcaaggtcaatgattgacaagc |
| | | | atggttctacatggtgtgtacatgcaaggtagtacctatgac |
| | | | tacatggtgcaacatggttcttgcttgtctatcaaggtcaca |
| | | | tatgtcaaggttacattgtctgttcacatggtcactcatatc |
| | | | aaggtactatagacgagttgataaccaggtgtatccaggttt |
| | | | caagcattgaGCTTAGAGACC |
| 54 | | | CawGG |

1. A nucleic acid scaffold for determining a binding interaction between a first candidate molecule and a second candidate molecule, wherein the nucleic acid scaffold comprises:
(a) a contiguous polynucleotide sequence comprising:
(i) a first end that is attached to a bead, wherein the first end comprises a first molecule binding sequence,
(ii) a second end that is attached to a bottom surface of a device, wherein the second end comprises a second molecule binding sequence, and
(iii) an intermediate portion between the first molecule binding sequence of the first end and the second molecule binding sequence of the second end, wherein the intermediate portion comprises:
(I) a first pin forming sequence comprising a barcode,
(II) a second pin forming sequence that is complementary to the first pin forming sequence, wherein the first pin forming sequence is hybridized to the second pin forming sequence, and
(III) a loop linking the first pin forming sequence and the second pin forming sequence;
(b) a first spacer polynucleotide that has a polynucleotide sequence that is complementary to the first pin forming sequence; and
(c) a second spacer polynucleotide that has a polynucleotide sequence that is complementary to the second pin forming sequence,
wherein the first spacer polynucleotide and the second spacer polynucleotide are hybridized to the intermediate portion, and
wherein the nucleic acid scaffold requires a force of 0.1 pN to 10 pN applied to the bead along an axis perpendicular to the bottom surface of the device to stretch the contiguous polynucleotide sequence along the axis to form the nucleic acid scaffold.

2. The nucleic acid scaffold of claim 1, wherein the barcode comprises one or more modified nucleotides.

3. The nucleic acid scaffold of claim 1, wherein the intermediate portion further comprises a first junction sequence and a second junction sequence, wherein the first junction sequence is located between the first molecule binding sequence and the first pin forming sequence, and wherein the second junction sequence is located between the second molecule binding sequence and the second pin forming sequence, and wherein the first junction sequence and the second junction sequence are not complementary to each other.

4. The nucleic acid scaffold of claim 1, wherein the first spacer polynucleotide and the second spacer polynucleotide are linked to each other by a spacer loop sequence, wherein the spacer loop sequence is hybridized to the loop.

5. The nucleic acid scaffold of claim 1, wherein each, the first molecule binding sequence and the second molecule binding sequence, independently comprises a small hairpin nucleic acid having a size in a range of from 5 bases to 100 bases.

6. A screening nucleic acid scaffold comprising:
(a) the nucleic acid scaffold of claim 1;
(b) a first candidate molecule linked to a first glue sequence, wherein the first glue sequence is hybridized to the first molecule binding sequence; and
(c) a second candidate molecule linked to a second glue sequence, wherein the second glue sequence is hybridized to the second molecule binding sequence.

7. A device that comprises:
(a) a chamber disposed within the device, wherein the chamber comprises a bottom surface;
(b) a force application mechanism for stretching the contiguous polynucleotide sequence along the axis to form the nucleic acid scaffold; and
(c) the nucleic acid scaffold of claim 1.

8. A method of determining a binding interaction between a first candidate molecule and a second candidate molecule, the method comprising:
(a) providing a device that comprises:
(i) a chamber disposed within the device, wherein the chamber comprises a bottom surface, and
(ii) a force application mechanism for stretching the contiguous polynucleotide sequence along the axis to form the nucleic acid scaffold;
(b) providing the nucleic acid scaffold of claim 1, wherein the nucleic acid scaffold is positioned along an axis perpendicular to a bottom surface of the chamber of the device;
(c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence;
(d) determining a reference elongation length of the nucleic acid scaffold in response to a force in the absence of the first candidate molecule and the second candidate molecule in real time by:
(iii) applying a force of 0.1 to 50 pN to the bead attached to the nucleic acid scaffold via the force application mechanism along the axis perpendicular to the bottom surface of the device, wherein the nucleic acid scaffold is configured to unfold in response to the force applied, thereby resulting in a change in position of the bead attached to the nucleic acid scaffold along the axis, and
(iv) measuring the change in position of the bead along the axis via a sensor, thereby determining the reference elongation length of the nucleic acid scaffold in the absence of the first candidate molecule and the second candidate molecule;
(e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity with each other;
(f) determining an elongation length of the screening nucleic acid scaffold in response to the same force used in the absence of the first candidate molecule and the second candidate molecule by repeating (d); and
(g) calculating a differential value, wherein the differential value is a difference between the elongation length of the screening nucleic acid scaffold and the reference elongation length at the force applied,
whereby a non-zero differential value indicates presence of the binding interaction between the first candidate molecule and the second candidate molecule at the force applied, and whereby differential value of zero indicates absence of the binding interaction between the first candidate molecule and the second candidate molecule at the force applied.

9. The method of claim 8, further comprising determining binding kinetics of the binding interaction between the first candidate molecule and the second candidate molecule by: (h) removing the force applied by the force application mechanism to the bead after (g), thereby resulting in relaxation of the screening nucleic acid scaffold; and (i) repeating (f)-(h) and calculating a difference between the elongation length of the screening nucleic acid scaffold and the reference elongation length as a function of time.

10. The method of claim 9, further comprising: (i) determining a Kₒₙ for the binding of the second candidate molecule to the first candidate molecule, wherein the Kₒₙ is calculated based on the number of cycles of repeating (f)-(h) that result in the differential value between the elongation length of the screening nucleic acid scaffold and the reference elongation length.

11. The method of claim 9, further comprising: (h) determining a K_{off} for the binding of the second candidate molecule to the first candidate molecule, wherein the K_{off} is calculated based on the length of time that the differential value between the elongation length of the screening nucleic acid scaffold and the reference elongation length is present during each cycle of repeating (f)-(h).

12. A method of determining a binding interaction between a first candidate molecule, a second candidate molecule and a third candidate molecule, the method comprising:
(a) providing a device that comprises:
(i) a chamber disposed within the device, wherein the chamber comprises a bottom surface, and
(ii) a force application mechanism for stretching the contiguous polynucleotide sequence along the axis to form the nucleic acid scaffold;
(b) providing the nucleic acid scaffold of claim 1, wherein the nucleic acid scaffold is positioned along an axis perpendicular to a bottom surface of the chamber of the device;
(c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence;
(d) determining a reference elongation length of the nucleic acid scaffold in the absence of the first candidate molecule and the second candidate molecule by:
(i) applying a force of 0.1 pN to 50 pN to the bead attached to the nucleic acid scaffold via the force application mechanism along the axis perpendicular to the bottom surface of the chamber of the device, wherein the nucleic acid scaffold is configured to unfold in response to the force applied, thereby resulting in a change in position of the bead attached to the nucleic acid scaffold along the axis, and
(ii) measuring the change in position of the bead along the axis via a sensor, thereby determining the reference elongation length of the nucleic acid scaffold in the absence of the first candidate molecule and the second candidate molecule;
(e) contacting the nucleic acid scaffold with the first candidate molecule and the second candidate molecule, and, thereby, forming a screening nucleic acid scaffold, wherein the first candidate molecule and the second candidate molecule are positioned along the screening nucleic acid scaffold such that the first candidate molecule and second candidate molecule are in close proximity with each other;
(f) determining a force required to achieve the same elongation length for the screening nucleic acid scaffold as the reference elongation length by repeating (d), wherein the force required is in a range of from 0.1 pN to 50 pN;
(g) contacting the third candidate molecule to the screening nucleic acid scaffold;
(h) determining an elongation length of the screening nucleic acid scaffold in the presence of the third candidate molecule and in response to the same force applied in (f) by repeating (d); and
(i) calculating a differential value, wherein the differential value is a difference between the elongation length of the screening nucleic acid scaffold in the presence and absence of the third candidate molecule,
whereby a non-zero differential value indicates presence of a binding interaction between the third candidate molecule, and the first candidate molecule and the second candidate molecule at the force applied, and whereby a differential value of zero indicates absence of the binding interaction between:
(A) the third candidate molecule and the first candidate molecule,
(B) the third candidate molecule and the second candidate molecule, or
(C) the third candidate molecule, and the first candidate molecule and the second candidate molecule, at the force applied.

13. The method of claim 12, further comprising determining binding kinetics of the binding interaction between the first candidate molecule, the second candidate molecule and the third candidate molecule by: (j) removing the force applied by the force application mechanism to the bead after (i), thereby resulting in relaxation of a test screening nucleic acid scaffold; and (k) repeating (e)-(j) and calculating a difference between the elongation length of the screening nucleic acid scaffold in the presence and absence of the third candidate molecule as a function of time.

14. The method of claim 12, further comprising: (j) determining a Kₒₙ for the binding of the third candidate molecule to the first and second candidate molecules, wherein the Kₒₙ is calculated based on the number of cycles of repeating (h)-(j) that result in the differential value between the elongation length of the screening nucleic acid scaffold in the presence and absence of the third candidate molecule.

15. The method of claim 12, further comprising: (j) determining a K_{off} for the binding of the third candidate molecule to the first and second candidate molecules, wherein the K_{off} is calculated based on the length of time that the differential value between the elongation length of the screening nucleic acid scaffold in the presence and absence of the third candidate molecule is present during each cycle of repeating (h)-(j).

16. A method of screening binding interactions between a first candidate molecule and a plurality of second candidate molecules, the method comprising:
(a) providing a device that comprises:
(i) a chamber disposed within the device, wherein the chamber comprises a bottom surface,
(ii) a force application mechanism, and
(iii) a plurality of nucleic acid scaffolds positioned along an axis perpendicular to the bottom surface of the chamber of the device, wherein each nucleic acid scaffold among the plurality of nucleic acid scaffolds comprises the nucleic acid scaffold of claim 1, wherein each of the plurality of nucleic acid scaffolds are linked to a bead at one end and a feature of the bottom surface of the chamber of the device at the other end;
(b) determining a reference elongation length of each of the nucleic acid scaffold among the plurality of nucleic acid scaffolds in the absence of the first candidate molecule and the plurality of second candidate molecules by:
(i) applying a force of 0.1 pN to 50 pN to the bead attached to each of the plurality of nucleic acid scaffolds via the force application mechanism along the axis perpendicular to the bottom surface of the chamber of the device, wherein each of the plurality of nucleic acid scaffolds is configured to unfold in response to the force applied, thereby resulting in a change in position of the bead attached to each of the plurality of nucleic acid scaffolds along the axis, and
(ii) measuring the change in position of the bead along the axis via a sensor, thereby determining reference elongation lengths for each of the plurality of nucleic acid scaffolds in the absence of the first candidate molecule and the plurality of second candidate molecules;
(c) contacting the plurality of nucleic acid scaffolds with the first candidate molecule linked to a first glue sequence, thereby anchoring the first candidate molecule to each of the plurality of nucleic acid scaffolds, wherein the first glue sequence is complementary to the first molecule binding sequence;
(d) contacting the plurality of nucleic acid scaffolds with the plurality of second candidate molecules each linked to a second glue sequence, thereby anchoring one of the second candidate molecule among the plurality of second candidate molecules to each of the plurality of nucleic acid scaffolds, wherein the second glue sequence is complementary to the second molecule binding sequence, thereby forming a plurality of screening nucleic acid scaffolds, wherein the first candidate molecule and the second candidate molecule of each of the plurality of screening nucleic acid scaffolds are positioned such that the first candidate molecule and the second candidate molecule are in close proximity with each other;
(e) determining elongation lengths for each of the plurality of screening nucleic acid scaffolds in response to the force of 0.1 pN to 50 pN by repeating (d); and
(f) calculating a difference between the reference elongation length and the elongation length of each screening nucleic acid scaffold among the plurality of screening nucleic acid scaffolds,
whereby a differential between the elongation length of a screening nucleic acid scaffold and the reference elongation length indicates that the first candidate molecule and the second candidate molecule anchored to the screening nucleic acid scaffold have a binding interaction to each other at the force applied, and whereby the absence of the differential indicates absence of binding interaction between the first candidate molecule and the second candidate molecule at the force applied.

17. The method of claim 16, wherein at least two of the plurality of nucleic acid scaffolds comprise barcode sequences that are located at non-identical positions relative to each other.

18. The method of claim 16, wherein at least two of the plurality of nucleic acid scaffolds comprise a barcode sequence that are non-identical relative to each other.

19. The method of claim 17, further comprising determining identity of the contiguous polynucleotide sequence based on the barcode prior to (b).

20. The method of claim 19, wherein the identity of the contiguous polynucleotide sequence is determined by detecting the position of one or more modified nucleotides in the barcode.

21. A method of screening binding interactions between a first candidate molecule, a second candidate molecule and a plurality of third candidate molecules, the method comprising:
(a) providing a device that comprises:
(i) a chamber disposed within the device, wherein the chamber comprises a bottom surface,
(ii) a force application mechanism, and
(iii) a nucleic acid scaffold positioned along an axis perpendicular to the bottom surface of the chamber of the device, wherein the nucleic acid scaffold comprises the nucleic acid scaffold of claim 1, wherein the nucleic acid scaffold is linked to a bead at one end and a feature of the bottom surface of the chamber of the device at the other end;
(b) determining a reference elongation length of the nucleic acid scaffold in the absence of the first candidate molecule, the second candidate molecule, and the plurality of third candidate molecules by:
(iv) applying a force of 0.1 pN to 50 pN to the bead attached to the nucleic acid scaffold via the force application mechanism along the axis perpendicular to the bottom surface of the chamber of the device, wherein the nucleic acid scaffold is configured to unfold in response to the force applied, thereby resulting in a change in position of the bead attached to the nucleic acid scaffold along the axis, and
(v) measuring the change in position of the bead along the axis via a sensor, thereby determining the reference elongation length for the nucleic acid scaffold in the absence of the first candidate molecule, the second candidate molecule, and the plurality of third candidate molecules;
(c) contacting the nucleic acid scaffold with the first candidate molecule linked to a first glue sequence, thereby anchoring the first candidate molecule to the nucleic acid scaffold;
(d) contacting the nucleic acid scaffold with the second candidate molecule linked to a second glue sequence, thereby anchoring the second candidate molecule to the nucleic acid scaffold, thereby forming a screening nucleic acid scaffold, wherein the first candidate molecule and the second candidate molecule of the screening nucleic acid scaffold are positioned such that the first candidate molecule and the second candidate molecule are in close proximity with each other;
(e) determining a force required to achieve the same elongation length for the screening nucleic acid scaffold as the reference elongation length by repeating (d), wherein the force is in a range of from 0.1 pN to 50 pN;
(f) contacting the plurality of third candidate molecules to the screening nucleic acid scaffold;
(g) determining elongation lengths for the screening nucleic acid scaffold in the presence of at least one of the plurality of third candidate molecules in response to the same force applied in (e) by repeating (b); and
(h) calculating a difference between the reference elongation length and the elongation length of screening nucleic acid scaffold,
whereby a non-zero differential value indicates presence of a binding interaction between the plurality of third candidate molecules, and the first candidate molecule and the second candidate molecule at the force applied, and whereby a differential value of zero indicates absence of the binding interaction between:
(A) the plurality of third candidate molecules and the first candidate molecule,
(B) the plurality of third candidate molecules and the second candidate molecule, or
(C) the plurality of third candidate molecules, and the first candidate molecule and the second candidate molecule, at the force applied.

22. A method of determining a binding interaction between a first candidate molecule and a second candidate molecule, the method comprising:
(a) providing a device that comprises:
(i) a chamber disposed within the device, wherein the chamber comprises a bottom surface, and
(ii) a force application mechanism for stretching the contiguous polynucleotide sequence along the axis to form the nucleic acid scaffold;
(b) providing the nucleic acid scaffold of claim 1, wherein the nucleic acid scaffold is positioned along an axis perpendicular to a bottom surface of the chamber of the device;
(c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence;
(d) determining a reference amplitude of Brownian noise of the nucleic acid scaffold in response to a force of less than 0.01 pN in the absence of the first candidate molecule and the second candidate molecule;
(e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact with each other;
(f) determining an amplitude of Brownian noise of the screening nucleic acid scaffold in response to the same force used in the absence of the first candidate molecule and the second candidate molecule by repeating (d); and
(g) identify events of interaction between the first candidate molecule and the second candidate molecule, wherein the amplitude of Brownian noise is reduced compared to the reference amplitude at the same force in the absence of the first candidate molecule and the second candidate molecule.

23. A method of determining a binding interaction between a first candidate molecule, a second candidate molecule and a third candidate molecule, the method comprising:
(a) providing a device that comprises:
(i) a chamber disposed within the device, wherein the chamber comprises a bottom surface, and
(ii) a force application mechanism for stretching the contiguous polynucleotide sequence along the axis to form the nucleic acid scaffold;
(b) providing the nucleic acid scaffold of claim 1, wherein the nucleic acid scaffold is positioned along an axis perpendicular to a bottom surface of the chamber of the device;
(c) providing a first candidate molecule linked to a first glue sequence, and a second candidate molecule linked to a second glue sequence, wherein the first glue sequence is complementary to the first molecule binding sequence, and wherein the second glue sequence is complementary to the second molecule binding sequence;
(d) determining a reference amplitude of Brownian noise of the nucleic acid scaffold in response to a force of less than 0.01 pN in the absence of the first candidate molecule and the second candidate molecule;
(e) contacting the nucleic acid scaffold with the first candidate molecule linked to the first glue sequence, and the second candidate molecule linked to the second glue sequence, and, thereby, attaching the first candidate molecule and the second candidate molecule to the first molecule binding sequence and the second molecule binding sequence, respectively, and forming a screening nucleic acid scaffold, wherein the first candidate molecule and the second candidate molecule are positioned along the screening nucleic acid scaffold such that the first candidate molecule and second candidate molecule are in close proximity or in contact with each other;
(f) contacting a third candidate molecule in solution to the screening nucleic acid scaffold;
(g) determining an amplitude of Brownian noise of the screening nucleic acid scaffold in the presence of the third candidate molecule in response to the same force applied in (d); and
(h) identify events of interaction between the first candidate molecule, the second candidate molecule and the third candidate molecule, wherein the amplitude of Brownian noise is reduced compared to the reference amplitude at the same force in the absence of the first candidate molecule, the second candidate molecule and the third candidate molecule.

24. A kit for determining a binding interaction between a first candidate molecule and a second candidate molecule, the kit comprising:
(a) a nucleic acid that comprises:
(i) a contiguous polynucleotide sequence comprising:
(I) a first end that comprises: (i) a first adapter for attaching to a bead, and (ii) a first molecule binding sequence;
(II) a second end that comprises: (i) a second adapter for attaching the nucleic acid to a bottom surface of a device, and (ii) a second molecule binding sequence;
(III) an intermediate portion between the first molecule binding sequence of the first end and the second molecule binding sequence of the second end, wherein the intermediate portion comprises:
A. a first pin forming sequence comprising a barcode,
B. a second pin forming sequence that is complementary to the first pin forming sequence, wherein the first pin forming sequence is hybridized to the second pin forming sequence, and
C. a loop linking the first pin forming sequence and the second pin forming sequence;
(ii) a first spacer polynucleotide that has a polynucleotide sequence that is complementary to the first pin forming sequence, and
(iii) a second spacer polynucleotide that has a polynucleotide sequence that is complementary to the second pin forming sequence,
wherein the nucleic acid when attached as a nucleic acid scaffold to the bottom surface of the device requires a force of 10 pN to 30 pN to unfold the contiguous polynucleotide sequence along an axis perpendicular to the bottom surface of the device; and
(b) a bead comprising an anchoring molecule configured to bind to the first adapter of the first end of the contiguous polynucleotide sequence of the nucleic acid.

25. The kit of claim 24, wherein each, the first molecule binding sequence and the second molecule binding sequence, independently comprises a small hairpin nucleic acid having a size in a range of from 5 to 100 bases.

26. The kit of claim 24 further comprising:
(a) a first glue sequence comprising a first active group that is configured to be linked with a first candidate molecule, wherein the first glue sequence is complementary to the first molecule binding sequence; and
(b) a second glue sequence comprising a second active group that is configured to be linked with a second candidate molecule, wherein the second glue sequence is complementary to the second molecule binding sequence.

27. The kit of claim 24 further comprising at least one of the first candidate molecule and the second candidate molecule.

28. The kit of claim 24 further comprising a third candidate molecule.

29. The kit of claim 24, wherein the first spacer polynucleotide and the second spacer polynucleotide are linked to each other by a spacer loop sequence, wherein the spacer loop sequence is hybridized to the loop.
